SEQUENCE LISTING (185 sequences)

SEQ ID NO: 1 - protein, 25 aa, synthetic construct
QVQLVESGGALVQPGGSLRLSCSAS

SEQ ID NO: 2 - protein, 8 aa, synthetic construct
GFTFSKYL

SEQ ID NO: 3 - protein, 17 aa, synthetic construct
MSWYRQAPGKERELVAT

SEQ ID NO: 4 - protein, 8 aa, synthetic construct
ITTAGGNT

SEQ ID NO: 5 - protein, 38 aa, synthetic construct
NYADSVKGRFTISRDNAKNTVYLQMNSLKPEDTAVYYC

SEQ ID NO: 6 - protein, 14 aa, synthetic construct
KASGGTIRTVNETY

SEQ ID NO: 7 - protein, 11 aa, synthetic construct
WGQGTQVTVSS

SEQ ID NO: 8 - protein, 121 aa, synthetic construct
QVQLVESGGALVQPGGSLRLSCSASGFTFSKYLMSWYRQAPGKERELVATITTAGGNTNYADSVKGRFTISRDNAKNTVYLQMNSLKPEDTAVYYCKASGGTIRTVNETYWGQGTQVTVSS

SEQ ID NO: 9 - protein, 25 aa, synthetic construct
QVQLVESGGGLVQPGGSLRLSCVDS

SEQ ID NO: 10 - protein, 8 aa, synthetic construct
RSWINVYG

SEQ ID NO: 11 - protein, 17 aa, synthetic construct
ANWYRQAPGKERELVAA

SEQ ID NO: 12 - protein, 7 aa, synthetic construct
LTSGGTT

SEQ ID NO: 13 - protein, 38 aa, synthetic construct
NYADSVKGRFTISRDNAKNTVYLQMRDLKPEDTAVYYC

SEQ ID NO: 14 - protein, 12 aa, synthetic construct
NLERYTGSSVYP

SEQ ID NO: 15 - protein, 11 aa, synthetic construct
WGQGTQVTVSS

SEQ ID NO: 16 - protein, 118 aa, synthetic construct
QVQLVESGGGLVQPGGSLRLSCVDSRSWINVYGANWYRQAPGKERELVAALTSGGTTNYADSVKGRFTISRDNAKNTVYLQMRDLKPEDTAVYYCNLERYTGSSVYPWGQGTQVTVSS

SEQ ID NO: 17 - protein, 25 aa, synthetic construct
QVQLVETGGGLVQPGGSLRLSCAAS

SEQ ID NO: 18 - protein, 8 aa, synthetic construct
GFTLDIYN

SEQ ID NO: 19 - protein, 18 aa, synthetic construct
MGWFRQAPGKEREGVSCI

SEQ ID NO: 20 - protein, 8 aa, synthetic construct
SSRYGTST

SEQ ID NO: 21 - protein, 38 aa, synthetic construct
YYADSVKGRFTISRDNAKNTVYLQMNNLKPEDTAVYYC

SEQ ID NO: 22 - protein, 22 aa, synthetic construct
ATVSWRFTTNQDVCLTPSVSGY

SEQ ID NO: 23 - protein, 11 aa, synthetic construct
WGQGTQVTVSS

SEQ ID NO: 24 - protein, 130 aa, synthetic construct
QVQLVETGGGLVQPGGSLRLSCAASGFTLDIYNMGWFRQAPGKEREGVSCISSRYGTSTYYADSVKGRFTISRDNAKNTVYLQMNNLKPEDTAVYYCATVSWRFTTNQDVCLTPSVSGYWGQGTQVTVSS

SEQ ID NO: 25 - protein, 25 aa, synthetic construct
QVQLVESGGGLVQPGGSLRLSCAAS

SEQ ID NO: 26 - protein, 8 aa, synthetic construct
GFTLDRHA

SEQ ID NO: 27 - protein, 18 aa, synthetic construct
IAWFRQAPGLEREGVVCT

SEQ ID NO: 28 - protein, 7 aa, synthetic construct
SSSGAIT

SEQ ID NO: 29 - protein, 38 aa, synthetic construct
NTADSVKGRFTISRDDAKNTVYLQMNSLKPEDTAIYYC

SEQ ID NO: 30 - protein, 21 aa, synthetic construct
AVRELWTVQPICEPRPRAYAY

SEQ ID NO: 31 - protein, 11 aa, synthetic construct
WGQGTQVTVSS

SEQ ID NO: 32 - protein, 128 aa, synthetic construct
QVQLVESGGGLVQPGGSLRLSCAASGFTLDRHAIAWFRQAPGLEREGVVCTSSSGAITNTADSVKGRFTISRDDAKNTVYLQMNSLKPEDTAIYYCAVRELWTVQPICEPRPRAYAYWGQGTQVTVSS

SEQ ID NO: 33 - protein, 25 aa, synthetic construct
QVQLVETGGGLVQPGGSLRLSCAAS

SEQ ID NO: 34 - protein, 8 aa, synthetic construct
GFVFSINP

SEQ ID NO: 35 - protein, 17 aa, synthetic construct
MNWYRQAPGKERELVAY

SEQ ID NO: 36 - protein, 8 aa, synthetic construct
ISMRGSTT

SEQ ID NO: 37 - protein, 38 aa, synthetic construct
NYADSVKGRFIISRDNAKNTLYLQMNSLKPEDTAVYYC

SEQ ID NO: 38 - protein, 7 aa, synthetic construct
NAPSRSV

SEQ ID NO: 39 - protein, 11 aa, synthetic construct
WGQGTQVTVSS

SEQ ID NO: 40 - protein, 114 aa, synthetic construct
QVQLVETGGGLVQPGGSLRLSCAASGFVFSINPMNWYRQAPGKERELVAYISMRGSTTNYADSVKGRFIISRDNAKNTLYLQMNSLKPEDTAVYYCNAPSRSVWGQGTQVTVSS

SEQ ID NO: 41 - protein, 25 aa, synthetic construct
QVQLVETGGGLVQPGGSLRLSCTAS

SEQ ID NO: 42 - protein, 8 aa, synthetic construct
GFIFSANQ

SEQ ID NO: 43 - protein, 17 aa, synthetic construct
MNWVRQAPGKGLEWLSG

SEQ ID NO: 44 - protein, 8 aa, synthetic construct
ISTRGDTT

SEQ ID NO: 45 - protein, 38 aa, synthetic construct
SYADSVKGRFTISRDNAKNTLYLQMNSLQPDDTAVYFC

SEQ ID NO: 46 - protein, 16 aa, synthetic construct
ARVCIRGPEPKLRCDD

SEQ ID NO: 47 - protein, 11 aa, synthetic construct
WGQGTQVTVSS

SEQ ID NO: 48 - protein, 123 aa, synthetic construct
QVQLVETGGGLVQPGGSLRLSCTASGFIFSANQMNWVRQAPGKGLEWLSGISTRGDTTSYADSVKGRFTISRDNAKNTLYLQMNSLQPDDTAVYFCARVCIRGPEPKLRCDDWGQGTQVTVSS

SEQ ID NO: 49 - protein, 25 aa, synthetic construct
QVQLVESGGGLVQAGGSLRLSCAAS

SEQ ID NO: 50 - protein, 8 aa, synthetic construct
EHIFTSSA

SEQ ID NO: 51 - protein, 17 aa, synthetic construct
MGWFRQVSGKEREFVAA

SEQ ID NO: 52 - protein, 8 aa, synthetic construct
ISWSGISQ

SEQ ID NO: 53 - protein, 38 aa, synthetic construct
YYADSVKGRFTISRDNAKNTVYLQMNSLKPEDTAVYYC

SEQ ID NO: 54 - protein, 20 aa, synthetic construct
AADGGYKGTYDYLSPSWYNY

SEQ ID NO: 55 - protein, 11 aa, synthetic construct
WGQGTQVTVSS

SEQ ID NO: 56 - protein, 127 aa, synthetic construct
QVQLVESGGGLVQAGGSLRLSCAASEHIFTSSAMGWFRQVSGKEREFVAAISWSGISQYYADSVKGRFTISRDNAKNTVYLQMNSLKPEDTAVYYCAADGGYKGTYDYLSPSWYNYWGQGTQVTVSS

SEQ ID NO: 57 - protein, 25 aa, synthetic construct
QLQLVETGGGLVQAGGSLRLSCAAS

SEQ ID NO: 58 - protein, 8 aa, synthetic construct
GRTFSSYA

SEQ ID NO: 59 - protein, 17 aa, synthetic construct
MGWFRQAPGKEREFVAV

SEQ ID NO: 60 - protein, 8 aa, synthetic construct
ISQSGGGT

SEQ ID NO: 61 - protein, 38 aa, synthetic construct; identical to SEQ ID NO: 53

SEQ ID NO: 62 - protein, 14 aa, synthetic construct
AADPSGSTARVWDY

SEQ ID NO: 63 - protein, 11 aa, synthetic construct
WGQGTQVTVSS

SEQ ID NO: 64 - protein, 121 aa, synthetic construct
QLQLVETGGGLVQAGGSLRLSCAASGRTFSSYAMGWFRQAPGKEREFVAVISQSGGGTYYADSVKGRFTISRDNAKNTVYLQMNSLKPEDTAVYYCAADPSGSTARVWDYWGQGTQVTVSS

SEQ ID NO: 65 - protein, 25 aa, synthetic construct
QVQLVETGGGLVQAGGSLTLSCVVS

SEQ ID NO: 66 - protein, 8 aa, synthetic construct
GRTFGSYA

SEQ ID NO: 67 - protein, 17 aa, synthetic construct
MGWFRQAPGKEREFVGT

SEQ ID NO: 68 - protein, 8 aa, synthetic construct
ISWSGGRT

SEQ ID NO: 69 - protein, 38 aa, synthetic construct
YYADSVKGRFTISRDNAKNTVYLQMNSLEPEDTAIYYC

SEQ ID NO: 70 - protein, 20 aa, synthetic construct
AIPTYGRKWYPTAQAQDFDS

SEQ ID NO: 71 - protein, 11 aa, synthetic construct
WGQGTQVTVSS

SEQ ID NO: 72 - protein, 127 aa, synthetic construct
QVQLVETGGGLVQAGGSLTLSCVVSGRTFGSYAMGWFRQAPGKEREFVGTISWSGGRTYYADSVKGRFTISRDNAKNTVYLQMNSLEPEDTAIYYCAIPTYGRKWYPTAQAQDFDSWGQGTQVTVSS

SEQ ID NO: 73 - protein, 25 aa, synthetic construct
QVQLVETGGGLVQAGDSLRLSCAAS

SEQ ID NO: 74 - protein, 8 aa, synthetic construct
GRTFSRYA

SEQ ID NO: 75 - protein, 17 aa, synthetic construct
MGWFRQAPGKEREFVAV

SEQ ID NO: 76 - protein, 8 aa, synthetic construct
ISQSGGSS

SEQ ID NO: 77 - protein, 38 aa, synthetic construct
YYATSVKGRFFISRDNAKNRLYLQMNSLKPEDTAVYYC

SEQ ID NO: 78 - protein, 14 aa, synthetic construct
AADPSGSTVRPWDY

SEQ ID NO: 79 - protein, 11 aa, synthetic construct
WGQGTQVTVSS

SEQ ID NO: 80 - protein, 121 aa, synthetic construct
QVQLVETGGGLVQAGDSLRLSCAASGRTFSRYAMGWFRQAPGKEREFVAVISQSGGSSYYATSVKGRFFISRDNAKNRLYLQMNSLKPEDTAVYYCAADPSGSTVRPWDYWGQGTQVTVSS

SEQ ID NO: 81 - protein, 25 aa, synthetic construct
QVQLVETGGGLVQAGGSLRLSCAAS

SEQ ID NO: 82 - protein, 8 aa, synthetic construct
EGTLSNYA

SEQ ID NO: 83 - protein, 16 aa, synthetic construct
MGWFRQAPGKEREFVA

SEQ ID NO: 84 - protein, 8 aa, synthetic construct
ISWSGGST

SEQ ID NO: 85 - protein, 38 aa, synthetic construct
YYVDSVKGRFTISRDNAKTTVYLRMNSLKPEDTAVYYC

SEQ ID NO: 86 - protein, 20 aa, synthetic construct
AADGGYKGDYRYTESSWYFY

SEQ ID NO: 87 - protein, 11 aa, synthetic construct
WGQGTQVTVSS

SEQ ID NO: 88 - protein, 126 aa, synthetic construct
QVQLVETGGGLVQAGGSLRLSCAASEGTLSNYAMGWFRQAPGKEREFVAISWSGGSTYYVDSVKGRFTISRDNAKTTVYLRMNSLKPEDTAVYYCAADGGYKGDYRYTESSWYFYWGQGTQVTVSS

SEQ ID NO: 89 - protein, 25 aa, synthetic construct
QVQLVETGGGLVQAGGSLRLSCAAS

SEQ ID NO: 90 - protein, 8 aa, synthetic construct
ERTFSSSA

SEQ ID NO: 91 - protein, 17 aa, synthetic construct
MGWFRQVPGKEREFVAA

SEQ ID NO: 92 - protein, 8 aa, synthetic construct
VSWSGSSQ

SEQ ID NO: 93 - protein, 38 aa, synthetic construct
YYADSVKGRFTISRDNAKNMVYLQMNSLKPEDTAVYYC

SEQ ID NO: 94 - protein, 20 aa, synthetic construct
AADGGYKGYYDYLSPSWYNY

SEQ ID NO: 95 - protein, 11 aa, synthetic construct
WGQGTQVTVSS

SEQ ID NO: 96 - protein, 127 aa, synthetic construct
QVQLVETGGGLVQAGGSLRLSCAASERTFSSSAMGWFRQVPGKEREFVAAVSWSGSSQYYADSVKGRFTISRDNAKNMVYLQMNSLKPEDTAVYYCAADGGYKGYYDYLSPSWYNYWGQGTQVTVSS

SEQ ID NO: 97 - protein, 25 aa, synthetic construct
QVQLVESGGGLVQAGGSLRLACVAS

SEQ ID NO: 98 - protein, 8 aa, synthetic construct
GRTFSSDA

SEQ ID NO: 99 - protein, 17 aa, synthetic construct
VSWFRQVPGKEREFVAA

SEQ ID NO: 100 - protein, 5 aa, synthetic construct
IGLGT

SEQ ID NO: 101 - protein, 38 aa, synthetic construct
FYENSVKGRFTISRDNAKNTAYLQMNSLKPEDTAVYYC

SEQ ID NO: 102 - protein, 17 aa, synthetic construct
ATRRSIVVRNTEDAYDY

SEQ ID NO: 103 - protein, 11 aa, synthetic construct
WGQGTQVTVSS

SEQ ID NO: 104 - protein, 121 aa, synthetic construct
QVQLVESGGGLVQAGGSLRLACVASGRTFSSDAVSWFRQVPGKEREFVAAIGLGTFYENSVKGRFTISRDNAKNTAYLQMNSLKPEDTAVYYCATRRSIVVRNTEDAYDYWGQGTQVTVSS

SEQ ID NO: 105 - protein, 25 aa, synthetic construct
QVQLVESGGGLVQAGGSLRLSCAAS

SEQ ID NO: 106 - protein, 8 aa, synthetic construct
GRTFSSYA

SEQ ID NO: 107 - protein, 17 aa, synthetic construct
MGWFRQAPGKEREFVAV

SEQ ID NO: 108 - protein, 8 aa, synthetic construct
ISQSGGSA

SEQ ID NO: 109 - protein, 38 aa, synthetic construct; identical to SEQ ID NO: 53

SEQ ID NO: 110 - protein, 14 aa, synthetic construct
AADPSGSSVKVWDY

SEQ ID NO: 111 - protein, 11 aa, synthetic construct
WGQGTQVTVSS

SEQ ID NO: 112 - protein, 121 aa, synthetic construct
QVQLVESGGGLVQAGGSLRLSCAASGRTFSSYAMGWFRQAPGKEREFVAVISQSGGSAYYADSVKGRFTISRDNAKNTVYLQMNSLKPEDTAVYYCAADPSGSSVKVWDYWGQGTQVTVSS

SEQ ID NO: 113 - protein, 25 aa, synthetic construct
QLQLVETGGGLVQTGGSLRLSCAAS

SEQ ID NO: 114 - protein, 8 aa, synthetic construct
GRTFSSYA

SEQ ID NO: 115 - protein, 17 aa, synthetic construct
MGWFRQAPGKEREFVAV

SEQ ID NO: 116 - protein, 8 aa, synthetic construct
ISQSGGST

SEQ ID NO: 117 - protein, 38 aa, synthetic construct; identical to SEQ ID NO: 53

SEQ ID NO: 118 - protein, 14 aa, synthetic construct
ADPGGSVQADRWDY

SEQ ID NO: 119 - protein, 11 aa, synthetic construct
WGQGTQVTVSS

SEQ ID NO: 120 - protein, 121 aa, synthetic construct
QLQLVETGGGLVQTGGSLRLSCAASGRTFSSYAMGWFRQAPGKEREFVAVISQSGGSTYYADSVKGRFTISRDNAKNTVYLQMNSLKPEDTAVYYCADPGGSVQADRWDYWGQGTQVTVSS

SEQ ID NO: 121 - protein, 25 aa, synthetic construct
QLQLVESGGGLVQAGGSLRLSCAAS

SEQ ID NO: 122 - protein, 8 aa, synthetic construct
GRTFGSYA

SEQ ID NO: 123 - protein, 17 aa, synthetic construct
MAWFRQAPGKEREFVAV

SEQ ID NO: 124 - protein, 8 aa, synthetic construct
ISSSGGGT

SEQ ID NO: 125 - protein, 38 aa, synthetic construct
YYADSVKGRFTISRDNAKRTVYLQMNSLKPEDTAVYYC

SEQ ID NO: 126 - protein, 16 aa, synthetic construct
AADPKGSIRAWTLVDY

SEQ ID NO: 127 - protein, 11 aa, synthetic construct
WGKGTLVTVSS

SEQ ID NO: 128 - protein, 123 aa, synthetic construct
QLQLVESGGGLVQAGGSLRLSCAASGRTFGSYAMAWFRQAPGKEREFVAVISSSGGGTYYADSVKGRFTISRDNAKRTVYLQMNSLKPEDTAVYYCAADPKGSIRAWTLVDYWGKGTLVTVSS

SEQ ID NO: 129 - protein, 25 aa, synthetic construct
QLQLVETGGGLVQAGGSLRLTCAAS

SEQ ID NO: 130 - protein, 10 aa, synthetic construct
GHTFTFSDYA

SEQ ID NO: 131 - protein, 17 aa, synthetic construct
MGWFRQAPGKEREFVAV

SEQ ID NO: 132 - protein, 8 aa, synthetic construct
IRRSGSST

SEQ ID NO: 133 - protein, 38 aa, synthetic construct; identical to SEQ ID NO: 53

SEQ ID NO: 134 - protein, 17 aa, synthetic construct
AADPHWRISGSGFQLEV

SEQ ID NO: 135 - protein, 11 aa, synthetic construct
WGQGTLVTVSS

SEQ ID NO: 136 - protein, 126 aa, synthetic construct
QLQLVETGGGLVQAGGSLRLTCAASGHTFTFSDYAMGWFRQAPGKEREFVAVIRRSGSSTYYADSVKGRFTISRDNAKNTVYLQMNSLKPEDTAVYYCAADPHWRISGSGFQLEVWGQGTLVTVSS

SEQ ID NO: 137 - protein, 25 aa, synthetic construct
QVQLVETGGGLVQAGGSLRLSCAAS

SEQ ID NO: 138 - protein, 8 aa, synthetic construct
GRTFSTYP

SEQ ID NO: 139 - protein, 17 aa, synthetic construct
MGWFRQAPGKEREFVAV

SEQ ID NO: 140 - protein, 8 aa, synthetic construct
IRWSGSST

SEQ ID NO: 141 - protein, 38 aa, synthetic construct
YYADSVKGRFTISRDNAKNTVYLQMSRLKPEDTAVYYC

SEQ ID NO: 142 - protein, 15 aa, synthetic construct
AADQTWGSRWSLYEY

SEQ ID NO: 143 - protein, 11 aa, synthetic construct
WGQGTQVTVSS

SEQ ID NO: 144 - protein, 122 aa, synthetic construct
QVQLVETGGGLVQAGGSLRLSCAASGRTFSTYPMGWFRQAPGKEREFVAVIRWSGSSTYYADSVKGRFTISRDNAKNTVYLQMSRLKPEDTAVYYCAADQTWGSRWSLYEYWGQGTQVTVSS

SEQ ID NO: 145 - protein, 25 aa, synthetic construct
QLQLVETGGGLVQAGGSLRLSCTDS

SEQ ID NO: 146 - protein, 8 aa, synthetic construct
GRTLSNYA

SEQ ID NO: 147 - protein, 17 aa, synthetic construct
MGWFRQAPGKEREFVAA

SEQ ID NO: 148 - protein, 8 aa, synthetic construct
ISWSGSRT

SEQ ID NO: 149 - protein, 38 aa, synthetic construct
YYADSVKGRFTISRDNANNRVYLQMNSLKPEDTAVYYC

SEQ ID NO: 150 - protein, 20 aa, synthetic construct
AADGGYSGTYYYTESSWYNY

SEQ ID NO: 151 - protein, 11 aa, synthetic construct
WGQGTQVTVSS

SEQ ID NO: 152 - protein, 127 aa, synthetic construct
QLQLVETGGGLVQAGGSLRLSCTDSGRTLSNYAMGWFRQAPGKEREFVAAISWSGSRTYYADSVKGRFTISRDNANNRVYLQMNSLKPEDTAVYYCAADGGYSGTYYYTESSWYNYWGQGTQVTVSS

SEQ ID NO: 153 - protein, 25 aa, synthetic construct
QVQLVETGGGLVQAGGSLRLSCAAS

SEQ ID NO: 154 - protein, 8 aa, synthetic construct
GRTFSTYA

SEQ ID NO: 155 - protein, 17 aa, synthetic construct
MGWFRQRPGKEREFVAV

SEQ ID NO: 156 - protein, 8 aa, synthetic construct
ISGSGGST

SEQ ID NO: 157 - protein, 38 aa, synthetic construct
YYAGSVKGRFTISRDNAKNTVYLQMNSLKPDDTAVYYC

SEQ ID NO: 158 - protein, 18 aa, synthetic construct
AADESGKGSVRTLSYAGS

SEQ ID NO: 159 - protein, 11 aa, synthetic construct
WGQGTQVTVSS

SEQ ID NO: 160 - protein, 125 aa, synthetic construct
QVQLVETGGGLVQAGGSLRLSCAASGRTFSTYAMGWFRQRPGKEREFVAVISGSGGSTYYAGSVKGRFTISRDNAKNTVYLQMNSLKPDDTAVYYCAADESGKGSVRTLSYAGSWGQGTQVTVSS

SEQ ID NO: 161 - protein, 25 aa, synthetic construct
QLQLVESGGGLVQAGGSLRLSCAAS

SEQ ID NO: 162 - protein, 8 aa, synthetic construct
GRTFSSYA

SEQ ID NO: 163 - protein, 17 aa, synthetic construct
MGWFRQAPGKEREFVAA

SEQ ID NO: 164 - protein, 8 aa, synthetic construct
ISGSGDST

SEQ ID NO: 165 - protein, 38 aa, synthetic construct
YYTNSVKGRFTISRDNTKNTVYLQMNGLKPDDTAVYYC

SEQ ID NO: 166 - protein, 16 aa, synthetic construct
AADPTGNWGMRSPPDY

SEQ ID NO: 167 - protein, 11 aa, synthetic construct
WGQGTQVTVSS

SEQ ID NO: 168 - protein, 123 aa, synthetic construct
QLQLVESGGGLVQAGGSLRLSCAASGRTFSSYAMGWFRQAPGKEREFVAAISGSGDSTYYTNSVKGRFTISRDNTKNTVYLQMNGLKPDDTAVYYCAADPTGNWGMRSPPDYWGQGTQVTVSS

SEQ ID NO: 169 - protein, 25 aa, synthetic construct
QVQLVETGGGLVQTGGSLRLSCAAS

SEQ ID NO: 170 - protein, 8 aa, synthetic construct
GRTFSSYA

SEQ ID NO: 171 - protein, 17 aa, synthetic construct
MGWFRQAPGKEREFVAG

SEQ ID NO: 172 - protein, 8 aa, synthetic construct
ISQNGGST

SEQ ID NO: 173 - protein, 38 aa, synthetic construct
YYTNSVKGRFTISRDNAKNMVYLQMNSLKPEDTAVYHC

SEQ ID NO: 174 - protein, 14 aa, synthetic construct
AADAGGSMRYEWDY

SEQ ID NO: 175 - protein, 11 aa, synthetic construct
WGQGIQVTVSS

SEQ ID NO: 176 - protein, 121 aa, synthetic construct
QVQLVETGGGLVQTGGSLRLSCAASGRTFSSYAMGWFRQAPGKEREFVAGISQNGGSTYYTNSVKGRFTISRDNAKNMVYLQMNSLKPEDTAVYHCAADAGGSMRYEWDYWGQGIQVTVSS

SEQ ID NO: 177 - protein, 25 aa, synthetic construct
QVQLVETGGGLVQAGGSLRLSCAAS

SEQ ID NO: 178 - protein, 8 aa, synthetic construct
GRSFSSYA

SEQ ID NO: 179 - protein, 17 aa, synthetic construct
MGWFRQAPGKEREFVAA

SEQ ID NO: 180 - protein, 8 aa, synthetic construct
ISWSGGSS

SEQ ID NO: 181 - protein, 38 aa, synthetic construct
YYTHSVKGRFTISRDNAKNTVYLQMNSLKPEDTAVYYC

SEQ ID NO: 182 - protein, 18 aa, synthetic construct
AADESGQGSVLALSNAGS

SEQ ID NO: 183 - protein, 11 aa, synthetic construct
WGQGTQVTVSS

SEQ ID NO: 184 - protein, 125 aa, synthetic construct
QVQLVETGGGLVQAGGSLRLSCAASGRSFSSYAMGWFRQAPGKEREFVAAISWSGGSSYYTHSVKGRFTISRDNAKNTVYLQMNSLKPEDTAVYYCAADESGQGSVLALSNAGSWGQGTQVTVSS

SEQ ID NO: 185 - protein, 484 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

(19) United States
(12) Patent Application Publication
(10) Pub. No.: US 2026/0209323 A1
(43) Pub. Date: Jul. 23, 2026

(54) Single-domain antibodies directed against gasdermin D and their use

(71) Applicants: Whitehead Institute for Biomedical Re-search, Cambridge, MA (US); Rheinische Friedrich-Wilhelms-Universität Bonn, Germany (DE); Universitätsklinikum Bonn, Germany (DE)

(72) Inventors: Florian Ingo Schmidt, Germany (DE); Lisa Dominica Jacoba Schiffelers, Netherlands (NL); Sabine Normann, Germany (DE); Matthias Geyer, Germany (DE); Anja Kopp, Germany (DE); Hidde Ploegh, Boston, MA (US)

(21) Appl. No.: 19/279,486

(22) PCT Filed: Feb. 14, 2024
(86) PCT No.: PCT/EP2024/053737

§ 371 Date: Jul. 24, 2025

Related U.S. Application Data
(60) Provisional application No. 63/485,056, filed on Feb. 15, 2023.

(51) Int. Cl. C07K 16/18; G01N 33/68

(52) U.S. Cl. CPC C07K 16/18; G01N 33/68; C07K 2317/14; C07K 2317/565; C07K 2317/567; C07K 2317/569; C07K 2317/76; G01N 2333/4703

(57) ABSTRACT

The present invention is concerned with single-domain antibodies directed against gasdermin D (GSDMD). The single-domain antibodies can be used in medical applications, preferably for preventing and/or treating an inflammatory disease or condition in a subject, and/or for determining the presence or absence of GSDMD oligomers in a sample obtained from a subject.

This invention was made with government support under DP1-GM106409 awarded by NIH. The government has certain rights in the invention.

### TECHNICAL FIELD

The present invention is related to the field of biomedical science and provides single-domain antibodies directed against gasdermin D (GSDMD). The single-domain antibodies can be used in medical applications, preferably for preventing and/or treating an inflammatory disease or condition in a subject, and/or for determining the presence or absence of GSDMD oligomers in a sample obtained from a subject.

### BACKGROUND OF THE INVENTION

Pyroptosis is an inflammatory cell death usually caused by microbial infection, accompanied by activation of inflammasomes and maturation of pro-inflammatory cytokines interleukin-1β (IL-1β) and interleukin-18 (IL-18). Pyroptosis exerts tumor suppression function and evokes anti-tumor immune responses. Therapeutic regimens, including chemotherapy, radiotherapy, targeted therapy and immune therapy, induce pyroptosis in cancer, which potentiate local and systemic anti-tumor immunity. On the other hand, pyroptosis of normal cells attributes to side effects of anti-cancer therapies. Furthermore, dysregulation of the inflammatory response is a key driver in many debilitating diseases linked to acute and chronic inflammation such as sepsis, atherosclerosis, inflammatory bowel disease, non-alcoholic steatohepatitis, lung cancer, familial Mediterranean fever and autoinflammatory diseases such as cryopyrin-associated periodic syndromes. Inhibitors that specifically target pyroptotic cell death may therefore be therapeutically useful in the clinic for the treatment of these diseases.

Gasdermin family proteins are the enforcers of pyroptosis, mediating the final common step of all inflammasome pathways. Gasdermin D (GSDMD) is a pore forming protein and perforates the plasma membrane to execute pyroptosis. GSDMD pore formation is critical for the release of pro-inflammatory cytokines interleukin-1β (IL-1β) and interleukin-18 (IL-18) downstream of canonical inflammasome activation. GSDMD pore formation causes cell death by pyroptosis after canonical and non-canonical inflammasome activation. Aberrant activation of IL-1β and IL-18 signaling and pyroptosis is the molecular basis of most pathologies involving systemic inflammation, including mono- and polygenetic autoinflammatory diseases, autoinflammatory disease caused by abiotic factors and infection (e.g. SARS-CoV-2), as well as certain types of cancer driven by inflammation. GSDMD is thus considered the key effector protein of the inflammasome pathway.

Inflammasomes are cytoplasmic multiprotein complexes comprising a sensor protein, inflammatory caspases, and in some but not all cases an adapter protein connecting the two. They can be activated by a repertoire of endogenous and exogenous stimuli, leading to enzymatic activation of canonical caspase-1, noncanonical caspase-11 (or the equivalent caspase-4 and caspase-5 in humans) or caspase-8, resulting in secretion of IL-1β and IL-18, as well as pyroptotic (caspase-1, -11, -4, -5) or apoptotic (caspase-8) cell death.

The ensuing activation of caspase-1 is not only responsible for the maturation of pro-inflammatory cytokines IL-1β and IL-18, but also for the cleavage of GSDMD in its interdomain linker. This releases the N-terminus (GSDMD^{NT}) from the control of the autoinhibitory C-terminus, allowing GSDMD^{NT} to assemble pores in the plasma membrane. As a result, mature cytokines IL-1β and IL-18 and potentially other pro-inflammatory danger-associated molecular patterns (DAMPs) are released and the plasma membrane becomes permeable to DNA intercalating dyes such as propidium iodide or DRAQ7. Eventually, the entire cell ruptures and releases larger cytosolic components, including tetrameric lactate dehydrogenase (LDH).

GSDMD pores reconstituted in vitro are composed of 31-34 monomers, forming a pore with an estimated inner diameter of 22 nm. After cleavage, GSDMD^{NT} undergoes dramatic conformational changes involving the transition of the short beta sheets and helices in the extension domains into two beta hairpins with extended beta sheets that constitute the membrane spanning pore. It is unclear if loss of GSDMD^{CT} is sufficient to trigger the conformational changes, or whether this only occurs in concert with oligomerization. Apart from assays reporting the permeability of the plasma membrane to different dyes or cell death, pore formation of endogenous GSDMD had not been studied in molecular detail in living cells, largely due to the lack of suitable tools. Pyroptotic cells are very delicate and are not compatible with staining methods involving fixation and multiple washing steps. Moreover, upon inflammasome activation, fluorescent derivatives GSDMD^{NT} were barely observed in the plasma membrane, but mostly in intracellular compartments or structures.

As oligomerization of GSDMD has been linked to pyroptosis, there is a need for a diagnostic tool to identify if oligomerization of GSDMD is occurring in a cellular system, and there is also a need for a therapeutic tool to prevent or stop the processes linked or caused by oligomerization of GSDMD.

Both of these goals were achieved by the present invention.

To provide more insights into oligomerization of GSDMD leading to pore formation and ultimately pyroptosis in a living cell model, single-domain antibodies, also termed nanobodies, against the human GSDMD protein were generated. Nanobodies are single domain antibodies derived from the variable domain of heavy chain-only antibodies (VHH) present in camelids. Due to their small size, specificity, and functionality in the cytosol, they present themselves as useful tools to study target proteins in a cellular system. Several antagonistic GSDMD nanobodies were generated that inhibit pyroptosis and IL-1β release by blocking oligomerization of GSDMD^{NT}. As nanobody-bound GSDMD^{NT} still partitions into the plasma membrane, it was concluded that monomeric GSDMD^{NT} exhibits a suitable conformation to insert into the plasma membrane and only oligomerizes after insertion. It was surprisingly established in the present invention that there exists an unexpected layer of negative caspase-1 regulation by functional GSDMD pores and it was found that the inhibitory nanobodies show great potential in preventing inflammatory cell death in primary human macrophages when administered to the extracellular environment. This is of particular interest since GSDMD is linked to an ever-growing list of (auto)inflammatory, metabolic, and neurodegenerative diseases and cancer and is thus an eminent drug target.

### SUMMARY OF THE INVENTION

In the following, the present invention is described in detail. The features of the present invention are described in individual paragraphs. This, however, does not mean that a feature described in a paragraph stands isolated from a feature or features described in other paragraphs. Rather, a feature described in a paragraph can be combined with a feature or features described in other paragraphs.

The present invention is concerned with single-domain antibodies directed against GSDMD.

The single-domain antibodies of the present invention are characterized by comprising an amino acid sequence comprising framework region 1 (FR1), complementarity-determining region 1 (CDR1), FR2, CDR2, FR3, CDR3, and FR4. FR1 is located at the N-terminal side of the amino acid chain, and FR4 is located at the C-terminal side of the amino acid chain.

### GSDMD1 and Variants

In one embodiment, a single-domain antibody of the present invention is characterized by comprising CDR1: GFTFSKYL (SEQ ID NO:2), CDR2: ITTAGGNT (SEQ ID NO:4), and CDR3: KASGGTIRTVNETY (SEQ ID NO: 6). Additionally, the single-domain antibody can comprise FR1: QVQLVESGGALVQPGGSLRLSCSAS (SEQ ID NO:1), FR2: MSWYRQAPGKERELVAT (SEQ ID NO:3), FR3: NYADSVKGRFTISRDNAKNTVYLQMNSLKPEDTAVYYC (SEQ ID NO:5), and FR4: WGQGTQVTVSS (SEQ ID NO: 7).

The single-domain antibody of the present invention can also be characterized by comprising the amino acid sequence as defined in SEQ ID NO: 8, which consists of SEQ NOS 1 to 7. The single-domain antibody of the present invention consisting of the amino acid sequence as defined by SEQ ID NO:8 is called VHH AK-A04, VHH_{GSDMD-1}, GSDMD1 or VHH-1, alternatively.

The single-domain antibody of the present invention can also be characterized by being a variant of a single-domain antibody of the present invention characterized by comprising the amino acid sequence as defined in SEQ ID NO: 8. The variant is characterized by comprising an amino acid sequence, wherein the amino acid sequence is at least 80%, 90%, 95%, or 99% identical to the amino acid sequence as defined in SEQ ID NO: 8. In a preferred embodiment, the variant comprises the CDR regions as defined in SEQ ID NOS 2, 4, and 6, and the variation occurs in the frame work regions.

### GSDMD2 and Variants

In one embodiment, a single-domain antibody of the present invention is characterized by comprising CDR1: RSWINVYG (SEQ ID NO:10), CDR2: LTSGGTT (SEQ ID NO:12), and CDR3: NLERYTGSSVYP (SEQ ID NO: 14). Additionally, the single-domain antibody can comprise FR1: QVQLVESGGGLVQPGGSLRLSCVDS (SEQ ID NO:9), FR2: ANWYRQAPGKERELVAA (SEQ ID NO:11), FR3: NYADSVKGRFTISRDNAKNTVYLQMRDLKPEDTAVYYC (SEQ ID NO:13), and FR4: WGQGTQVTVSS (SEQ ID NO: 15).

The single-domain antibody of the present invention can also be characterized by comprising the amino acid sequence as defined in SEQ ID NO: 16, which consists of SEQ NOS 9 to 15. The single-domain antibody of the present invention consisting of the amino acid sequence as defined by SEQ ID NO:16 is called VHH AK-C03, VHH_{GSDMD-2}, GSDMD2 or VHH-2.

The single-domain antibody of the present invention can also be characterized by being a variant of a single-domain antibody of the present invention characterized by comprising the amino acid sequence as defined in SEQ ID NO: 16. The variant is characterized by comprising an amino acid sequence, wherein the amino acid sequence is at least 80%, 90%, 95%, or 99% identical to the amino acid sequence as defined in SEQ ID NO: 16. In a preferred embodiment, the variant comprises the CDR regions as defined in SEQ ID NOS 10, 12, and 14, and the variation occurs in the frame work regions.

### GSDMD3 and Variants

In one embodiment, a single-domain antibody of the present invention is characterized by comprising CDR1 as defined by SEQ ID NO:18, CDR2: as defined by SEQ ID NO:20, and CDR3 as defined by SEQ ID NO: 22. Additionally, the single-domain antibody can comprise FR1 as defined by SEQ ID NO:17, FR2: as defined by SEQ ID NO:19, FR3 as defined by SEQ ID NO:21, and FR4 as defined by SEQ ID NO: 23.

The single-domain antibody of the present invention can also be characterized by comprising the amino acid sequence as defined in SEQ ID NO: 24, which consists of SEQ NOS: 17 to 23. The single-domain antibody of the present invention consisting of the amino acid sequence as defined by SEQ ID NO:24 is called VHH AK-H12, GSDMD3 or VHH-3.

The single-domain antibody of the present invention can also be characterized by being a variant of a single-domain antibody of the present invention characterized by comprising the amino acid sequence as defined in SEQ ID NO: 24. The variant is characterized by comprising an amino acid sequence, wherein the amino acid sequence is at least 80%, 90%, 95%, or 99% identical to the amino acid sequence as defined in SEQ ID NO: 24. In a preferred embodiment, the variant comprises the CDR regions as defined in SEQ ID NOS 18, 20, and 22, and the variation occurs in the frame work regions.

### GSDMD4 and Variants

In one embodiment, a single-domain antibody of the present invention is characterized by comprising CDR1 as defined by SEQ ID NO:26, CDR2 as defined by SEQ ID NO:28, and CDR3 as defined by SEQ ID NO: 30. Additionally, the single-domain antibody can comprise FR1 as defined by SEQ ID NO:25, FR2: as defined by SEQ ID NO:27, FR3 as defined by SEQ ID NO:29, and FR4 as defined by SEQ ID NO: 31.

The single-domain antibody of the present invention can also be characterized by comprising the amino acid sequence as defined in SEQ ID NO: 32, which consists of SEQ NOS: 25 to 31. The single-domain antibody of the present invention consisting of the amino acid sequence as defined by SEQ ID NO:32 is called VHH AK-C10, GSDMD4 or VHH-4.

The single-domain antibody of the present invention can also be characterized by being a variant of a single-domain antibody of the present invention characterized by comprising the amino acid sequence as defined in SEQ ID NO: 32. The variant is characterized by comprising an amino acid sequence, wherein the amino acid sequence is at least 80%, 90%, 95%, or 99% identical to the amino acid sequence as defined in SEQ ID NO: 32. In a preferred embodiment, the variant comprises the CDR regions as defined in SEQ ID NOS 26, 28, and 30, and the variation occurs in the frame work regions.

### GSDMD5 and Variants

In one embodiment, a single-domain antibody of the present invention is characterized by comprising CDR1 as defined by SEQ ID NO:34, CDR2 as defined by SEQ ID NO:36, and CDR3 as defined by SEQ ID NO: 38. Additionally, the single-domain antibody can comprise FR1 as defined by SEQ ID NO:33, FR2: as defined by SEQ ID NO:35, FR3 as defined by SEQ ID NO:37, and FR4 as defined by SEQ ID NO: 39.

The single-domain antibody of the present invention can also be characterized by comprising the amino acid sequence as defined in SEQ ID NO: 40, which consists of SEQ NOS: 33 to 39. The single-domain antibody of the present invention consisting of the amino acid sequence as defined by SEQ ID NO:40 is called VHH AK-G04, GSDMD5 or VHH-5.

The single-domain antibody of the present invention can also be characterized by being a variant of a single-domain antibody of the present invention characterized by comprising the amino acid sequence as defined in SEQ ID NO: 40. The variant is characterized by comprising an amino acid sequence, wherein the amino acid sequence is at least 80%, 90%, 95%, or 99% identical to the amino acid sequence as defined in SEQ ID NO: 40. In a preferred embodiment, the variant comprises the CDR regions as defined in SEQ ID NOS: 34, 36, and 38, and the variation occurs in the frame work regions.

### GSDMD6 and Variants

In one embodiment, a single-domain antibody of the present invention is characterized by comprising CDR1 as defined by SEQ ID NO:42, CDR2 as defined by SEQ ID NO:44, and CDR3 as defined by SEQ ID NO: 46. Additionally, the single-domain antibody can comprise FR1 as defined by SEQ ID NO:41, FR2: as defined by SEQ ID NO:43, FR3 as defined by SEQ ID NO:45, and FR4 as defined by SEQ ID NO: 47.

The single-domain antibody of the present invention can also be characterized by comprising the amino acid sequence as defined in SEQ ID NO: 48, which consists of SEQ NOS: 41 to 47. The single-domain antibody of the present invention consisting of the amino acid sequence as defined by SEQ ID NO:48 is called VHH AK-G08, GSDMD6 or VHH-6.

The single-domain antibody of the present invention can also be characterized by being a variant of a single-domain antibody of the present invention characterized by comprising the amino acid sequence as defined in SEQ ID NO: 48. The variant is characterized by comprising an amino acid sequence, wherein the amino acid sequence is at least 80%, 90%, 95%, or 99% identical to the amino acid sequence as defined in SEQ ID NO: 48. In a preferred embodiment, the variant comprises the CDR regions as defined in SEQ ID NOS: 42, 44, and 46, and the variation occurs in the frame work regions.

### GSDMD7 and Variants

In one embodiment, a single-domain antibody of the present invention is characterized by comprising CDR1 as defined by SEQ ID NO:50, CDR2 as defined by SEQ ID NO:52, and CDR3 as defined by SEQ ID NO: 54. Additionally, the single-domain antibody can comprise FR1 as defined by SEQ ID NO:49, FR2: as defined by SEQ ID NO:51, FR3 as defined by SEQ ID NO:53, and FR4 as defined by SEQ ID NO: 55.

The single-domain antibody of the present invention can also be characterized by comprising the amino acid sequence as defined in SEQ ID NO: 56, which consists of SEQ NOS: 49 to 55. The single-domain antibody of the present invention consisting of the amino acid sequence as defined by SEQ ID NO:56 is called GSDMD7 or LS-01-A02.

The single-domain antibody of the present invention can also be characterized by being a variant of a single-domain antibody of the present invention characterized by comprising the amino acid sequence as defined in SEQ ID NO: 56. The variant is characterized by comprising an amino acid sequence, wherein the amino acid sequence is at least 80%, 90%, 95%, or 99% identical to the amino acid sequence as defined in SEQ ID NO: 56. In a preferred embodiment, the variant comprises the CDR regions as defined in SEQ ID NOS: 50, 52, and 54, and the variation occurs in the frame work regions.

### GSDMD8 and Variants

In one embodiment, a single-domain antibody of the present invention is characterized by comprising CDR1 as defined by SEQ ID NO:58, CDR2: as defined by SEQ ID NO:60, and CDR3 as defined by SEQ ID NO: 62. Additionally, the single-domain antibody can comprise FR1 as defined by SEQ ID NO:57, FR2: as defined by SEQ ID NO:59, FR3 as defined by SEQ ID NO:61, and FR4 as defined by SEQ ID NO: 63.

The single-domain antibody of the present invention can also be characterized by comprising the amino acid sequence as defined in SEQ ID NO: 64, which consists of SEQ NOS: 57 to 63. The single-domain antibody of the present invention consisting of the amino acid sequence as defined by SEQ ID NO:64 is called GSDMD8 or LS-01-D09.

The single-domain antibody of the present invention can also be characterized by being a variant of a single-domain antibody of the present invention characterized by comprising the amino acid sequence as defined in SEQ ID NO: 64. The variant is characterized by comprising an amino acid sequence, wherein the amino acid sequence is at least 80%, 90%, 95%, or 99% identical to the amino acid sequence as defined in SEQ ID NO: 64. In a preferred embodiment, the variant comprises the CDR regions as defined in SEQ ID NOS: 58, 60, and 62, and the variation occurs in the frame work regions.

### GSDMD9 and Variants

In one embodiment, a single-domain antibody of the present invention is characterized by comprising CDR1 as defined by SEQ ID NO:66, CDR2: as defined by SEQ ID NO:68, and CDR3 as defined by SEQ ID NO: 70. Additionally, the single-domain antibody can comprise FR1 as defined by SEQ ID NO:65, FR2: as defined by SEQ ID NO:67, FR3 as defined by SEQ ID NO:69, and FR4 as defined by SEQ ID NO: 71.

The single-domain antibody of the present invention can also be characterized by comprising the amino acid sequence as defined in SEQ ID NO: 72, which consists of SEQ NOS: 65 to 71. The single-domain antibody of the present invention consisting of the amino acid sequence as defined by SEQ ID NO:72 is called GSDMD9 or LS-01-F06.

The single-domain antibody of the present invention can also be characterized by being a variant of a single-domain antibody of the present invention characterized by comprising the amino acid sequence as defined in SEQ ID NO: 72. The variant is characterized by comprising an amino acid sequence, wherein the amino acid sequence is at least 80%, 90%, 95%, or 99% identical to the amino acid sequence as defined in SEQ ID NO: 72. In a preferred embodiment, the variant comprises the CDR regions as defined in SEQ ID NOS: 66, 68, and 70, and the variation occurs in the frame work regions.

### GSDMD10 and Variants

In one embodiment, a single-domain antibody of the present invention is characterized by comprising CDR1 as defined by SEQ ID NO:74, CDR2: as defined by SEQ ID NO:76, and CDR3 as defined by SEQ ID NO: 78. Additionally, the single-domain antibody can comprise FR1 as defined by SEQ ID NO:73, FR2: as defined by SEQ ID NO:75, FR3 as defined by SEQ ID NO:77, and FR4 as defined by SEQ ID NO: 79.

The single-domain antibody of the present invention can also be characterized by comprising the amino acid sequence as defined in SEQ ID NO: 80, which consists of SEQ NOS: 73 to 79. The single-domain antibody of the present invention consisting of the amino acid sequence as defined by SEQ ID NO:80 is called GSDMD10 or LS-02-A07.

The single-domain antibody of the present invention can also be characterized by being a variant of a single-domain antibody of the present invention characterized by comprising the amino acid sequence as defined in SEQ ID NO: 80. The variant is characterized by comprising an amino acid sequence, wherein the amino acid sequence is at least 80%, 90%, 95%, or 99% identical to the amino acid sequence as defined in SEQ ID NO: 80. In a preferred embodiment, the variant comprises the CDR regions as defined in SEQ ID NOS: 74, 76, and 78, and the variation occurs in the frame work regions.

### GSDMD11 and Variants

In one embodiment, a single-domain antibody of the present invention is characterized by comprising CDR1 as defined by SEQ ID NO:82, CDR2: as defined by SEQ ID NO:84, and CDR3 as defined by SEQ ID NO: 86. Additionally, the single-domain antibody can comprise FR1 as defined by SEQ ID NO:81, FR2: as defined by SEQ ID NO:83, FR3 as defined by SEQ ID NO:85, and FR4 as defined by SEQ ID NO: 87.

The single-domain antibody of the present invention can also be characterized by comprising the amino acid sequence as defined in SEQ ID NO: 88, which consists of SEQ NOS: 81 to 87. The single-domain antibody of the present invention consisting of the amino acid sequence as defined by SEQ ID NO:88 is called GSDMD11 or LS-02-A12.

The single-domain antibody of the present invention can also be characterized by being a variant of a single-domain antibody of the present invention characterized by comprising the amino acid sequence as defined in SEQ ID NO: 88. The variant is characterized by comprising an amino acid sequence, wherein the amino acid sequence is at least 80%, 90%, 95%, or 99% identical to the amino acid sequence as defined in SEQ ID NO: 88. In a preferred embodiment, the variant comprises the CDR regions as defined in SEQ ID NOS: 82, 84, and 86, and the variation occurs in the frame work regions.

### GSDMD12 and Variants

In one embodiment, a single-domain antibody of the present invention is characterized by comprising CDR1 as defined by SEQ ID NO:90, CDR2: as defined by SEQ ID NO:92, and CDR3 as defined by SEQ ID NO: 94. Additionally, the single-domain antibody can comprise FR1 as defined by SEQ ID NO:89, FR2: as defined by SEQ ID NO:91, FR3 as defined by SEQ ID NO:93, and FR4 as defined by SEQ ID NO: 95.

The single-domain antibody of the present invention can also be characterized by comprising the amino acid sequence as defined in SEQ ID NO: 96, which consists of SEQ NOS: 89 to 95. The single-domain antibody of the present invention consisting of the amino acid sequence as defined by SEQ ID NO:96 is called GSDMD12 or LS-02-B10.

The single-domain antibody of the present invention can also be characterized by being a variant of a single-domain antibody of the present invention characterized by comprising the amino acid sequence as defined in SEQ ID NO: 96. The variant is characterized by comprising an amino acid sequence, wherein the amino acid sequence is at least 80%, 90%, 95%, or 99% identical to the amino acid sequence as defined in SEQ ID NO: 96. In a preferred embodiment, the variant comprises the CDR regions as defined in SEQ ID NOS: 90, 92, and 94, and the variation occurs in the frame work regions.

### GSDMD13 and Variants

In one embodiment, a single-domain antibody of the present invention is characterized by comprising CDR1 as defined by SEQ ID NO:98, CDR2: as defined by SEQ ID NO:100, and CDR3 as defined by SEQ ID NO: 102. Additionally, the single-domain antibody can comprise FR1 as defined by SEQ ID NO:97, FR2: as defined by SEQ ID NO:99, FR3 as defined by SEQ ID NO:101, and FR4 as defined by SEQ ID NO: 103.

The single-domain antibody of the present invention can also be characterized by comprising the amino acid sequence as defined in SEQ ID NO: 104, which consists of SEQ NOS: 97 to 103. The single-domain antibody of the present invention consisting of the amino acid sequence as defined by SEQ ID NO:104 is called GSDMD13 or LS-02-C09.

The single-domain antibody of the present invention can also be characterized by being a variant of a single-domain antibody of the present invention characterized by comprising the amino acid sequence as defined in SEQ ID NO: 104. The variant is characterized by comprising an amino acid sequence, wherein the amino acid sequence is at least 80%, 90%, 95%, or 99% identical to the amino acid sequence as defined in SEQ ID NO: 104. In a preferred embodiment, the variant comprises the CDR regions as defined in SEQ ID NOS: 98, 100, and 102, and the variation occurs in the frame work regions.

### GSDMD14 and Variants

In one embodiment, a single-domain antibody of the present invention is characterized by comprising CDR1 as defined by SEQ ID NO:106, CDR2: as defined by SEQ ID NO:108, and CDR3 as defined by SEQ ID NO: 110. Additionally, the single-domain antibody can comprise FR1 as defined by SEQ ID NO:105, FR2: as defined by SEQ ID NO:107, FR3 as defined by SEQ ID NO:109, and FR4 as defined by SEQ ID NO: 111.

The single-domain antibody of the present invention can also be characterized by comprising the amino acid sequence as defined in SEQ ID NO: 112, which consists of SEQ NOS: 105 to 111. The single-domain antibody of the present invention consisting of the amino acid sequence as defined by SEQ ID NO:112 is called GSDMD14 or LS-02-C10.

The single-domain antibody of the present invention can also be characterized by being a variant of a single-domain antibody of the present invention characterized by comprising the amino acid sequence as defined in SEQ ID NO: 112. The variant is characterized by comprising an amino acid sequence, wherein the amino acid sequence is at least 80%, 90%, 95%, or 99% identical to the amino acid sequence as defined in SEQ ID NO: 112. In a preferred embodiment, the variant comprises the CDR regions as defined in SEQ ID NOS: 106, 108, and 110, and the variation occurs in the frame work regions.

### GSDMD15 and Variants

In one embodiment, a single-domain antibody of the present invention is characterized by comprising CDR1 as defined by SEQ ID NO:114, CDR2: as defined by SEQ ID NO:116, and CDR3 as defined by SEQ ID NO: 118. Additionally, the single-domain antibody can comprise FR1 as defined by SEQ ID NO:113, FR2: as defined by SEQ ID NO:115, FR3 as defined by SEQ ID NO:117, and FR4 as defined by SEQ ID NO: 119.

The single-domain antibody of the present invention can also be characterized by comprising the amino acid sequence as defined in SEQ ID NO: 120, which consists of SEQ NOS: 113 to 119. The single-domain antibody of the present invention consisting of the amino acid sequence as defined by SEQ ID NO:120 is called GSDMD15 or LS-02-D10.

The single-domain antibody of the present invention can also be characterized by being a variant of a single-domain antibody of the present invention characterized by comprising the amino acid sequence as defined in SEQ ID NO: 120. The variant is characterized by comprising an amino acid sequence, wherein the amino acid sequence is at least 80%, 90%, 95%, or 99% identical to the amino acid sequence as defined in SEQ ID NO: 120. In a preferred embodiment, the variant comprises the CDR regions as defined in SEQ ID NOS: 114, 116, and 118, and the variation occurs in the frame work regions.

### GSDMD16 and Variants

In one embodiment, a single-domain antibody of the present invention is characterized by comprising CDR1 as defined by SEQ ID NO:122, CDR2: as defined by SEQ ID NO:124, and CDR3 as defined by SEQ ID NO: 126. Additionally, the single-domain antibody can comprise FR1 as defined by SEQ ID NO:121, FR2: as defined by SEQ ID NO:123, FR3 as defined by SEQ ID NO:125, and FR4 as defined by SEQ ID NO: 127.

The single-domain antibody of the present invention can also be characterized by comprising the amino acid sequence as defined in SEQ ID NO: 128, which consists of SEQ NOS: 121 to 127. The single-domain antibody of the present invention consisting of the amino acid sequence as defined by SEQ ID NO:128 is called GSDMD16 or SN-62-E01.

The single-domain antibody of the present invention can also be characterized by being a variant of a single-domain antibody of the present invention characterized by comprising the amino acid sequence as defined in SEQ ID NO: 128. The variant is characterized by comprising an amino acid sequence, wherein the amino acid sequence is at least 80%, 90%, 95%, or 99% identical to the amino acid sequence as defined in SEQ ID NO: 128. In a preferred embodiment, the variant comprises the CDR regions as defined in SEQ ID NOS: 122, 124, and 126, and the variation occurs in the frame work regions.

### GSDMD17 and Variants

In one embodiment, a single-domain antibody of the present invention is characterized by comprising CDR1 as defined by SEQ ID NO:130, CDR2: as defined by SEQ ID NO:132, and CDR3 as defined by SEQ ID NO: 134. Additionally, the single-domain antibody can comprise FR1 as defined by SEQ ID NO:129, FR2: as defined by SEQ ID NO:131, FR3 as defined by SEQ ID NO:133, and FR4 as defined by SEQ ID NO: 135.

The single-domain antibody of the present invention can also be characterized by comprising the amino acid sequence as defined in SEQ ID NO: 136, which consists of SEQ NOS: 129 to 135. The single-domain antibody of the present invention consisting of the amino acid sequence as defined by SEQ ID NO:136 is called GSDMD17 or SN-62-E02.

The single-domain antibody of the present invention can also be characterized by being a variant of a single-domain antibody of the present invention characterized by comprising the amino acid sequence as defined in SEQ ID NO: 136. The variant is characterized by comprising an amino acid sequence, wherein the amino acid sequence is at least 80%, 90%, 95%, or 99% identical to the amino acid sequence as defined in SEQ ID NO: 136. In a preferred embodiment, the variant comprises the CDR regions as defined in SEQ ID NOS: 130, 132, and 134, and the variation occurs in the frame work regions.

### GSDMD18 and Variants

In one embodiment, a single-domain antibody of the present invention is characterized by comprising CDR1 as defined by SEQ ID NO:138, CDR2: as defined by SEQ ID NO:140, and CDR3 as defined by SEQ ID NO: 142. Additionally, the single-domain antibody can comprise FR1 as defined by SEQ ID NO:137, FR2: as defined by SEQ ID NO:139, FR3 as defined by SEQ ID NO:141, and FR4 as defined by SEQ ID NO: 143.

The single-domain antibody of the present invention can also be characterized by comprising the amino acid sequence as defined in SEQ ID NO: 144, which consists of SEQ NOS: 137 to 143. The single-domain antibody of the present invention consisting of the amino acid sequence as defined by SEQ ID NO:144 is called GSDMD18 or SN-62-F05.

The single-domain antibody of the present invention can also be characterized by being a variant of a single-domain antibody of the present invention characterized by comprising the amino acid sequence as defined in SEQ ID NO: 144. The variant is characterized by comprising an amino acid sequence, wherein the amino acid sequence is at least 80%, 90%, 95%, or 99% identical to the amino acid sequence as defined in SEQ ID NO: 144. In a preferred embodiment, the variant comprises the CDR regions as defined in SEQ ID NOS: 138, 140, and 142, and the variation occurs in the frame work regions.

### GSDMD19 and Variants

In one embodiment, a single-domain antibody of the present invention is characterized by comprising CDR1 as defined by SEQ ID NO:146, CDR2: as defined by SEQ ID NO:148, and CDR3 as defined by SEQ ID NO: 150. Additionally, the single-domain antibody can comprise FR1 as defined by SEQ ID NO:145, FR2: as defined by SEQ ID NO:147, FR3 as defined by SEQ ID NO:149, and FR4 as defined by SEQ ID NO: 151.

The single-domain antibody of the present invention can also be characterized by comprising the amino acid sequence as defined in SEQ ID NO: 152, which consists of SEQ NOS: 145 to 151. The single-domain antibody of the present invention consisting of the amino acid sequence as defined by SEQ ID NO:152 is called GSDMD19 or SN-62-F09.

The single-domain antibody of the present invention can also be characterized by being a variant of a single-domain antibody of the present invention characterized by comprising the amino acid sequence as defined in SEQ ID NO: 152. The variant is characterized by comprising an amino acid sequence, wherein the amino acid sequence is at least 80%, 90%, 95%, or 99% identical to the amino acid sequence as defined in SEQ ID NO: 152. In a preferred embodiment, the variant comprises the CDR regions as defined in SEQ ID NOS: 146, 148, and 150, and the variation occurs in the frame work regions.

### GSDMD20 and Variants

In one embodiment, a single-domain antibody of the present invention is characterized by comprising CDR1 as defined by SEQ ID NO:154, CDR2: as defined by SEQ ID NO:156, and CDR3 as defined by SEQ ID NO: 158. Additionally, the single-domain antibody can comprise FR1 as defined by SEQ ID NO:153, FR2: as defined by SEQ ID NO:155, FR3 as defined by SEQ ID NO:157, and FR4 as defined by SEQ ID NO: 159.

The single-domain antibody of the present invention can also be characterized by comprising the amino acid sequence as defined in SEQ ID NO: 160, which consists of SEQ NOS: 153 to 159. The single-domain antibody of the present invention consisting of the amino acid sequence as defined by SEQ ID NO:160 is called GSDMD20 or SN-62-G04.

The single-domain antibody of the present invention can also be characterized by being a variant of a single-domain antibody of the present invention characterized by comprising the amino acid sequence as defined in SEQ ID NO: 160. The variant is characterized by comprising an amino acid sequence, wherein the amino acid sequence is at least 80%, 90%, 95%, or 99% identical to the amino acid sequence as defined in SEQ ID NO: 160. In a preferred embodiment, the variant comprises the CDR regions as defined in SEQ ID NOS: 154, 156, and 158, and the variation occurs in the frame work regions.

### GSDMD21 and Variants

In one embodiment, a single-domain antibody of the present invention is characterized by comprising CDR1 as defined by SEQ ID NO:162, CDR2: as defined by SEQ ID NO:164, and CDR3 as defined by SEQ ID NO: 166. Additionally, the single-domain antibody can comprise FR1 as defined by SEQ ID NO:161, FR2: as defined by SEQ ID NO:163, FR3 as defined by SEQ ID NO:165, and FR4 as defined by SEQ ID NO: 167.

The single-domain antibody of the present invention can also be characterized by comprising the amino acid sequence as defined in SEQ ID NO: 168, which consists of SEQ NOS: 161 to 167. The single-domain antibody of the present invention consisting of the amino acid sequence as defined by SEQ ID NO:168 is called GSDMD21 or SN-62-G11.

The single-domain antibody of the present invention can also be characterized by being a variant of a single-domain antibody of the present invention characterized by comprising the amino acid sequence as defined in SEQ ID NO: 168. The variant is characterized by comprising an amino acid sequence, wherein the amino acid sequence is at least 80%, 90%, 95%, or 99% identical to the amino acid sequence as defined in SEQ ID NO: 168. In a preferred embodiment, the variant comprises the CDR regions as defined in SEQ ID NOS: 162, 164, and 166, and the variation occurs in the frame work regions.

### GSDMD22 and Variants

In one embodiment, a single-domain antibody of the present invention is characterized by comprising CDR1 as defined by SEQ ID NO:170, CDR2: as defined by SEQ ID NO:172, and CDR3 as defined by SEQ ID NO: 174. Additionally, the single-domain antibody can comprise FR1 as defined by SEQ ID NO:169, FR2: as defined by SEQ ID NO:171, FR3 as defined by SEQ ID NO:173, and FR4 as defined by SEQ ID NO: 175.

The single-domain antibody of the present invention can also be characterized by comprising the amino acid sequence as defined in SEQ ID NO: 176, which consists of SEQ NOS: 169 to 175. The single-domain antibody of the present invention consisting of the amino acid sequence as defined by SEQ ID NO:176 is called GSDMD22 or SN-62-G12.

The single-domain antibody of the present invention can also be characterized by being a variant of a single-domain antibody of the present invention characterized by comprising the amino acid sequence as defined in SEQ ID NO: 176. The variant is characterized by comprising an amino acid sequence, wherein the amino acid sequence is at least 80%, 90%, 95%, or 99% identical to the amino acid sequence as defined in SEQ ID NO: 176. In a preferred embodiment, the variant comprises the CDR regions as defined in SEQ ID NOS: 170, 172, and 174, and the variation occurs in the frame work regions.

### GSDMD23 and Variants

In one embodiment, a single-domain antibody of the present invention is characterized by comprising CDR1 as defined by SEQ ID NO:178, CDR2: as defined by SEQ ID NO:180, and CDR3 as defined by SEQ ID NO: 182. Additionally, the single-domain antibody can comprise FR1 as defined by SEQ ID NO:177, FR2: as defined by SEQ ID NO:179, FR3 as defined by SEQ ID NO:181, and FR4 as defined by SEQ ID NO: 183.

The single-domain antibody of the present invention can also be characterized by comprising the amino acid sequence as defined in SEQ ID NO: 184, which consists of SEQ NOS: 177 to 183. The single-domain antibody of the present invention consisting of the amino acid sequence as defined by SEQ ID NO:184 is called GSDMD23 or SN-62-H06.

The single-domain antibody of the present invention can also be characterized by being a variant of a single-domain antibody of the present invention characterized by comprising the amino acid sequence as defined in SEQ ID NO: 184. The variant is characterized by comprising an amino acid sequence, wherein the amino acid sequence is at least 80%, 90%, 95%, or 99% identical to the amino acid sequence as defined in SEQ ID NO: 184. In a preferred embodiment, the variant comprises the CDR regions as defined in SEQ ID NOS: 178, 180, and 182, and the variation occurs in the frame work regions.

### Epitope of the Single-Domain Antibodies of the Present Invention

Preferably, the single-domain antibody of the present invention is directed at an epitope located within the N-terminal domain of GSDMD. The N-terminal domain of GSDMD is involved in oligomerization. Therefore, a single-domain antibody of the present invention binding an epitope located within the N-terminal domain of GSDMD can inhibit the capability of GSDMD to oligomerize, and thereby to form pores.

The single-domain antibody directed against GSDMD of the present invention can be capable of specifically binding an epitope within the N-terminal domain of GSDMD, wherein the epitope is a discontinuous epitope defined by amino acids L16, H18, G19, E21, F22, Q75, G77, R78, F80, S122, S124, D126, P127, N128, Q131, E162, R217, L231, and F232 of sequence of GSDMD (SEQ ID NO:185):

MGSAFERVVRRVVQELDHGGEFIPVTSLQSSTGFQPYCLVVRKPSSSWF
WKPRYKCVNLSIKDILEPDAAEPDVQRGRSFHFYDAMDGQIQGSVELAA
PGQAKIAGGAAVSDSSSTSMNVYSLSVDPNTWQTLLHERHLRQPEHKVL
QQLRSRGDNVYVVTEVLQTQKEVEVTRTHKREGSGRFSLPGATCLQGEG
QGHLSQKKTVTIPSGSTLAFRVAQLVIDSDLDVLLFPDKKQRTFQPPAT
GHKRSTSEGAWPQLPSGLSMMRCLHNFLTDGVPAEGAFTEDFQGLRAEV
ETISKELELLDRELCQLLLEGLEGVLRDQLALRALEEALEQGQSLGPVE
PLDGPAGAVLECLVLSSGMLVPELAIPVVYLLGALTMLSETQHKLLAEA
LESQTLLGPLELVGSLLEQSAPWQERSTMSLPPGLLGNSWGEGAPAWVL
LDECGLELGEDTPHVCWEPQAQGRMCALYASLALLSGLSQEP.

The present invention is also concerned with a polynucleotide encoding the single-domain antibody of the present invention.

The present invention is also concerned with a polynucleotide comprising one or more nucleic acid sequence(s) encoding the single-domain antibody of the present invention. The polynucleotide of the present invention can be selected from RNA, such as mRNA, DNA, such as genomic DNA, cDNA, or synthetic DNA, analogs thereof, or a combination thereof. Preferably the polynucleotide is mRNA.

The polynucleotide of the present invention can be used to transfect a cell by a method as known in the art. For example, AAV-Mediated Gene Therapy can be used. Adeno-associated virus (AAV) is a small, nonenveloped virus that was adapted 30 years ago for use as a gene transfer vehicle. It is capable of transducing a wide range of species and tissues in vivo with no evidence of toxicity, and it generates relatively mild innate and adaptive immune responses. Upon transfection, the cell will produce the single-domain antibody of the present invention. The single-domain antibody thus produced is capable of binding to cytosolic GSDMD in a cell, and thereby inhibiting pyroptosis of the cell. Therefore, the present invention is also concerned with a single-domain antibody, which is capable of binding to cytosolic GSDMD in a cell, and thereby inhibiting pyroptosis of the cell, wherein the single-domain antibody directed against GSDMD is produced by the cell upon transfecting the cell with the nucleic acid encoding the single-domain antibody of the present invention.

The present invention is also concerned with a host cell comprising the polynucleotide encoding the single-domain antibody of the present invention, wherein optionally the host cell is selected from eukaryotic cells, which include, but are not limited to those e.g. hamster cell lines (CHO and their derivatives), mouse cell lines (such as C127, NS0, SP2/0, YB2/0, XB2/09 and derivatives of all of them), or human cell lines (such as HEK and their derivatives, e.g. EXP1293, HT-1080, PER.C6, or HuH-7). Also included are cell lines from monkeys, such as e.g., Vero cells and their derivatives and insect cells, such as SF-9 cells and their derivatives. The polynucleotide encoding the single-domain antibody of the present invention can also be produced recombinantly in bacterial cell, or in yeast cells, such as in Pichia pastoris.

The present invention is also concerned with a pharmaceutical composition comprising the single-domain antibody of the present invention or the polynucleotide encoding the single-domain antibody of the present invention, and a pharmaceutically acceptable carrier.

The single-domain antibodies of the present invention can be capable of preventing oligomerization of GSDMD. As oligomerization of GSDMD leads to pore formation and ultimately pyroptosis of the cell, preventing oligomerization of GSDMD can be a therapeutic approach to stop or prevent pyroptosis and the conditions and symptoms associated with pyroptosis. Therefore, the present invention is also concerned with the single-domain antibodies directed against GSDMD of the present invention or the polynucleotide encoding the single-domain antibodies of the present invention or the pharmaceutical composition of the present invention for use in therapy.

In particular, the present invention is concerned with the single-domain antibodies directed against GSDMD of the present invention or the polynucleotide encoding the single-domain antibody of the present invention or the pharmaceutical composition of the present invention for use in a method of treating or preventing an inflammatory disease or condition in a subject, wherein the inflammatory disease or condition is selected from the group comprising an acute inflammation, a chronic inflammation, sepsis, loss of the blood-brain barrier, in particular caused by sepsis, septic shock, non-alcoholic steatohepatitis, lung cancer, Familial Mediterranean Fever (FMF), autoinflammatory diseases, Cryoprin associated periodic syndrome (CAPS), non-alcoholic fatty liver disease, Alzheimer's disease, Parkinson's disease, age related macular degeneration, atherosclerosis, asthma and allergy airway inflammation, gout, Crohn's disease, ulcerative colitis, inflammatory bowel disease, hypertension, nephropathy, myocardial infarction, multiple sclerosis, experimental autoimmune encephalitis, hyperinflammation following influenza infection, graft versus host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury.

In a preferred embodiment, an mRNA encoding the antibody of the present invention is used in the method of treatment. The mRNA can be taken up by the cells, and the cells will produce the single-domain antibody of the present invention. The single-domain antibody can then prevent oligomerization, or further oligomerization of GSDMD monomers, e.g. in the cytosol of the cell. Existing pores will be repaired my membrane shedding and/or internalization. Thereby, the process leading to pyroptosis can be stopped and/or prevented. In another embodiment, the single-domain antibody itself can be used in the method of treatment. In both cases, uptake of the nucleic acid or the single-domain antibody will be enhanced in cells, which already display pores formed by oligomerization of GSDMD. This is the case because the pores provide easier entry of extracellular molecules. Therefore, cells already displaying pores will take up the single-domain antibody externally provided, or the nucleic acid encoding the single-domain antibody externally provided, more rapidly and in an enhanced amount compared to cells not displaying pores. This allows targeting of affected cells, which provides a better treatment efficacy and less adverse effects.

The single-domain antibodies of the present invention can be suitable for determining, whether oligomerization of GSDMD has taken place in a cellular system.

Therefore, the present invention is also concerned with a method for determining the presence or absence of GSDMD oligomers in a sample obtained from a subject, wherein the method comprises contacting the sample with a reporter system comprising two single-domain antibodies directed against GSDMD, wherein both single-domain antibodies compete for the same epitope in GSDMD, wherein binding of the single-domain antibodies to the epitope does not affect the capability of GSDMD to oligomerize, and wherein upon binding of both single-domain antibodies to GSDMD, the reporter system provides a reporter signal that can be detected indicating the presence of GSDMD oligomers.

In the present invention, the sample can be selected from the group comprising serum, plasma, and whole blood.

The method of the present invention for determining the presence or absence of GSDMD oligomers in a sample obtained from a subject can be sandwich ELISA, wherein the first single-domain antibody directed against GSDMD can be used as primary antibody to capture GSDMD, and the second single-domain antibody directed against GSDMD can be used as a secondary antibody, wherein the secondary antibody is labelled.

The method of the present invention for determining the presence or absence of GSDMD oligomers in a sample obtained from a subject can also be an HTRF/FRET based assay, wherein a first fluorescently labelled single-domain antibody directed against GSDMD can be used as fluorescence donor, and a second fluorescently labelled single-domain antibody directed against GSDMD can be used as fluorescence acceptor.

Examples for the diagnostic method of the present invention are disclosed schematically in FIG. 19.

In the diagnostic method of the present invention, the first single-domain antibody directed against GSDMD and the second single-domain antibody directed against GSDMD can be the single-domain antibody of the present invention, wherein the first single-domain antibody and the second single-domain antibody can be the same single-domain antibody or different single-domain antibodies.

When it is determined that GSDMD oligomers are present in a sample obtained from a subject, it can be concluded that a pyroptotic process is ongoing. In these subjects, a therapeutic approach would be most beneficial, as the therapeutic single-domain antibodies of the present invention are capable of stopping or preventing pyroptosis. Therefore, the present invention is also concerned with a single-domain antibody directed against gasdermin D (GSDMD) or a polynucleotide encoding the single-domain antibody or the pharmaceutical composition of the present invention for use in a method of treating or preventing an inflammatory disease or condition in a subject, wherein the inflammatory disease or condition is selected from the group comprising an acute inflammation, a chronic inflammation, sepsis, septic shock, non-alcoholic steatohepatitis, lung cancer, Familial Mediterranean Fever (FMF), autoinflammatory diseases, Cryoprin associated periodic syndrome (CAPS), non-alcoholic fatty liver disease, Alzheimer's disease, Parkinson's disease, age related macular degeneration, atherosclerosis, asthma and allergy airway inflammation, gout, Crohn's disease, ulcerative colitis, inflammatory bowel disease, hypertension, nephropathy, myocardial infarction, multiple sclerosis, experimental autoimmune encephalitis, hyperinflammation following influenza infection, graft versus host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury, wherein a presence of GSDMD oligomers has been determined in a sample obtained from the subject.

### Definitions

The term “comprise/s/ing”, as used herein, is meant to include or encompass the disclosed features and further features which are not specifically mentioned. The term “comprise/es/ing” is also meant in the sense of “consist/s/ing of” the indicated features, thus not including further features except the indicated features. Thus, the subject-matter of the present invention may be characterized by additional features in addition to the features as indicated.

The term “single-domain antibody” as used herein is interchangeable with the term “nanobody”, and defines a recombinant, antigen-specific antibody consisting of only one single monomeric variable antibody domain (normally, these correspond to the variable region (VHH) of a heavy-chain antibody). Nanobodies can be derived from naturally occurring heavy chain antibodies. Due to their small size they offer several advantages over conventional antibodies.

Sequence identity can be determined by the skilled person. For example, the sequence identity can be calculated using BLASTP as disclosed in the prior art (see e.g. Altschul et al. (1997) “Gapped BLAST and PSI-BLAST: a new generation of protein database search programs”, Nucleic Acids Res. 25:3389-3402; Altschul et al. (2005) “Protein database searches using compositionally adjusted substitution matrices.” FEBS J. 272:5101-5109), preferably using version BLASTP 2.2.29+(http://blast.ncbi.nlm.nih.gov/Blast.cgi), preferably using the following settings:
Field “Enter Query Sequence”: Query subrange: none
Field “Choose Search Set”: Database: non-redundant protein sequences (nr); optional parameters: none
Field “Program Selection”: Algorithm: blastp (protein-protein BLAST)
Algorithm parameters: Field “General parameters”: Max target sequences: 100; Short queries: Automatically adjust parameters for short input sequences; Expect threshold: 10; Word size: 3; Max matches in a query range: 0
Algorithm parameters: Field “Scoring parameters”: Matrix: BLOSUM62; Gap Costs: Existence: 11 Extension: 1; Compositional adjustments: Conditional compositional score matrix adjustment
Algorithm parameters: Field “Filters and Masking”: Filter: none; Mask: none.

Results are filtered for sequences with more than 35% query coverage.

Preferably, the variants can comprise one or more conservative substitutions for amino acids comprised in the exemplary sequences SEQ ID NO: 8, 16, 24, 32, 40, 48, 56, 64, 72, 80, 88, 96, 104, 112, 120, 128, 136, 144, 152, 160, 168, 176, or 184.

A “conservative substitution” refers to the substitution of one amino acid by another, wherein the replacement results in a silent alteration. This means that one or more amino acid residues within the amino acid sequence of the present invention can be substituted by another amino acid of a similar polarity which acts as a functional equivalent. Substitutes for an amino acid within the sequence may be selected from other members of the class to which the amino acid belongs (i.e. a conservative substitution). For example, one polar amino acid can be substituted by another polar amino acid, one positively or negatively charged amino acid, respectively, can be substituted by another positively or negatively charged amino acid, respectively, et cetera. Classes of amino acids are for example, nonpolar (hydrophobic) amino acids including alanine, leucine, isoleucine, valine, proline, phenylalanine, tryptophan and methionine; polar neutral amino acids including glycine, serine, threonine, cysteine, tyrosine, asparagine, and glutamine; positively charged (basic) amino acids including arginine, lysine and histidine; negatively charged (acidic) amino acids including aspartic acid and glutamic acid.

As it is used herein “derivative” and “derivatives” is to be understood as all descendant cell lines that have been derived from them or have emerged from them with modification or further development. Polypeptide expression using cellular systems can be performed by using diverse transfection systems. Non-limiting examples are for example lipid-based transfection or viral transduction techniques, which are very well known to a skilled person in the art.

The patient or subject of the present invention can be a mammal, preferably a human.

These therapeutic and prophylactic aspects of the present invention are preferably achieved by administering an effective amount of the single-domain antibody of the present invention, or the polynucleotide encoding the single-domain antibody of the present invention, or the pharmaceutical composition of the present invention, for a time and under conditions sufficient to appropriately achieve the therapeutic or prophylactic effect.

A “therapeutically effective amount” means an amount that is effective in prevention and/or therapy, or an amount sufficient to provide a preventive and/or therapeutic effect. An amount that is effective in therapy is an amount which produces a biological activity and will depend, among other things, on the individual. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce or retard the severity or progression of an existing condition.

Administration of the single-domain antibody of the present invention, or the polynucleotide encoding the single-domain antibody of the present invention, or the pharmaceutical composition of the present invention may be effected by different ways of administration. Non-limiting examples include, but are not limited to, for example, intravenous, intra-arterial, intraperitoneal, intramuscular, pulmonal, inhalative administration. The dosage regimen will be determined by the attending physician and other clinical factors. As well known the skilled person in art, the dosages for any one patient can vary and depend on many factors, including for example size, age, sex, time and router of administration and stage of the disease.

The invention is further explained by the attached figures and examples, which are intended to illustrate, but not to limit the present invention.

### FIGURES

FIG. 1 shows identification of GSDMD-specific nanobodies. (A) Scheme of GSDMD nanobody generation and selection by phage display. (B) Sequence alignment of the 6 GSDMD-specific nanobodies, with indication of the Complementarity Determining Regions (CDRs). (C) SUMO-GSDMD or control protein SUMO were immobilized to ELISA plates and binding of indicated concentration of HA-His-tagged VHHs quantified by ELISA with anti-HA HRP. (D) LUMIER assay: HEK 293T cells were co-transfected with expression vectors for the the specified HA-tagged VHHs and the indicated protein-Renilla luciferase fusions. 24 h post transfection, cell lysates were generated and VHH-HA immunoprecipitated with immobilized anti-HA. Coelerentazine was added and luminescence of co-purified Renilla luciferase measured and normalized to luminescence of lysates. Data represent average values (with individual data points) from three independent experiments±SEM.

FIG. 2 shows that VHH_{GSDMD-1} and VHH_{GSDMD-2} abrogate pyroptotic cell death. (A) HEK293T cells were co-transfected with expression vectors for the indicated HA-tagged nanobodies and GSDMDNT or the empty vectors. LDH release was measured 24 h post transfection and normalized to cells lysed in Triton X-100 (n=4). (B-E) PMA-differentiated THP-1 macrophages constitutively expressing the indicated HA-tagged nanobodies or wt controls were stimulated with 0.1 μg/mL LFn-MxiH and 1.0 μg/mL PA (MxiH) for 1 h to activate NLRC4 (B,D), or with 200 ng/mL ultrapure LPS for 3 h and 10 μM nigericin (Nig) for 1 h to activate NLRP3 (C,E), in the presence of 40 μM VX-765 (VX) or 2.5 μM CRID3 where indicated. We next measured LDH release as in A (B,C) or IL-1β secretion by Homogeneous Time Resolved Fluorescence (HTRF) (D,E). (F, G) PMA-differentiated THP-1 macrophages were stimulated with NLRC4 and NLRP3 activators as described above, but in the presence of 100 nM DRAQ7. DRAQ7 uptake was monitored over 5 h in an Incucyte Live-Cell Imaging system. Representative images after 1 h (F) and graphs of normalized DRAQ uptake (n=3) are displayed. Scale bar: 100 μm. (H) Overview of transduction of primary human macrophages with lentiviral vectors encoding the different nanobodies and C1C-EGFP under the control of a bidirectional doxycycline (dox)-inducible promoter. Stimulation with NLRC4 activator MxiH triggers cell death by pyroptosis, unless the expressed nanobodies inhibits GSDMD pore formation, which leads to the enrichment of the respective transduced (C1C-EGFP-positive) cells. (I-K) Primary M-CSF-differentiated monocyte-derived human macrophage were transduced with lentiviral vectors packaging Vpx-Vpr and encoding C1C-EGFP and the indicated nanobody. 24 h post transduction, gene expression was induced with dox and 24 h later, cells were treated with NLRC4 activator MxiH as in B and D. 1 h post treatment, cells were harvested, fixed, and analyzed by flow cytometry to determine cell count over 30 sec (1), the fraction of C1C-EGFP+ and thus VHH-expressing cells (J). and the fraction of C1C-EGFP+ cells assembling ASC specks (K). Data represent average values (with individual data points) from three independent experiments or donors±SEM, unless mentioned otherwise.

FIG. 3 shows that VHH_{GSDMD-1} and VHH_{GSDMD-2} prevent oligomerization of GSDMD^{NT} but still allow membrane localization. (A) THP-1 cell lines expressing C1C-mCherry (dox-inducible) as well as the indicated VHH-EGFP fusions (constitutively) were differentiated with PMA, treated with dox for 24 h, and subjected to stimulation with NLRC4 agonist MxiH for 1 h as described in 2A, but in presence of 40 μM VX. Cells were harvested and ASC specks quantified by flow cytometry. Data represent average values (with individual data points) from three independent experiments±SEM. (B,C) PMA-differentiated THP-1 macrophages expressing the indicated HA-tagged nanobodies were stimulated with MxiH for 1 h as described in 2A. Cells were lysed in SDS-PAGE buffer with 100 mM DTT (B) or no reducing agent (C), and subjected to SDS-PAGE and immunoblot with GSDMD and GAPDH antibodies. Representative immunoblot of at least three independent experiments are displayed. (D) HEK 293T cell stably expressing the indicated VHH-EGFP fusions were transfected with expression vectors for GSDMDNT-mCherry I104N (left) or GSDMD-mCherry I104N (right) and analyzed by live confocal imaging. Data representative of three independent experiments with 6 to 22 images per condition is shown. Scale bar=10 μm. (E) PMA-differentiated THP-1 cells constitutively expressing GSDMD-mNeonGreen_ins and the indicated HA-tagged nanobodies were stimulated with MxiH for 1 h as described in 2A. Representative images are displayed. Scale bar=10 μm.

FIG. 4 shows that inhibition of pore formation by VHH_{GSDMD} triggers caspase-1-dependent apoptosis. (A-D) THP-1 cell lines expressing C1C-mCherry (dox-inducible) as well as the indicated VHH-EGFP fusions (constitutively) were differentiated with PMA, treated with dox for 24 h, and stimulated with NLRC4 agonist MxiH for 1 h as described in 2B, with 5 μM staurosporine (stau) to trigger apoptosis. Stimulation was performed in the absence or presence of VX as indicated. (A) Cells representative of three independent experiments were recorded by live confocal microscopy. Scale bar=10 μm. (B) Cells were harvested and analyzed by flow cytometry to quantify C1C-mCherry specks. (C/D) Cells were harvested, stained for cleaved caspase-3, and the fraction of cells positive for cleaved caspase-3 quantified by flow cytometry (C). Representative histograms of two exemplary cell lines with the indicated treatments are presented in (D). (E-L) PMA-differentiated THP-1 wt (E-I, K, L) or THP-1 ΔASC (E, J, K) cells constitutively expressing the indicated HA-tagged nanobodies were stimulated with MxiH for 1 h as described in 2A. Experiments were performed in the presence of 40 μM VX, 50 μM Z-VAD, 30 μM, 20 μM and 4 μM caspase-3/7 inhibitor, or 5/10/20/30 ng/mL perfringolysin O (PFO) as indicated. (E, K) Cells and supernatants were harvested to measure caspase-3/7 (E) or caspase-1 (K, L) activity using Caspase-Glo assays. Activity was normalized to the number of fells using CTB values. Data represent average values (with individual data points) from three independent experiments±SEM. (F-J) Cell lysates were separated by SDS-PAGE and analyzed by immunoblot with the indicated antibodies. Of note, the two caspase-3 blots were developed separately, with a longer exposure for caspase-3cleaved (F). Data represent average values (with individual data points) from three independent experiments±SEM for all flow cytometry and caspase activity assays. Representative immunoblots or microscopy images of at least three independent experiments are displayed.

FIG. 5 shows that recombinant nanobodies inhibit pyroptosis when administered extracellularly. (A-B) PMA-differentiated THP-1 cells were treated with MxiH for 1 h as described in 2A in the presence of increasing concentrations (2, 20, 50, 100, and 200 μg/mL) of the indicated recombinant nanobodies. (A) LDH release was measured and normalized to cells lysed in Triton X-100. (B) IL-1β secretion into the supernatant was quantified by HTRF. (C-D) M-CSF-differentiated primary human macrophages from three independent donors were treated as in 5A/B, and LDH release (C) and IL-1β secretion (D) quantified as before. (E) THP-1 cells treated as in 5A/B in the presence of DRAQ7 were recorded with an Incucyte Live-Cell Imaging system. One representative image is displayed. Red arrows indicate apoptotic cells. Data on LDH and IL-1β release represent average values (with individual data points) from three independent experiments or donors±SEM. (F) shows that fluorescent nanobodies cannot enter intact cells but can indeed enter pyroptotic cells (presumably through GSDMD pores).

FIG. 6 A shows an average distance tree representing the percentage identity between the selected GSDMD-specific nanobodies sequences.

FIG. 6 B shows that inhibition of pore formation by antagonistic GSDMD nanobodies augments caspase-1 activity. PMA-differentiated THP-1 WT cells constitutively expressing the indicated HA-tagged nanobodies were stimulated with MxiH for 1 h as described in FIG. 2A. Experiments were performed in the presence of increasing PFO concentrations (0, 5, 10, 20, 30, 60, 120, 240, and 480 ng/mL). Cells and supernatants were harvested to measure caspase-1 activity using Caspase-Glo assays. Activity was corrected for cell numbers per sample using CTB values and normalized to MxiH-treated cells expressing VHHNP-1 (indicated as dashed line). Data represent average values (with individual data points) from three independent experiments±SEM. ***P<0.001, and ****P<0.0001 (unpaired two-tailed Student's t-test).

FIG. 7 shows that VHH_{GSDMD-1} and VHH_{GSDMD-2} abrogate pyroptosis. (A) THP-1 cell lines constitutively expressing the indicated HA-tagged nanobodies were fixed, stained for HA, and histograms of the HA signals of a representative experiment were displayed. (B-D) Primary GM-CSF-differentiated monocyte-derived human macrophage were transduced and stimulated as described in 2I-K. 1 h post treatment, cells were harvested, fixed, and analyzed by flow cytometry to determine cell count over 30 s (B), the fraction of C1C-EGFP+ and thus VHH-expressing cells (C), and the fraction of C1C-EGFP+ cells assembling ASC specks (D). Data represent average values (with individual data points) from three independent donors±SEM.

FIG. 8 shows that VHH_{GSDMD-1} and VHH_{GSDMD-2} do not interfere with inflammasome assembly. (A) THP-1 cell lines expressing C1C-mCherry (dox-inducible) as well as the indicated VHH-EGFP fusions (constitutively) were differentiated with PMA, treated with dox for 24 h, and stimulated with 200 ng/mL ultrapure LPS for 3 h and 10 μM nigericin (Nig) in presence of VX for 1 h to activate NLRP3. Cells were harvested and ASC specks were quantified by flow cytometry. Data represent average values (with individual data points) from three independent experiments±SEM.

FIG. 9 shows that inhibition of pore formation by antagonistic GSDMD nanobodies augments caspase-1 activity and triggers caspase-1-dependent apoptosis. (A) Primary GM-CSF-differentiated monocyte-derived human macrophages were transduced and stimulated as described in 2I-K. Images of three independent donors were recorded by live confocal microscopy. Scale bar=10 μm. (B-C) PMA-differentiated THP-1 cell lines expressing C1C-mCherry (dox-inducible) as well as the indicated VHH-EGFP fusions (constitutively) were differentiated with PMA, treated with dox for 24 h, and stimulated with 200 ng/mL ultrapure LPS for 3 h and 10 μM nigericin (Nig) for 1 h to activate NLRP3 (B) or with 0.1 μg/mL LFn-MxiH and 1.0 μg/mL PA (MxiH) for 1 h to activate NLRC4 (C). Cells were harvested and analyzed by flow cytometry to quantify C1C-mCherry specks (B), or stained for cleaved caspase-3 for which representative histograms of two control cell lines with the indicated treatments are presented in (C). Experiments displayed in panels B and C were done in parallel to experiments shown in FIG. 4, B-D, but display triggers (B) or individual cell lines (C) that were not displayed in the main figure. (D-E) PMA-differentiated THP-1 ΔASC cells constitutively expressing the indicated HA-tagged nanobodies were stimulated with MxiH for 1 h as described in FIG. 2B. 5 μM staurosporine (Stau) was used as positive control to trigger apoptosis. Stimulation was performed in the absence or presence of VX as indicated. Cells were harvested, stained for cleaved caspase-3, and the fraction of cells positive for cleaved caspase-3 quantified by flow cytometry (D). Representative histograms of the staining for cleaved caspase-3 for indicated treatments are presented in (E). (F-H) PMA-differentiated THP-1 WT or ΔASC cells constitutively expressing the indicated HA-tagged nanobodies were stimulated with MxiH for 1 h as described in FIG. 2B. LDH release was measured and normalized to cells lyzed in Triton X-100 (F). Cells and supernatants were harvested to measure caspase-8 activity using Caspase-Glo assays and activity was corrected for cell numbers per sample using CTB values (G). Cell lysates were separated by SDS-PAGE and analyzed by immunoblot with the indicated antibodies (H). Representative immunoblots of three independent experiments are displayed. Data represent average values (with individual data points) from three independent experiments±SEM, unless otherwise indicated.

FIG. 10 shows identification of six GSDMD targeting nanobodies. a, Amino acid sequence alignment of the GSDMD targeting nanobodies showing the three complementary determining regions (CDR1-3). b, Average distance tree based on the amino acid sequence of the nanobodies. The tree displays the average distance using percent identity and was calculated using the software Jalview. c, Determination of binding affinities using surface plasmon resonance (SPR). Chemically biotinylated GSDMD was immobilized on a sensor chip and nanobodies were injected as analytes at the indicated concentrations for 120 s, followed by dissociation for 300 s. Dissociation constants (K_{Ds}) were determined from the association and dissociation fits by applying a 1:1 binding model. d, Epitope binning assay. Chemically biotinylated GSDMD was immobilized on an SPR sensor chip and the competitive binding of nanobodies was tested in a pairwise manner. Association of the second nanobody to a distinct epitope can be observed as a second association event in the SPR sensorgram. e, Interaction matrix of VHH-1-6. f, Binning of the nanobodies according to their properties in the competitive binding assay.

FIG. 11 shows that VHH-1, VHH-2, and VHH-3 inhibit the formation of functional GSDMD pores in vitro. a, Liposomes composed of POPC, PE, and CL in a 32:55:13 ratio were loaded with the self-quenching dye calcein. GSDMD (or GSDMD-3C) and nanobodies were added in equimolar ratios (0.5 μM). After the addition of 0.2 μM caspase-4 (or 3C protease), calcein release was observed by detecting the fluorescence emitted at 525 nm after excitation at 485 nm. b, GSDMD, nanobodies, and caspase-4 were incubated at 37° C. for 180 min and calcein release was detected every minute. VX-765 was used at 0.125 μM concentration. One representative experiment out of three independent experiments is shown. c, GSDMD-3C, nanobodies, and 3C protease were incubated at 37° C. for 45 min and calcein release was detected every minute. One representative out of three experiments is shown. d, Unfolding temperatures of GSDMD, the nanobodies and GSDMD-nanobody complexes were determined using nanoDSF. The unfolding temperatures of GSDMD at 5 μM and the GSDMD nanobody complexes after addition of 5 μM nanobody are shown. N=x, data represented with SD. e, Unfolding temperatures of GSDMD and GSDMD-nanobody complexes.

FIG. 12 shows the crystal structure of GSDMD in complex with VHH-2 and VHH-6. a, Cartoon representation of the GSDMD-VHH-2-VHH-6 structure consisting of two heterotrimeric complexes. b, Cartoon representation of one of the two heterotrimeric GSDMD-VHH-2-VHH-6 complexes found in the structure. c, SEC-MALS analysis of the GSDMD-VHH-2-VHH-6 complex using an S200 10/300 column. d, Interaction of the N- and C-terminal domains of the two GSDMD molecules. e, Superimposition of the complex of GSDMD NTD and GSDMD′ CTD with the previous GSDMD crystal structure (PDB 6N90.

FIG. 13 shows the interfaces between GSDMD and VHH-2 and VHH-6. a, Cartoon representation of the GSDMD-VHH-2-VHH-6 structure showing GSDMD NTD, GSDMD′ CTD, VHH-2, and VHH-6. CDRs-1, -2, and -3 are highlighted in yellow, orange, and cyan, respectively. b, Electrostatic surface representation of the GSDMD-nanobody complex. The VHH-2 and VHH-6 epitopes are highlighted with dotted lines. c, The GSDMD-VHH-2 interface. Residues directly involved in the interaction are labelled. Salt-bridges and hydrogen-bonds are indicated by dotted lines. d, The GSDMD-VHH-6 interface. As in c, residues directly involved in the interaction are labelled and salt-bridges and hydrogen-bonds are indicated by dotted lines.

FIG. 14 shows the mechanism of pyroptosis inhibition. a, Recombinant GSDMD was incubated with an equimolar amount of VHH-1 or VHH-2 and caspase-4 at 37° C. for 4 h. GSDMD cleavage by caspase-4 was analyzed by SDS-PAGE. b, Superimposition of the nanobody-bound GSDMD NTD with the cryo-EM structure of the activated GSDMD NTD (6VFE).

FIG. 15 shows Nanobodies and GSDMD variants, a, SEC elution chromatogram and b, SDS-PAGE of the recombinantly expressed and purified nanobodies VHH-1 to VHH-6. c, SEC elution chromatogram and SDS-PAGE analysis of wild type, human GSDMD. d, SEC elution chromatogram and SDS-PAGE analysis of a human GSDMD variant (1-484; residues 184-194 and 247-272 were deleted).

FIG. 16 shows SPR-based epitope binning. a, Epitope binning assay with VHH-1 submitted in a first titration step followed by a second titration step with one nanobody of the pool of five (VHH-1, -2, -3, -5, and -6). Chemically biotinylated human, full length GSDMD was immobilized on an SPR sensor chip and the competitive binding of nanobodies was tested in a pairwise manner. b, Epitope binning assay with VHH-2 submitted in the first titration step. Association of the second nanobody to a distinct epitope can be observed for VHH-6 as a second association event in the SPR sensorgram. c-e, Same as in a starting with VHH-3, -5, and -6, respectively.

FIG. 17 shows that VHH-1, VHH-2, and VHH-3 inhibit the formation of functional GSDMD pores in vitro. Replicates of the liposome leakage assay. Data of three independent experiments are represented in individual graphs. Each experiment was carried out in technical replicates and data represented with SEM. a, Leakage assay using GSDMD and caspase-4 for cleavage. b, Leakage assay using GSDMD-3C and the 3C protease for cleavage

FIG. 18 shows the effect of the nanobodies on the thermal stability of GSDMD. a-f, Melting temperatures were determined using nanoDSF. The melting temperatures of GSDMD and the nanobodies alone were determined at concentrations of 5 and 50 μM, respectively. For the titration experiment 5 μM GSDMD was mixed with increasing concentration of the respective nanobody (1-50 μM). g, Summary of melting temperatures

FIG. 19 shows a diagram showing diagnostic methods using the single-domain antibodies of the present invention to determined presence or absence of GSDMD oligomers in a sample. A: GSDMD in the membrane prior and after extraction from membrane; B: Sandwich ELISA scheme; C: HTRF/FRET based assay scheme.

FIG. 20 shows a diagram showing the results of a LUMIER assay: HEK293T cells were co-transfected with expression vectors for the specified HA-tagged nanobodies and the indicated protein-Renilla luciferase fusions. 24 h post transfection, cells were lysed and VHH-HA was immunoprecipitated with immobilized anti-HA. Coelenterazine-h was added and luminescence of co-purified Renilla luciferase was measured and normalized to luminescence of lysates. Data represent average values (with individual data points) from three independent experiments±SEM.

FIG. 21 shows data about the specificity of the nanobodies of the invention. A: VHH_{GSDMD-1} and VHH_{GSDMD-2} inhibit pyroptosis triggered by overexpressed GSDMD^{NT}, but not by its overexpressed related GSDME^{NT}; HEK293T cells were co-transfected with expression vectors for the indicated HA-tagged nanobodies as well as empty vector, GSDME^{NT}. LDH release was measured 24 h post transfection and normalized to cells lysed in 1% Triton X-100 (n=3). B: Partial inhibition of murine GsdmDNT pore formation and uptake of extracellularly administered recombinant antagonistic GSDMD nanobodies. HEK293T cells were co-transfected with expression vectors for the indicated HA-tagged nanobodies as well as empty vector, GSDMD^{NT}, murine GsdmD^{NT} (mGsdmD^{NT}), or GSDME^{NT}. LDH release was measured 24 h post transfection and normalized to cells lysed in 1% Triton X-100. C: Murine iMACs were treated with 200 ng/mL ultrapure LPS for 3 h and 10 μm nigericin (Nig) for 1 h to activate NLRP3 in the presence of increasing concentrations (2, 20, 50, 100, and 200 μg/mL) of the indicated recombinant nanobodies. LDH release was measured and normalized as in B. Data represent average values (with individual data points) from three independent experiments±SEM.

FIG. 22 shows that VHH_{GSDMD-1} and VHH_{GSDMD-2} prevent oligomerization of GSDMD^{NT} but still allow membrane localization of overexpressed GSDMD^{NT}. (A) HEK293T cells stably expressing VHH_{GSDMD}1-EGFP were transfected with expression vectors for the plasma membrane marker emiRFP670-CAAX as well as the indicated GSDMD variants fused to mCherry. Representative images are shown on the top. The distribution of the indicated GSDMD variants in cells positive for emiRFP670 and mCherry were enumerated and average values from three independent experiments with at least n=30 cells per condition are displayed±SEM on the bottom. PM, plasma membrane. Scale bars, 10 μm.

FIG. 23 shows that nanobodies preventing oligomerization still allow membrane localization of processed GSDMD^{NT}. (A,B) PMA-differentiated THP-1 cells constitutively expressing GSDMD-mNeonGreen_ins (GSDMD-mNG_ins) and the indicated HA-tagged nanobodies were stimulated with 1.0 μg/mL PA and 0.1 μg/mL LFn-MxiH (MxiH) for 1 h to activate NLRC4. The plasma membrane of cells in (A) and (B) was stained with CellMask™ Orange (CMO) prior to stimulation. Cells were recorded by live cell confocal microscopy and representative images are displayed (A). Fluorescence intensities along the orange diagonal are displayed to indicate plasma membrane localization of GSDMD^{NT}-mNG (A). The fraction of cells with clear plasma membrane localization of GSDMD^{NT}-mNG was enumerated and average values from three independent experiments with at least n=330 cells per condition are displayed±SEM (B).

FIG. 24 shows that nanobodies preventing oligomerization still allow membrane localization of processed GSDMD^{NT}. PMA-differentiated THP-1 cells constitutively expressing GSDMD-mNG_ins and the indicated HA-tagged nanobodies were stained with CMO, left untreated (left) or stimulated with MxiH (right), and recorded as described for FIG. 23A. Additional representative images and intensity profiles of the experiment in FIG. 23A are displayed. Images representative of at least three independent experiments are displayed. Scale bars, 10 μm.

FIG. 25 shows that nanobodies preventing oligomerization still allow membrane localization of processed GSDMD^{NT}. PMA-differentiated THP-1 cells constitutively expressing VHH_{GSDMD-1}-HA were stimulated with MxiH for 15, 30, and 60 minutes. Cells were fixed, stained with fluorescent wheat germ agglutinin (WGA), fixed again, permeabilized, and stained for cleaved GSDMD^{NT} (anti-GSDMD^{NT}) and DNA (Hoechst 33342). Cells were recorded by confocal microscopy and representative images are displayed (A). Fluorescence intensities along the orange diagonal are displayed to indicate plasma membrane localization of GSDMD^{NT}-mNG. The fraction of cells with clear plasma membrane localization of GSDMD^{NT} was enumerated and average values from three independent experiments with at least n=100 cells per condition are displayed±SEM (B).

FIG. 26 shows GSDMD^{NT}-mNG localizes to internal structures after pore formation. (A, B) PMA-differentiated THP-1 cells constitutively expressing GSDMD-mNG_ins and VHH_{NP-1}-HA were stained with CMO and stimulated with MxiH as in FIG. 24. (A) Stimulated cells were followed over time by live cell confocal microscopy (3 min intervals; time post treatment indicated). Merged images with enhanced brightness are displayed for the later time point on the right. Intracellular vesicular structures positive for GSDMD-mNG and CMO are highlighted with yellow arrows. Images representative of at least three independent experiments are displayed. Scale bars, 10 μm. (B) A Z stack containing a representative cell was recorded 60 min post stimulation. An XY section as well as matching XZ and YZ sections are shown for the cell in the middle.

FIG. 27 shows that inhibition of pore formation by antagonistic GSDMD nanobodies triggers caspase-1-dependent apoptosis, and that extracellular nanobodies inhibit NLRP3-dependent pyroptosis. (A) THP-1 cell lines expressing the indicated HA-tagged VHHs were differentiated with PMA, and were stimulated with 1.0 μg/mL PA and 0.1 μg/mL LFn-MxiH (MxiH) for 1 h to activate NLRC4 in the presence of 100 nM SYTOX Green nucleic acid stain. Cells were recorded by live cell confocal microscopy including bright field recordings. The absolute number of cells positive for SYTOX Green as well as cells with pyroptotic and apoptotic morphology were enumerated per tile scan (675 μm×675 μm) and average values from three independent experiments±SEM are displayed. (B,C) Recombinant antagonistic GSDMD nanobodies inhibit pyroptosis when administered extracellularly. PMA-differentiated THP-1 cells were treated with 200 ng/mL ultrapure LPS for 3 h and 10 μm nigericin (Nig) for 1 h to activate NLRP3 in the presence of increasing concentrations (2, 20, 50, 100, and 200 μg/mL) of the indicated recombinant nanobodies. (B) LDH release was measured and normalized to cells lysed in Triton X-100. (C) IL-1β in the supernatant was quantified by HTRF. Average values from three independent experiments±SEM are displayed. (Data on LDH and IL-1β release represent average values (with individual data points) from three independent experiments or donors±SEM. *P<0.05, **P<0.01, ***P<0.001, and ****P<0.0001 (unpaired two-tailed Student's t-test).

FIG. 28 shows that inhibition of pore formation by antagonistic GSDMD nanobodies triggers caspase-1-dependent apoptosis. (A,B) PMA-differentiated THP-1 cells were treated with MxiH, 100 ng/mL PFO, or 5 μM Stau for 1, 2, 3, 4, and 20 h in the presence of 200 μg/mL of the indicated nanobodies. The reducing capacity as a readout for viability was determined by CellTiter-Blue® (CTB) assay and normalized to untreated cells in the presence of VHH_{NP-1}. As a positive control, cells were incubated with 1% Triton X-100. Where indicated, cells were treated for 4 h in the presence of 40 μM caspase-3/7 inhibitor (casp-3/7i) (B). (Data on CTB assays represent average values (with individual data points) from three independent experiments or donors±SEM. *P<0.05, **P<0.01, ***P<0.001, and ****P<0.0001 (unpaired two-tailed Student's t-test).

FIG. 29 shows that extracellular antagonistic GSDMD nanobodies prevent pyroptosis and enter cells in a caspase-1-dependent manner. (A) M-CSF-differentiated primary human macrophages from independent donors were treated as described previously, and LDH release was quantified as before. (B) THP-1 cells were differentiated with PMA, labeled with CMO, and stimulated with MxiH in the presence of 200 μg/mL VHHNP-1 total (60 μg/mL of the nanobody was AF647 labeled). Where indicated, stimulation was performed in the presence of 40 μM VX (B). Cells were recorded by live cell confocal microscopy including bright field recordings. Cell areas (mostly containing a single cell) were identified using the CMO staining by Imaris. Cell areas were scored as VHH-positive (VHH+) when VHHNP-1-AF647 intensity was at least 80 (corresponding to ca. 75% of the mean intensity outside the cells in the first data set). The fraction of VHH+ cell areas was normalized to the total cell area. Average values from three independent experiments±SEM are displayed. (C) M-CSF-differentiated primary human macrophages from three independent donors cells were treated with MxiH for 1 h as described in 2A in the presence of increasing concentrations (2, 20, 50, 100, and 200 μg/mL) of the indicated recombinant nanobodies. IL-1 secretion into the supernatant was quantified by HTRF. (Data on LDH release and IL-1β secretion represent average values (with individual data points) from three independent experiments or donors±SEM. *P<0.05, **P<0.01, ***P<0.001, and ****P<0.0001 (unpaired two-tailed Student's t-test).

FIG. 30 shows that uptake of extracellularly administered recombinant nanobodies relies on caspase-1 and GSDMD pore formation. (A, B) THP-1 cells (A) or THP-1 cell lines expressing the indicated HA-tagged VHHs (B) were differentiated with PMA, labeled with CMO, and stimulated with MxiH in the presence of fluorescent VHH_{NP-1} as described in FIG. 29B. Cells were recorded by live cell confocal microscopy including bright field recordings and images representative of three independent experiments are displayed. VHH_{NP-1} AF647 in endosomes is indicated with yellow arrows, while cytosolic VHH_{NP-1} AF647 is highlighted with white arrows. Quantification of VHH uptake is displayed in FIG. 29B. Scale bars, 50 μm. NS, not significant (unpaired two-tailed Student's t-test).

FIG. 31 shows that pyroptoptic cells and cells with transient GSDMD pores take up different amounts of DNA dyes. (A-C) PMA-differentiated THP-1 cells were treated with MxiH as described previously, but in the presence of SYTOX Green and 200 μg/mL of the indicated nanobodies in the absence (A, C) or presence (B) of VX. Cells were recorded with an Incucyte Live-Cell Imaging system and the integrated SYTOX Green intensity per cell area displayed over time (A, B). SYTOX Green-positive nuclei were identified and the mean fluorescence intensity was extracted. Nuclei were categorized in the indicated bins of SYTOX Green intensity and the resulting histograms were displayed (C) to compare dye influx in presence of inhibitory VHH_{GSDMD-1} (left) and VHH_{GSDMD-1} (right) to control nanobody VHH_{NP-1} (pyroptotic cells). Data representative of at least three independent experiments is displayed.

FIG. 32 shows that nanobodies are taken up by pyroptotic cells and cells with transient GSDMD pores in a caspase-1 dependent manner. (A-E) PMA-differentiated THP-1 constitutively expressing C1C-EGFP (THP-1^{C1C-EGFP}) were treated with MxiH as described previously in the presence of 30 μg/mL VHH_{ASC} AF647 as well as 200 μg/mL of VHH_{GSDMD-1} (A) or VHH_{NP-1} (B); cells were stimulated in the absence (top) or presence of VX (bottom). Cells were recorded by live cell confocal microscopy and representative images are displayed. Scale bars, 50 μm. Three exemplary cells with assembled inflammasomes (C1C-EGFP specks) are displayed in color-coded insets to the right. Nuclei and C1C-EGFP specks were detected and the fraction of cells with C1C-EGFP specks (C), the fraction of cells with full VHH_{ASC} AF647 uptake throughout the cell (represented by AF647-positive nuclei) (D), as well as the fraction of C1C-EGFP specks positive for AF647 (indicative of minimal VHH uptake) (E) were quantified and average values from three independent experiments with at least n=250 cells per condition (typically more than 500 cells) are displayed±SEM.

### EXAMPLES

### Example 1—Material and Methods

### Cell LINES

Human embryonic kidney (HEK) 293T cells (ATCC Cat #CRL-3216, RRID: CVCL_0063), were cultivated in DMEM GlutaMax™ medium (Gibco) containing 10% FB; THP-1 cells (ATCC TIB-202) were cultured in RPMI 1640 GlutaMax™ medium (Gibco) containing 10% FBS and 50 μM 2-mercaptoethanol. All genetically modified cell lines were generated by lentiviral transduction using lentivirus produced with packaging vectors psPax2 and pMD2.G (kind gifts from Didier Trono, Ecole polytechnique fédérale de Lausanne, Switzerland). THP-1 or HEK293T cell lines constitutively expressing VHH_{GSDMD-1}, VHH_{GSDMD-2}, VHH_{GSDMD-3}, VHH_{NP-1} or VHH_{ASC} under the control of the human elongation factor-1 a promoter (pEF1α) were generated using lentiviral vectors constructed by Gateway cloning (Thermo Fisher Scientific) using vectors modified from pRLL (a kind gift of Susan Lindquist, Whitehead Institute of Biomedical Research), followed by selection in 0.75 μg/mL puromycin (Life Technologies). Cell lines inducibly expressing the C1C-EGFP or C1C-mCherry inflammasome reporter were generated using lentiviruses produced with derivates of plnducer20 followed by selection in 500 μg/mL geneticin (Gibco). These cell lines formed the basis for further lentiviral transduction to incorporate the constitutively expressing nanobodies as described above. THP-1 ΔASC cells expressing EGFP-tagged VHH_{GSDMD-1}, VHH_{GSDMD-2}, VHH_{NP-1} or VHH_{ASC} were generated without subsequent antibiotic selection, but instead were sorted at the BD FacsAria Fusion cell sorter for EGFP positivity as indication for VHH expression. All expression levels were verified by flow cytometry; either by looking at a right shift in case of EGFP-tagged VHHs, C1C-EGFP, or C1C-mCherry, or by a staining using a 1:1000 dilution of the anti-HA antibody (B6 HA.11 Epitope Tag) in combination with a secondary anti-mouse AF-488 (1:500) antibody in case of HA-tagged VHHs. Cells were fixed in 4% formaldehyde and measured at the MACSQuant of BD FACSCanto flow cytometer. Cell lines are routinely tested for Mycoplasma contamination. All experiments involving lentiviruses were conducted in a Biosafety Level 2 laboratory.

### Primary Cells

Human CD14⁺ monocytes were isolated from human whole blood buffy coats obtained from the blood bank of the University hospital of Bonn, with consent of healthy donors and according to protocols accepted by the institutional review board of the University of Bonn (local ethics votes Lfd. Nt. 075/14). The PBMCs were isolated using Ficoll-Paque™ PLUS (VWR) to create a gradient where PBMCs gather between the Ficoll and plasma layer. Positive selection using CD14 (human) MicroBeads (Miltenyi Biotec) separated the CD14⁺ monocytes from the other PBMCs. CD14⁺ monocytes were differentiated into M-CSF or GM-CSF macrophages using 100 ng/mL of recombinant human M-CSF (Immunotools) or 500 U/mL of recombinant human GM-CSF (Immunotools) cytokines for 3 days in RPMI 1640 GlutaMax™ medium supplemented with 10% FBS, PenStrep (500 U/mL), and Sodium Pyruvate (1 mM). To transiently incorporate VHH_{GSDMD-1}, VHH_{GSDMD-2} or VHH_{NP-1} into the primary macrophages, they were transduced for 6 h with a lentivirus expressing both a HA-tagged VHH and the C1C-EGFP inflammasome reporter under a bidirectional doxycycline-inducible promoter derived from the lentiviral vector plnducer20bi-NA, a derivative of plnducer20-NA. In addition, the lentiviruses were constructed using the packaging vectors psPax2 and pMD2.G as described above, as well as a pCAGGs VPx-VPr vector to inhibit SAMHD1 to promote expression in primary human macrophages. The next day, expression of both the VHH and the C1C-EGFP was induced with 1 μg/mL doxycycline for 24 h.

### Proteins

Expression and Purification of his-SUMO-GSDMD and his-SUMO Lfn-MxiH PA

Expression vectors for human His-SUMO-GSMD and His-SUMO were generated by Gibson cloning of GSDMD into the vector pET28. Proteins were expressed in Escherichia (E.) coli LOBSTR cells in Terrific Broth induced with 0.2 or 1 mM IPTG at an OD600 of 0.6. Cells were cultivated for 24 h at 18° C. and lysed by French Press or sonication with a Bandelin Sonopuls HD2070 with TT13 tip. Subsequently, the proteins were purified by Ni-NTA affinity chromatography using Ni-NTA agarose beads (Qiagen) and gel filtration with a HiLoad 16/600 Superdex 75 μg column in buffers containing 20 mM HEPES pH 7.4, 150 mM NaCl, and 10% glycerol.

### Expression and Purification of Nanobodies

Nanobody coding sequences for the different anti-GSDMD VHHs and the control VHHNP-1 were cloned into pHEN6-based bacterial, periplasmic expression vectors with C-terminal LPETG-His6 (large scale) or HA-His6 (small scale) tags using conventional restriction enzyme cloning with NcoI and BstEII. The nanobody expression vectors were transformed into E. coli WK6 and grown in Terrific Broth. Expression was induced with 1 mM IPTG at an OD600 of 0.6, followed by cultivation at 30° C. for 16 h. Bacterial pellets were resuspended in TES buffer (200 mM Tris-HCl pH 8.0, 0.65 mM EDTA, 0.5 M sucrose), after which periplasmic extracts were generated by osmotic shock in 0.25×TES at 4° C. overnight. Eventually, the nanobodies underwent Ni-NTA purification using Ni-NTA agarose beads (Qiagen), followed by gel filtration with a HiLoad 16/600 Superdex 75 μg column in buffers containing 20 mM HEPES pH 7.4, 150 mM NaCl, and 10% glycerol. Nanobodies used for the ELISA experiments were purified in small scale and after the Ni-NTA purification only desalted by PD MiniTrap G-25 columns (GE Healthcare Life Sciences). For the use of the recombinant nanobodies in the culture medium of primary cells, an endotoxin removal procedure was performed using the Pierce™ High Capacity Endotoxin Removal Spin Columns (Thermo Fischer Scientific).

### Antibodies

The following antibodies were used: rabbit polyclonal anti-GSDMD (Atlas Antibodies Cat #HPA044487, RRID:AB_2678957), mouse anti-GAPDH clone 0411 (Santa Cruz Biotechnology Cat #sc-47724, RRID:AB_627678), mouse anti-vinculin clone hVIN-1 (Sigma-Aldrich Cat #V9131, RRID:AB_477629), rabbit anti-caspase-7 clone D2Q3L (Cell Signaling Technology Cat #12827T, RRID:AB_2687912), rabbit anti-PARP clone 46D11 (Cell Signaling Technology Cat #9532S, RRID:AB_659884), rabbit anti-DFNA5/GSDME clone EPR19859 (Abcam Cat #ab215191, RRID:AB_2737000), mouse anti-caspase-8 clone 1C12 (Cell Signaling Technology Cat #9746S, RRID:AB_2275120), rabbit anti-caspase-3 clone D3R6Y (Cell Signaling Technology Cat #14220, RRID:AB_2798429), mouse anti-caspase-9 clone C9 (Cell Signaling Technology Cat #9508S, RRID:AB_2068620), rabbit polyclonal anti-BID (Cell Signaling Technology Cat #2002S, RRID:AB_10692485), goat polyclonal anti-rabbit IgG (H+L)-HRP (Invitrogen Cat #31460, RRID:AB_228341), goat polyclonal anti-mouse IgG (H+L)-HRP (Invitrogen Cat #31430, RRID:AB_228307), mouse anti-HA-HRP clone 6E2 (Cell Signaling Technology Cat #2999S, RRID:AB_1264166), mouse anti-HA. 11 Epitope tag clone 16B12 (BioLegend Cat #901503, RRID:AB_2565005), goat polyclonal anti-mouse IgG (H+L)-Alexa Fluor™ 488 (Invitrogen Cat #A-11029, RRID:AB_2534088), rabbit anti-cleaved caspase-3 (Asp175) clone 5A1E (Cell Signaling Technology Cat #9664S, RRID:AB_2070042), goat polyclonal anti-rabbit IgG (H+L)-Alexa Fluor™ Plus 647 (Invitrogen Cat #A32733, RRID:AB_2633282), rabbit polyclonal anti-E-tag-HRP (Bethyl Cat #A190-133P, RRID:AB_345222).

### Small Compound Inhibitors and Reagents

The following small compound inhibitors and reagents were used: caspase-3/7 inhibitor I (Sigma), CRID3 (MCC-950) (Tocris), doxycycline (Biomol), LPS-EK Ultrapure (Invivogen), MG-132 (Selleckchem), N-ethylmaleimide (NEM) (Sigma), Nigericin sodium salt (Biomol), PMA (phorbol 12-myristate 13-acetate) (Sigma), Roche cOmplete™ Mini protease Inhibitor Cocktail (Sigma Aldrich), staurosporine (Enzo), Vx-765/belnacasan (Selleckchem), Z-VAD(Ome)-FMK (MedChemExpress).

### Nanobody Library Generation

To raise heavy chain-only antibodies (VHHs) against human GSDMD, an alpaca was immunized four with 200 μg GSDMD using Imject™ Alum Adjuvant (Thermo Fisher Scientific) according to locally authorized protocols. The VHH plasmid library in the M13 phagemid vector pD (pJSC) were generated as described before (Schmidt et al., 2016b, Koenig et al 2021). In brief, RNA from peripheral blood lymphocytes was extracted and used as a template to generate cDNA using three sets of primers (random hexamers, oligo(dT), and primers specific for the constant region of the alpaca heavy chain gene). VHH coding sequences were amplified by PCR using VHH-specific primers, cut with AscI and NotI, and ligated into an M13 phagemid vector (pJSC) linearized with the same restriction enzymes. E. coli TG1 cells (Agilent) were electroporated with the ligation reactions and the obtained ampicillin-resistant colonies were harvested, pooled, and stored as glycerol stocks.

### Nanobody Identification Using VHH Phage Display and Panning

GSDMD-specific VHHs were obtained by phage display and panning. E. coli TG1 cells containing the VHH library were infected with helper phage VCSM13 to produce phages displaying the encoded VHHs as pIII fusion proteins. Phages in the supernatant were purified and concentrated by precipitation. Phages presenting GSDMD-specific VHHs were enriched using biotinylated GSDMD immobilized on Dynabeads™ MyOne™ Streptavidin T1 (Life Technologies). The retained phages were used to infect E. coli ER2738 and subjected to a second round of panning. 96 E. coli ER2837 colonies yielded in the second panning were grown in 96-well plates and VHH expression was induced with IPTG. VHHs leaked into the supernatant were tested for specificity using ELISA plates coated with control protein SUMO or SUMO-GSDMD. Bound VHHs were detected with HRP-coupled rabbit anti-E-Tag antibodies (1:10,000), and the chromogenic substrate tetramethylbenzidine (TMB) (Life Technologies). Reactions were stopped with 1 M HCl and absorption at 450 nm was recorded using a SpectraMax i3 instrument and the SoftMax Pro 6.3 Software (Molecular Devices). Positive candidates were sequenced and representative nanobodies were cloned into bacterial expression vectors for further analysis.

### Nanobody ELISA

To test nanobody candidates, SUMO-GSDMD or SUMO in PBS were immobilized on ELISA plates at a concentration of 1 μg/mL overnight. Subsequently, the immobilized antigens were incubated with the HA-tagged nanobodies in 10% FBS/PBS in a 10-fold dilution series ranging from 100 nM to 1 pM. The nanobodies were detected using the mouse anti-HA HRP antibody (1:5000) and developed using the chromogenic substrate TMB. The reaction was stopped using 0.5M HCl, after which the absorption was measured at 450 nm using a SpectraMax i3 instrument and the SoftMax Pro 6.3 Software (Molecular Devices).

### LUMIER Assay

To test the functionality of our VHHs in the reducing environment of the cellular cytosol, a LUMIER assay was performed. HEK293T cells (2.5·10⁵ cells per well in a 24-well plate, seeded the day before) were co-transfected with 0.25 μg pCAGGS VHH-HA expression vectors and 0.25 μg of the Renilla-fused bait proteins GSDMD, GSDMD^{4A}, GSDMD^{NT-4A} GSDMD^{CT} or the control NLRP1^{CARD} using PEI Max (Polysciences). A Lumitrac 600 white high-binding 96-well plate (Labomedic) was coated with 20 μg/mL of the mouse anti-HA.11 Epitope tag clone 16B12 antibody in PBS. The next day, the transfected HEK293T cells were lysed in LUMIER lysis buffer (50 mM Hepes-KOH pH 7.9, 150 mM NaCl, 2 mM EDTA, 0.5% Triton X-100, 5% glycerol and Roche cOmplete™ Mini protease Inhibitor Cocktail) and administered to both the HA-coated Lumitrac plate as well as an uncoated Lumitrac plate as lysate control. Upon addition of the Renilla luciferase substrate coelenterazine-h, the luminescence was measured for both the immunoprecipitated (IP) lysates as well as the lysate control using a SpectraMax i3 instrument and the SoftMax Pro 6.3 Software (Molecular Devices). The values plotted are the IP luminescence values normalized by the values of the lysate.

### Cell Death Quantification by LDH Release

To quantify pyroptotic cell death, PMA-differentiated (50 μg/mL, 18 h followed by 24 h resting period) THP-1 cells or M-CSF differentiated primary human macrophages in presence of intracellularly expressed or extracellularly added recombinant VHHs were seeded (3·10⁵ cells per well in a 24-well plate) and the NLRP3 and/or the NLRC4 inflammasome was activated. NLRP3 is activated using 10 μM nigericin (Nig), which is a potassium ionophore derived from Streptomyces hygroscopicus, in OptiMEM for 1 h. It is preceded by priming for 3 h using 200 ng/mL ultrapure LPS, in presence or absence of 40 μM VX or 2.5 μM CRID3 which are added 30 minutes before stimulation as well as with the stimulus. The NLRC4 inflammasome is activated using recombinant MxiH fused to the N-terminal domain of anthrax lethal factor (LFn-MxiH) (0.1 μg/mL) in OptiMEM that can be delivered to the cytosol by exploiting the channel formed by protective antigen (PA) (1.0 μg/mL), also in presence or absence of 40 μM VX. The extracellular administration of recombinant VHHs in increasing concentrations (1, 2, 20, 50, 100 and 200 μg/mL) occurred simultaneously with the inflammasome stimulus. To measure pyroptotic cell death in HEK293T cells, the cells (5·10⁵ cells per well in a 24-well plate) were co-transfected for 4 h with 0.50 μg HA-tagged VHH and 0.25 μg GSDMD^{NT} or empty vector using Lipofectamine™ 2000 (L2000) Transfection Reagent (Invitrogen). The supernatants were collected 24 h after the transient transfection and subjected to a lactate dehydrogenase (LDH) release assay using the LDH Cytotoxicity Detection kit (TaKaRa or Roche) according to the manufacturer's instructions. Absorption at 492 nm was measured using a SpectraMax i3 instrument and the SoftMax Pro 6.3 Software (Molecular Devices). Control samples, in which cells were lysed in 1% Triton X-100, were used to normalize LDH release, after subtraction of medium background signal.

### Cytokine Quantification by HTRF

To quantify IL-1β secretion, the supernatants obtained via procedures as described above for the LDH release assay were subjected to a human IL-1β Homogeneous Time Resolved Fluorescence (HTRF) assay (Cisbio) according to the manufacturer's instructions. Emissions at 616 nm and 665 nm were measured after excitation at 340 nm using a SpectraMax i3 instrument and IL-1β levels were calculated by the SoftMax Pro 6.3 Software (Molecular Devices) based on the standard curve.

### Cell Death Quantification by DRAQ7 Uptake

To quantify cell death over time, 4·10⁴ PMA-differentiated THP-1 cells per well were seeded in a 96-well plate and treated as described above for the LDH assay. The stimulus medium was complemented with the non-cell permeable DNA dye DRAQ7 (1:3000) (Biolegend) and the uptake was analyzed using the Incucyte Live-Cell Imaging system (Sartorius). The cells were imaged every 5 minutes for a total of 5 h using the Incucyte SX5 instrument, taking 4 images per well. The number of DRAQ7-positive nuclei (cell death count) and the cell confluency were analyzed using the Incucyte 2021C software. For every single image, the cell death count was corrected by subtraction of the value at the start of the experiment. The corrected cell death count was further normalized to the cell confluency by division, before the average value for all 4 images was calculated and plotted over time.

### Flow Cytometry-Based Quantification

To quantify the amount of successfully transduced non-pyroptotic primary human (G)M-CSF macrophages upon lentiviral transduction with the EGFP-tagged C1C inflammasome reporter and the HA-tagged VHHs, the fraction of EGFP⁺ cells was quantified by flow cytometry. To at the same time compare the amount of non-pyroptotic cells per sample, the measurements took place for a fixed time period of 30 s. The reduction in cellular amounts measured is an indirect indication for pyroptotic cell death, since only pyroptotic cells do not survive the flow cytometry procedure and can therefore not be measured. Furthermore, the recruitment of C1C-EGFP to ASC specks was measured by gating for the EGFP⁺ population by the lower width and higher height signal as described elsewhere. For these procedures, 1·10⁵ primary macrophages were treated in 24-wells and lentivirally transduced as described above. The NLRC4 inflammasome was activated using 0.1 μg/mL LFn-MxiH in combination with 1.0 μg/mL PA for 1 h. The cells were harvested by trypsinization, fixed in 4% formaldehyde and analyzed using the BD FACSCanto flow cytometer.

For the assessment of successful inflammasome formation in presence of the EGFP-tagged VHHs, the PMA-differentiated THP-1 macrophages expressing both VHH-EGFP and C1C-mCherry were stimulated with either 0.1 μg/mL LFn-MxiH in combination with 1.0 μg/mL PA or 200 ng/mL LPS (3 h pre-treatment) and 10 μM nigericin for 1 h. To prevent the loss of responding cells by caspase-1-dependent pyroptosis, the cells were stimulated in the presence of 40 μM VX. The amount of specking C1C-mCherry+ single cells were measured. To also show that the inhibitory anti-GSDMD VHHs induce a specking response without undergoing pyroptosis, the experiment was also performed in absence of VX to allow pyroptotic cell death and cell loss. Staining of cleaved caspase-3 in PMA-differentiated THP-1 macrophages was performed as measure for apoptotic cell death. 3·10⁵ THP-1 macrophages were treated in 24-wells with 0.1 μg/mL LFn-MxiH in combination with 1.0 μg/mL PA for 1 h in presence or absence of 40 μM VX. Staurosporine is a non-selective inhibitor of several kinases and functioned here as a positive control for intrinsic apoptosis and caspase-3 activation. After fixation, the cells were permeabilized and stained with an Intracellular Staining Permeabilization Wash buffer (Biolegend), combined with the rabbit anti-cleaved caspase-3 primary antibody (1:2000) and subsequently the goat anti-rabbit Alexa Fluor™ Plus 647-coupled, highly cross-absorbed secondary antibody (1:500). Single cells expressing the EGFP-tagged VHHs were analyzed for their cleaved caspase-3⁺ cell population using the BD LSRFortessa SORP flow cytometer. All flow cytometry data was analyzed using FlowJo 10.7.1 software.

### Live Cell Confocal Microscopy

For live cell confocal microscopy experiments, 9·10⁴ PMA-differentiated THP-1 cells or 2·10⁴ GM-CSF differentiated primary human macrophages were cultured in a 15 μ-slide 8 well Ibidi chamber or a black, clear bottom, TC treated PhenoPlate™ 96-well microscopy plate (Perkin Elmer), respectively. The NLRC4 inflammasome was activated using 0.1 μg/mL LFn-MxiH and 1.0 μg/mL PA for 1 h in imaging medium (RPMI with 10% FBS, 50 μM 2-mercaptoethanol, 30 mM Hepes, no phenol red). HEK293T cells were seeded on poly-L-lysine-coated (Sigma, mol wt 70,000-150,000) Ibidi chambers (9·10⁴ cells per well) constitutively expressing the EGFP-tagged VHHs. Next, they were transiently transfected with 0.25 μg of the expression vector GSDMD I104N-mcherry or GSDMD^{NT} I104N-mCherry for 5 h. Images were recorded at least every 10 minutes using the HC PL APO CS2 63×1.20 water objective on a Leica SP8 Lightning confocal microscope (37° C., 5% CO2). Images were processed using ImageJ 2.3.0 software.

### Immunoblot

To detect the presence and/or cleavage of our proteins of interest, 3-4·10⁵ (per well in a 24-well plate) or 1.25·10⁶ (per well in a 6-well plate) PMA-differentiated THP-1 cells treated as described above were lysed in 100 μL or 300 μL RIPA buffer (50 mM Tris pH 7.4, 150 mM NaCl, 1% NP-40, 0.25% Na-deoxycholate, 2 mM EDTA, 0.1% SDS, Roche cOmplete™ Mini protease Inhibitor Cocktail), respectively, to generate immunoblot samples. Directly before running the samples, they were supplemented with a final concentration of 1×SDS-PAGE sample buffer (50 mM Tris pH 6.8, 0.01% bromophenol blue, 10% glycerol, 2% SDS) with or without 100 mM DTT, sheared and cooked for 5 minutes at 95° C. Proteins were separated by SDS-PAGE using 10% or 12% self-made SDS-PAGE gels. Separated proteins were transferred to PVDF membranes (0.45 μm, Merck) by semi-dry transfer. All immunoblots were blocked in 5% non-fat dry milk (NFDM) solution in TBS-T (0.05% Tween-20) for 2 h and probed with the following primary antibody dilutions: anti-GSDMD 1:500, anti-GAPDH 1:1000, anti-vinculin 1:1000, anti-caspase-3 1:500, anti-caspase-7 1:500, anti-caspase-8 1:500, anti-caspase-9 1:500, anti-PARP 1:500, anti-GSDME 1:500, anti-BID 1:500. All primary antibodies were added in NFDM solution. After overnight incubation at 4° C., the immunoblots were probed with HRP-coupled secondary antibodies in NFDM solution (1:3000) for 2 h. Chemiluminescent signal was induced by Western Lightning® Plus-ECL (Perkin Elmer), except for immunoblots of GSDMD, caspase-3, caspase-7, caspase-8, caspase-9 and BID which required Western Lightning Ultra (Perkin Elmer). The signal was detected using a Fusion Advancer imaging system (Vilber) and images were taken using the EvolutionCapt SL6 software (Vilber).

### Caspase-Glo Activity and Cell Titer Blue Assay

To quantify the activity of caspase-1, caspase-3/7, and caspase-8, 5·10⁴ PMA-differentiated THP-1 cells cultured in a 96-well plate were activated for 1 h using recombinant LFn-MxiH (0.1 μg/mL) in OptiMEM together with 1.0 μg/mL PA. Next, the cells plus supernatants were combined with an equal volume of the caspase-Glo reagent for 1 h at RT according to the manufacturer's instructions (Promega). The mixture was then added to a Lumitrac plate and the luminescence was measured using a SpectraMax i3 instrument and the SoftMax Pro 6.3 Software (Molecular Devices). The value of the blank control with only OptiMEM combined with caspase-Glo reagent was subtracted from the measured values. In parallel, a cell titer blue (CTB) assay was conducted to determine the viability of the cultured cells using the CellTiter-Blue® Reagent (Promega) according to the manufacturer's instructions. The metabolic activity of viable cells can be measured by the conversion of resazurin to the highly fluorescent resofurin. Fluorescence was measured at 585 nm. Due to the linear relationship between the fluorescence and the cell number, the relative cell numbers per cell line could be deduced. The value for the control well without cells was subtracted and the cell line expressing the control VHHNP-1 was set to 1. The final CTB values were used to correct for discrepancies in cell numbers between the different cell lines in the caspase-Glo assays. For the caspase-1 Glo assay, the values were further normalized against the MxiH-treated VHH52 samples to highlight the increased caspase-1 activity in apoptotic over pyroptotic cells.

### Protein Expression and Purification

The coding sequence for human GSDMD 1-484 was cloned into a pET28-derived bacterial expression vector providing an N-terminal His₆-SUMO-tag. For crystallization experiments, residues 247-272 (GSDMD_{Δ247-272}) or residues 184-194 and 247-272 (GSDMD_{Δ184-194/Δ247-272}) were deleted to prevent precipitation during crystallization. The expression constructs were transformed in E. coli Rosetta (DE3) cells, and the cells were grown in 2x-LB-medium containing 0.5% glucose and 50 μg/ml kanamycin at 37° C. until the OD₆₀₀ reached 0.8. Expression was induced overnight at 20° C. with 0.2 mM isopropyl β-D-1-thiogalactopyranoside IPTG and 0.6% (w/v) lactose. Cells were harvested and lysed by sonication in a lysis buffer containing 25 mM Tris (pH 8.0), 200 mM NaCl, 10% glycerol, 10 mM imidazole, and 5 mM DTT. Cell lysates were cleared by centrifugation at 20,000 rpm for 45 min and the His-SUMO fusion proteins were enriched on Ni-NTA beads (Thermo Fisher Scientific). The protein was eluted using a buffer containing 25 mM Tris (pH 8.0), 200 mM NaCl, 300 mM imidazole, and 5 mM DTT, and a buffer exchange to 25 mM Tris (pH 8.0), 200 mM NaCl, 5% glycerol and 5 mM DTT was performed using a PD10 column (Cytiva). The samples were incubated with the SUMO protease ULP1 (homemade) at 4° C. overnight, followed by a second Ni-NTA chromatography to remove uncleaved protein, ULP1 protease, and the His-SUMO-tag. Subsequently, the samples were subjected to size-exclusion chromatography using an S200 column (GE Healthcare) and a buffer containing 20 mM HEPES (pH 8.0), 200 mM NaCl and 5 mM DTT.

The pHEN6 vectors for bacterial, periplasmic expression of GSDMD-targeting nanobodies were transformed into E. coli WK6 cells. VHH-1, -2, and -3 were expressed with a C-terminal LPTEG-His-tag, and VHH-4, -5, and -6 were expressed with a C-terminal HA-His-tag. Cells were grown at 37° C. in TB medium containing 100 μg/ml ampicillin until the OD₆₀₀ reached 0.6. Expression was induced overnight with 1 mM IPTG at 20° C. Cells were harvested and periplasmic extracts were generated using osmotic shock. For this purpose, cell pellets were resuspended in TES buffer (20 mM Tris (pH 8.0), 0.65 mM EDTA, 0.5 M sucrose) and incubated for 1 h, followed by incubation in 0.25×TES for at least 1 h at 4° C. Lysates were cleared by centrifugation at 20,000 rpm for 45 min and the His-tagged nanobodies were enriched using Ni-NTA beads. Beads were washed using a buffer containing 50 mM Tris (pH 7.5, 150 mM NaCl, and 10 mM imidazole and proteins were eluted in the same buffer supplemented with 0.5 M imidazole. Protein containing elution fractions were pooled and subjected to gel filtration in a buffer containing 20 mM HEPES pH 7.5, and 150 mM NaCl using an S75 16/600 column (GE Healthcare).

The coding sequence for human Caspase-4 was cloned into the pACEBac1-His-SUMO expression vector. Plasmids were amplified in E. coli DH10 cells. Baculoviruses were produced by transfection of the bacmid DNA into Sf9 insect cells, using the transfection reagent cellfectin (Mirus Bio, Madison WI). The transfection was carried out in a 6-well format with 0.7×10⁶ cells/well and cells were incubated at 27° C. After three days, the initial virus stock V₀ was harvested and used to infect a 20 ml culture of Sf9 cells grown to a density of 0.6×10⁶ cells/ml. V₁ viruses were harvested after three days and an Sf9 culture grown to a density of 1.0×10⁶ cells/ml was infected with 2% V₁. After another three days, the V₂ viruses were harvested. Expression cultures were infected at a cell density of 1.5×10⁶ cells/ml with 1% virus and proteins were expressed for 48 h at 27° C. Cells were harvested and lysed by sonication in a buffer containing 50 mM Tris (pH 8.0), 150 mM NaCl, 5 mM imidazole, and 2 mM β-ME. Cell lysates were cleared by centrifugation at 20,000 rpm for 45 min. His-SUMO-caspase-4 was purified using Ni-NTA affinity chromatography. Elution fractions containing the His-SUMO-fusion protein were pooled and concentrated in a 30 K Amicon to a concentration of 10 mg/ml. The sample was incubated at 4° C. overnight to enhance the auto-activation of the protease. For the liposome leakage assay, a GSDMD variant was used in which the caspase cleavage site was mutated to a 3C protease cleavage site (LEVLFQGP instead of LTDGVPAE at position 273-280).

### Protein Crystallization and Data Collection

Screening of crystallization conditions for wild type GSDMD and GSDMD_{Δ184-194/Δ247-272} in complex with nanobodies VHH-1-VHH-6 and the combination of VHH-2 plus VHH-6 was performed using commercial kits from Molecular Dimensions (Maumee, OH, USA) and Jena Bioscience (Jena, Germany) with the sitting drop vapor diffusion method. Initial crystals of the sample containing GSDMD_{Δ184-194/Δ247-272} in complex with VHH-2 and VHH-6 were obtained at a protein concentration of 20 mg/ml using a reservoir solution containing 0.07 M NaCl, 22% (v/v) PEG 400 and 0.05 M Na3Cit pH 4.5 at 20° C. Optimization of crystallization conditions led to well diffracting crystals grown at 20 mg/ml in a reservoir solution consisting of 0.04 M NaCl, 25.8% (v/v) PEG 400 and 0.05 M Na3Cit pH 4.4 at 20° C.

Crystals were frozen in the reservoir solutions plus PEG 400 at a final concentration of 35% in liquid nitrogen. X-ray diffraction data were collected at beamline P13 of the PETRA Ill synchrotron at “Deutsches Elektronen-Synchrotron” (DESY) in Hamburg, Germany, at a wavelength of λ=0.976255 Å. Diffraction data were processed with the program XDS. The phases were determined by molecular replacement. For GSDMD, the previous crystal structure of human GSDMD (PDB: 6N90) was used as a search model. To account for possible movements between the N- and C-terminal domains of GSDMD, the structure was split into the GSDMD-NTD or -CTD, resulting in two separate search models. In addition, the structure of the BC2 nanobody (PDB: 5IVO) was used as a search model for VHH-2 and VHH-6. Manual model building and refinement were performed with Coot and Phenix, respectively. The crystal structures were validated by the MolProbity server. Structure figures were prepared using PyMOL (The PyMOL Molecular Graphics System, Version 2.0 Schrödinger, LLC). The interacting residues in the protein interfaces were determined with PDBePISA (https://www.ebi.ac.uk/pdbe/pisa/). Intermolecular salt bridges and hydrogen bonds are indicated by dashed lines and all residues whose buried surface area contributes with more than 10 Å2 to the interface are labelled. Structure figures were prepared using PyMOL (The PyMOL Molecular Graphics System, Version 2.0 Schrödinger, LLC).

### Surface Plasmon Resonance

Surface plasmon resonance experiments were performed using a Biacore 8K instrument (GE Healthcare). The flow system was cleaned using the maintenance “Desorb” function (Desorb Kit, GE Healthcare). The system was flushed with running buffer (20 mM HEPES (pH 8.0), 200 mM NaCl, 5 mM DTT, 0.05% Tween20) and all steps were performed at 25° C. chip temperature. Chemically biotinylated GSDMD at a concentration of 100 nM was immobilized for 180 s on the flow cell 2 of a Series S Sensor Chip CAP using a biotin capture kit and a flow rate of 30 μl/min. The system was washed for 600 s with running buffer with a flow rate of 30 μl/min. Binding affinities were determined using multi-cycle kinetics. To account for different binding affinities, the nanobodies were injected at various concentrations (VHH-1, -2, -3, -5: 0.5-32 nM, VHH-4, and VHH-6: 64-4096 nM) with a flow rate of 30 μl/min. The association step was carried out for 120 s and the dissociation step for 300 s.

For epitope binning experiments, the nanobodies were pairwise tested for competitive binding. The first analyte was injected at a concentration of 128 nM for 120 s with a flow rate of 10 μl/min. This step was followed by a dissociation step for 60 s. Then, a mixture of the first and the second analyte (both 128 nM) was injected for 120 s with a flowrate of 10 μl/min, followed by a dissociation step of 30 s. After each cycle, surfaces were regenerated for 120 s using the regeneration solution of the capture kit with a flow rate of 10 μl/min. Data were referenced by blank cycle (no analyte injected) and subtraction of the reference flow cell (flow cell 1). Data were analyzed using the Biacore Insight Evaluation Software. Dissociation constants were determined based on fits applying a 1:1 binding model.

### Liposome Leakage Assay

The lipids for the generation of LUVs were obtained from Avanti polar lipids and dissolved in chloroform to a final concentration of 25 mg/ml. Liposomes were generated by mixing 80 μl phosphatidylcholine (POPC), 128 μl phosphatidylethanolamine (POPE), and 64 μl cardiolipin in a glass tube. The chloroform was evaporated under a steady stream of nitrogen and lipids were rehydrated in 400 μl of an 80 mM calcein solution in H₂O (pH 7.0). The liposome suspension was vortexed extensively and subjected to five freeze and thaw cycles followed by extrusion through a 100 nm pore diameter polycarbonate membrane 31-times using the Avanti mini-extruder (Avanti Polar Lipids, Inc., Alabaster, AL). The extruded liposomes were passed through a PD-10 column equilibrated with 20 mM HEPES (pH 7.4), 150 mM NaCl and 1 mM EDTA to remove excess calcein. For this purpose, 100 μl of liposomes were loaded onto the column and 200 μl elution fractions were collected. The homogeneity and quality of obtained liposomes were controlled using DLS and evaluating the packaging by measuring the fluorescence at 525 nm after lysis with 1% Triton-X 100, respectively. Fractions containing liposomes of good quality were pooled and diluted 1:10 in buffer.

For the liposome leakage assay, 120 μl of a liposome solution, 0.5 μM GSDMD, 0.2 μM His-SUMO-caspase-4, and 0.5 μM VHH were mixed in a final volume of 200 μl in a dark-well glass bottom plate and incubated at 37° C. for 180 minutes. In a second experiment, 120 μl of a liposome solution, 0.5 μM GSDMD-3C, 0.2 μM 3C-protease (homemade), and 0.5 μM VHH were mixed in a final volume of 200 μl in a dark-well glass bottom plate and incubated at 37° C. for 45 minutes. Every minute, the fluorescence emitted at 525 nm upon excitation at 485 nm was measured using a plate reader.

### Thermal Shift Assay

NanoDSF was used to determine the effect of the GSDMD-targeting nanobodies on the thermal stability of GSDMD. Samples containing varying concentrations of protein were loaded into glass capillaries and applied to the nanoDSF device Prometheus NT.48 (Nanotemper). The samples were heated from 20 to 90° C. with a slope of 1.5° C./min and the unfolding of the proteins was observed by detecting shifts in the fluorescence at 330 and 350 nm. Data were analyzed using the Nanotemper PR.ThermControl software.

### Multi-Angle Light Scattering (MALS)

For SEC-MALS analysis of the GSDMD-VHH_{GSDMD-2}-VHH_{GSDMD-6}, GSDMD_{Δ184-194/Δ247-272} was mixed with VHH_{GSDMD-2}, VHH_{GSDMD-6} or both nanobodies at equimolar concentrations (188.8 μM) and injected into a Superose 6 10/300 GL column equilibrated with GSDMD-SEC buffer. The chromatography system was attached to a three-angle light scattering detector 380 (miniDAWN, Wyatt) and a refractive index detector (Optilab T-rEX, Wyatt). Data were collected every 0.5 s with a flow rate of 0.5 ml/min and analysed using the ASTRA V software (Wyatt).

### Caspase Cleavage Assay

Recombinant GSDMD (15 μM) was incubated with an equimolar amount of VHH_{GSDMD-1} or VHH_{GSDMD-2} and caspase-4 (6 μM) at 37° C. for 4 h. GSDMD cleavage by caspase-4 was analysed by SDS-PAGE at the indicated time points.

### Inflammasome Activation

To induce the human NLRC4 inflammasome, we employed 1.0 μg/mL Bacillus anthracis protective antigen (PA) to deliver recombinantly purified Shigella flexneri needle protein MxiH fused to B. anthracis LFn (LFn-MxiH, 0.1 μg/mL) into the cytosol for 1 h as described in the art. MxiH binds to human NAIP, which in turn initiates the oligomerization of NLRC4. NLRP3 is an indirect sensor for potassium efflux and perturbations of intracellular homeostasis. To stimulate NLRP3, the cells were primed using 200 ng/mL ultrapure LPS for 3 h and NLRP3 was activated by adding 10 μM nigericin (Nig), a potassium ionophore derived from Streptomyces hygroscopicus, for 1 h. Where indicated, caspase-1 activity was inhibited with 40 μM VX-765, or NLRP3 was inhibited with 2.5 μM CRID3 for 30 min before and during stimulation.

### Cell Death Quantification by CellTiter-Blue Assays (Reducing Activity)

CellTiter-Blue® (CTB) assays were conducted to determine the reducing capacity and thus viability of untreated or stimulated cells using the CellTiter-Blue® Reagent (Promega) according to the manufacturer's instructions. Cells in 96-well plates were treated as for LDH assays. Supernatants were aspirated and replaced with 100 μL of the CTB reagent, followed by incubation at 37° C. for 1 h. Samples were excited with light at a wavelength of 560 nm and fluorescence measured at 585 nm using a SpectraMax i3 instrument.

### Quantification of Expression Levels, Inflammasome Assembly, and Caspase-3 Cleavage by Flow Cytometry

Transduction of primary human M-CSF macrophages with lentiviruses encoding the C1C-EGFP inflammasome reporter and VHH-HA was assessed by quantifying the fraction of cells positive for C1C-EGFP by flow cytometry. To estimate the total number of intact cells per sample, cells were treated identically, resuspended in the same volume, and measured by flow cytometry for a fixed time period of 30 s. The reduction of cells per volume served as an indirect indication for pyroptotic cell death. Caspase-1^{CARD}-EGFP (C1C-EGFP) recapitulates the recruitment of unprocessed caspase-1 to nascent ASC specks by homotypic interactions between caspase-1^{CARD} and ASC^{CARD} and can therefore be used as a fluorescent reporter for ASC speck and thus inflammasome assembly. To quantify C1C-EGFP specks as a proxy for inflammasome assembly, we exploited that the peculiar redistribution of EGFP fluorescence from cytosolic to speck-associated yields a separate population of cells exhibiting higher fluorescence intensity, EGFP(H), and narrower width of the fluorescent signal, EGFP(W). We first gated cells positive for C1C-EGFP [EGFP(A)], and then plotted height against width of the C1C-EGFP signal. For these experiments, 1·10⁵ transduced primary macrophages or PMA-differentiated THP-1 derivatives in 24-wells were stimulated as described above. The cells were harvested by trypsinization, fixed in 4% formaldehyde, and analyzed using a BD FACSCanto flow cytometer. For the quantification of NLRP3 and NLRC4 inflammasome assembly in presence of cytosolic VHH-EGFP fusions, PMA-differentiated THP-1 macrophages expressing both VHH-EGFP and C1C-mCherry were stimulated as described above. To prevent the loss of responding cells by caspase-1-dependent pyroptosis, the cells were stimulated in presence of 40 μM VX. The fraction of specking C1C-mCherry positive cells was measured with a BD LSRFortessa SORP flow cytometer. Experiments were also performed in absence of VX and revealed that pyroptotic cells are lost during sample processing, while untreated and apoptotic cells could be analyzed by flow cytometry. To measure cleaved caspase-3 in PMA-differentiated THP-1 macrophages, we performed experiments as described for apoptotic cell death. 3·10⁵ THP-1 macrophages in wells of 24-well plates were treated with 1.0 μg/mL recombinant B. anthracis PA and 0.1 μg/mL LFn-MxiH for 1 h in presence of 40 μM VX where indicated. Staurosporine is a non-selective inhibitor of several kinases and was added for 20 h as a positive control for intrinsic apoptosis and caspase-3 activation. After fixation, cells were stained with rabbit anti-cleaved caspase-3 primary antibody (1:2000) and goat anti-rabbit Alexa Fluor Plus 647-coupled secondary antibody (1:500). The fraction of cells positive for cleaved caspase-3 was measured with a BD LSRFortessa SORP flow cytometer. All flow cytometry data was analyzed using the FlowJo 10.7.1 software.

### Confocal Microscopy

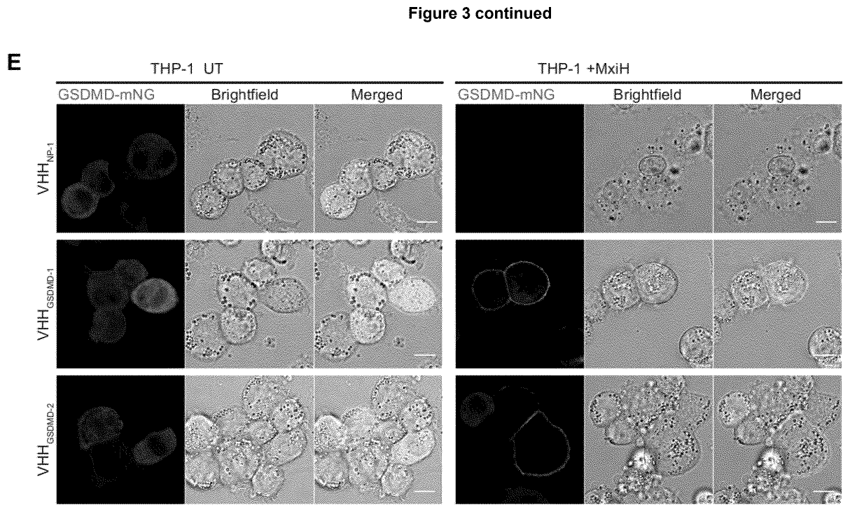

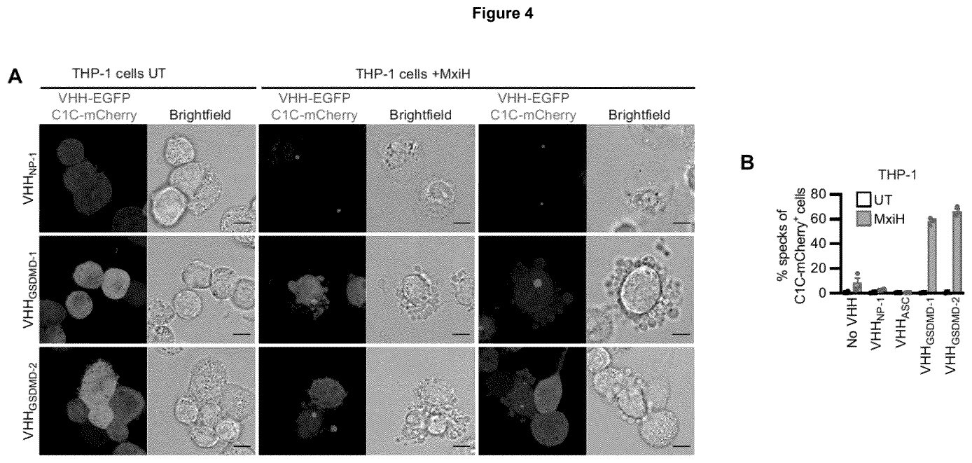

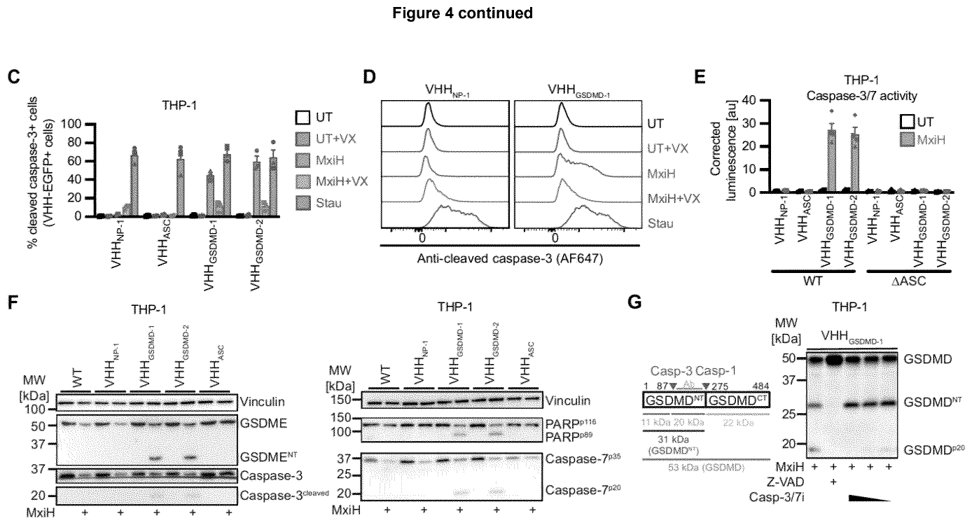

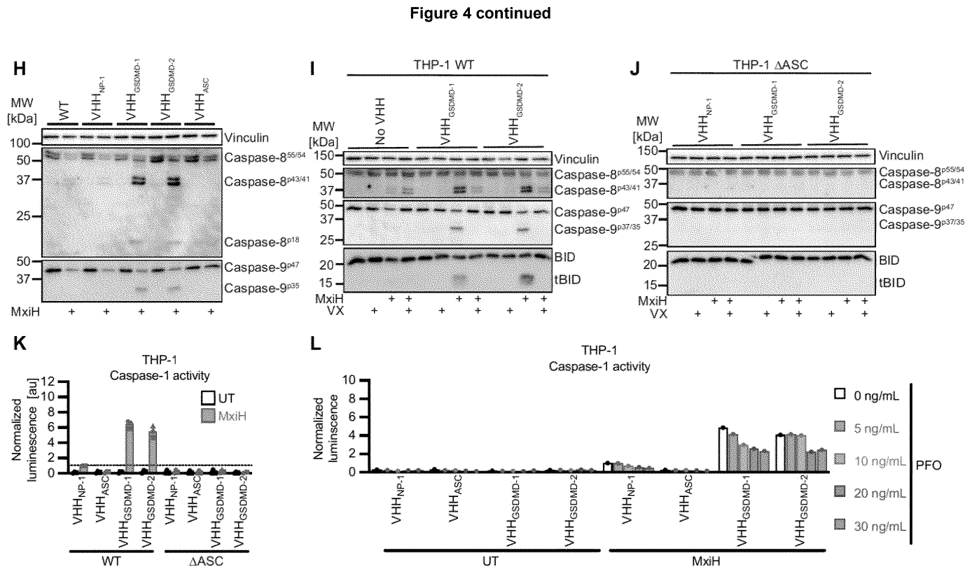

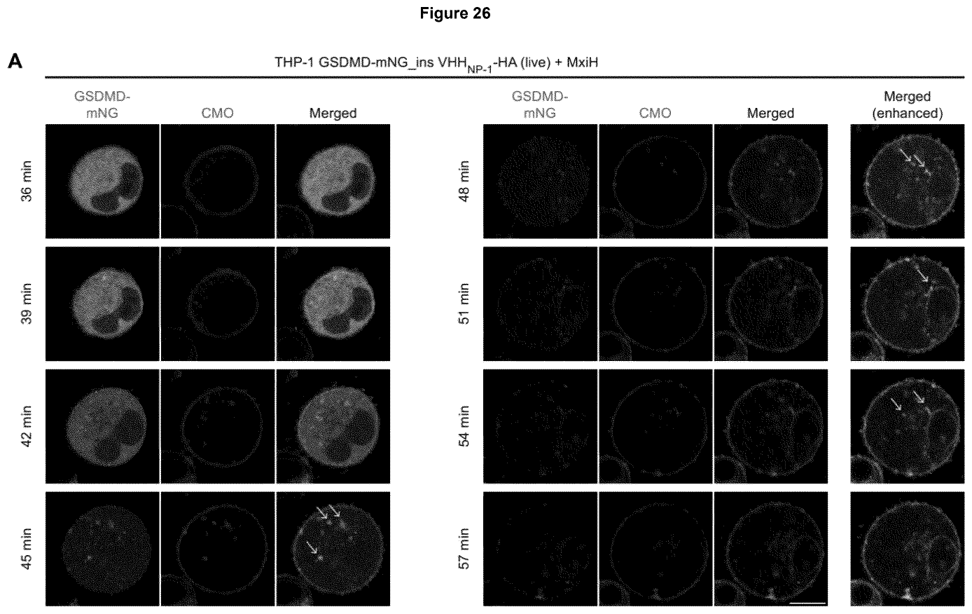

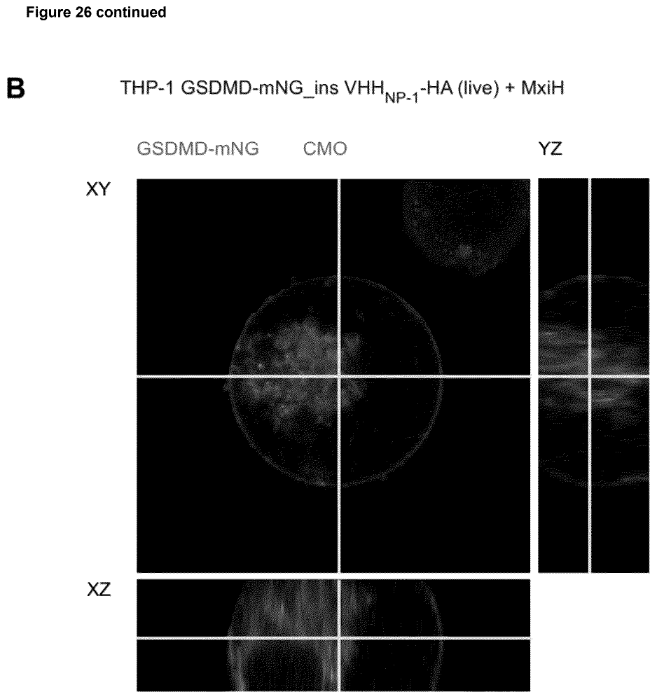

For live cell confocal microscopy experiments, PMA-differentiated THP-1 cells or GM-CSF differentiated primary human macrophages were cultured in 15 μ-slide 8 well Ibidi chambers (9·10⁴ cells) or black, clear bottom, TC treated PhenoPlate™ 96-well microscopy plates (Perkin Elmer) (2-5·10⁴ cells). Where indicated, cells were stained with CellMask™ Orange Plasma membrane stain (1:10,000, Thermo Fisher Scientific) at 37° C. for 10 min, followed by three washes with Opti-MEM. The NLRC4 inflammasome was activated with PA and LFn-MxiH for 1 h in imaging medium (RPMI with 10% FBS, 50 μM 2-mercaptoethanol, 30 mM HEPES, no phenol red) using concentrations as described above. To stain endogenous proteins in microscopy samples, cells were seeded and treated as above, fixed in 4% formaldehyde in PBS for 20 minutes; where indicated, cells were stained with 5 μg/mL WGA AF647 and fixed again. To stain intracellular proteins, cells were permeabilized with 0.5% Triton X-100, and stained with Hoechst 33342 (Thermo Fisher Scientific) as well as rabbit anti-cleaved GSDMD^{NT} antibody (1:500)+goat anti-rabbit IgG AF488 (1:1000), or mouse anti-TOM20 antibody (1:500)+goat anti-mouse IgG AF647 (1:1000) in PBS+10% goat serum as indicated. Most images were recorded with the HC PL APO CS2 63×/1.20NA water objective on a Leica SP8 Lightning confocal microscope. Images in FIG. 4G as well as FIG. 26A, 32, A-C, were recorded with the HC PL APO CS2 63x/1.20NA water objective on a Leica Stellaris 8 microscope. HEK293T cells constitutively expressing VHH-EGFP fusions were seeded in Ibidi chambers (9·10⁴ cells per well) coated with poly-L-lysine (mol wt 70,000-150,000) (Sigma Aldrich). They were transiently transfected with expression vectors for fusions of GSDMD and GSDMD^{NT} variants with mCherry. In initial experiments, we employed the attenuating GSDMD mutant I104N to facilitate the observation of membrane-associated GSDMD^{NT} as described before. As the mutant largely behaved like WT GSDMD in our assays, we use WT GSDMD in later experiments. Where indicated, cells were co-transfected with expression vectors for emiRFP670 with a C-terminal CAAX motif (emiRFP670-CAAX). Emi-RFP670-CAAX is prenylated and anchors the fluorescent protein to the plasma membrane allowing us to assess membrane localization. 5 h post transfection, images were recorded at least every 10 minutes using the HC PL APO CS2 63×1.20 water objective on a Leica SP8 Lightning confocal microscope (370 C, 5% CO2). Alternatively, cells were fixed 12 h post transfection, stained for DNA, and images recorded with the same microscopy setup.

### Image Analysis

Images were processed using ImageJ 2.3.0 software. To quantify the influx of fluorescent nanobodies, we used the cell detection tool of Imaris (Bitplane) to detect cell areas using the CMO channel, mostly containing correctly segmented cells, but occasionally clusters of cells (detection of cells without nucleus or vesicle staining; Cell Type=Cell Membrane; Cell Smallest Diameter=12 μm; Cell Membrane Detail=1 μm; Cell Filter Type=Local Contrast; Intensiy Manual Threshold=4; Quality Manual Threshold=0.090; Filter objects between 120 and 10,000 Voxels). We extracted the area, as well as the fluorescence intensity in the VHH_{NP-1}-AF647 channel. Cell areas were scored as VHH+ if the mean intensity in the VHH_{NP-1}-AF647 channel was >80. The fraction of VHH+ cell areas of the entire cell area was calculated and plotted. SYTOX green-positive nuclei were detected with the spot detection tool of Imaris (Estimated Diameter=8 μm, Quality>10). Cells with distinct morphological features or fluorescence distribution were manually counted with the help of the counter function of Imaris. To quantify plasma membrane (PM) distribution of fluorescent GSDMD_{NT} fusions, fluorescence intensity profiles along a line cutting the cell were analyzed for each cell. If the fluorescence only co-localized with the plasma membrane marker, localization was categorized as ‘PM’, when fluorescence was only found in the cytosol and dropped at the plasma membrane, localization was counted as ‘cytosol’, and when fluorescence above background was found in the cytosol, but fluorescence still increased at the PM, localization was scored as ‘cytosol+PM’. When distribution of GSDMD-mNG_ins was quantified, ‘clear plasma membrane localization’ indicates that upon analysis of intensity profiles, the cell A) exhibits a distinct plasma membrane signal (clear peak of fluorescence at the rim of the cell, i.e. fluorescence appears as a relatively thin line), which requires that the focal plane cuts through the body of the cell and is not at the bottom or top of the cell; and that B) GSDMD_{NT}-mNG showed an equally crisp peak of fluorescence co-localizing with the plasma membrane staining (as apparent in intensity plot; GSDMD_{NT}-mNG staining appears as a thin line). This distinction was mostly used to exclude false positives, i.e. cells whose top or bottom were cut by the focal plane (no clear ring-like signal of plasma membrane marker), or cells with a large nucleus and very little cytoplasm next to it in the focal plane. In the latter case, the staining of GSDM_{NT}-mNG sometimes also appeared ring-like, although the fluorescence was observed in a broader rim with a less sharp increase and no co-localization with the plasma membrane. Of note, the seemingly low fraction of responding cells can be explained by the lower number of cells with A) membrane staining perpendicular to the focal plane, and B) sufficient GSDMD-mNG or endogenous GSDMD expression and cleavage. SYTOX green intensity in Incucyte experiments was analyzed with the Incucyte software to extract the integrated fluorescence per field of view. To quantify SYTOX green intensity per nucleus, nuclei were detected with CellProfiler using the ‘IdentifyPrimaryObjects’ function (default settings, minimal radius=10 pixel units, maximal radius=40 pixel units) and the mean fluorescence intensity extracted with the ‘MeasureObjectIntensity’ function. To quantify localization of VHH_{ASC}-AF647 to C1C-EGFP specks or nuclei from Z stacks (5 slices, 2 μm apart), we first detected both structures using the Imaris spot detection routine (Specks: C1C-EGFP channel, Estimated Diameter=2 μm, quality >10; nuclei: Hoechst channel, Estimated Diameter=8 μm, quality >1) and enumerated C1C-EGFP specks with a VHH_{ASC}-AF647 intensity in the center >65 as well as nuclei with mean VHH_{ASC}-AF647 intensity >100. The fraction of VHH_{ASC}-AF647-positive nuclei was corrected by the fraction of positive cells observed in cells stimulated in the presence of VHH_{NP-1} and VX (to correct variable background of cells that were negative for C1C-EGFP, but PI-/VHH-positive before treatment).

### Example 2—Identification of GSDMD-Specific Single-Domain Antibodies I

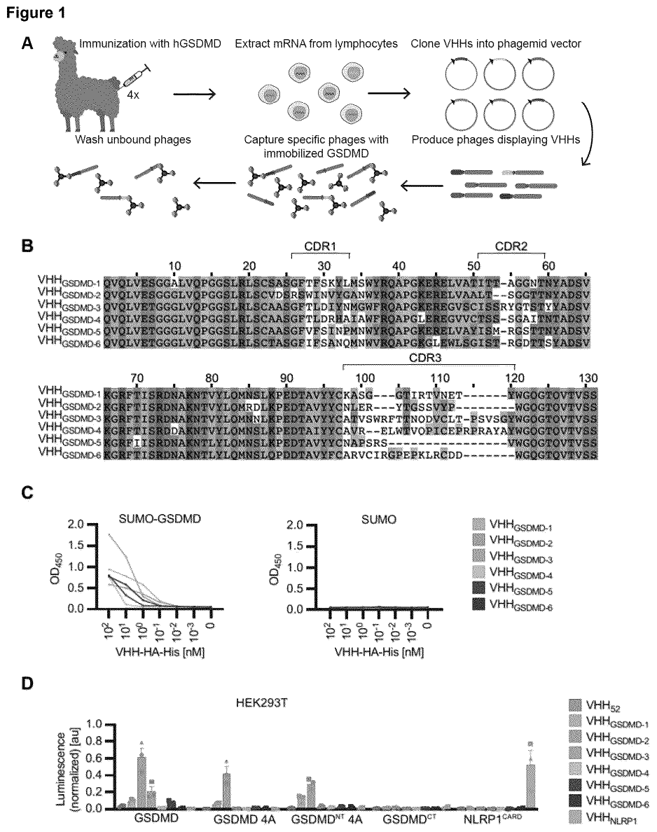

Specific inhibitors of GSDMD are scarce and apart from complete knockouts and overexpressed point mutants, no tools were available to perturb GSDMD to study pore formation in molecular detail in living cells. To overcome these shortcomings, single-domain antibodies (herein also referred to as nanobodies or VHHs) were raised against human GSDMD protein. An alpaca (Vicugna pacos) was immunized with bacterially expressed recombinant full length GSDMD protein. Phage display was employed to positively select GSDMD-specific VHHs, yielding 6 hits that substantially differ in their complementarity determining regions (CDRs) (FIG. 1A,B). ELISA experiments with decreasing concentrations confirmed the specificity against GSDMD (FIG. 1C). To test the functionality of the 6 GSDMD-specific nanobodies in the cytosol of living cells, LUMIER assays were performed: HEK 293T cells were co-transfected with expression vectors for HA-tagged nanobodies (VHH-HA) and fusions of different variants of GSDMD to Renillla luciferase (GSDMD-Renilla). HA-tagged nanobodies were subsequently immunoprecipitated from lysates. If the nanobody binds to GSDMD in the cells, GSDMD and luciferase activity is co-immunoprecipitated, resulting in a luminescent signal after addition of the Renilla substrate coelenterazine. This confirmed robust GSDMD binding of VHH_{GSDMD-2} and VHH_{GSDMD-3}, and some binding of VHH_{GSDMD-1} and VHH_{GSDMD-5} in the cytosol (FIG. 1D). To determine the domain of GSDMD bound by the nanobody, fusions of GSDMD^{NT} and GSDMD^{CT} to Renilla were also included. As overexpression of GSDMD^{NT} alone kills cells by pyroptosis, full length and the N-terminal domain of GSDMD mutant 4A, which no longer binds to membranes and does not cause pyroptosis were used. VHH_{GSDMD-1} and VHH_{GSDMD-2} clearly bind the N-terminal domain of GSDMD, while binding of VHH_{GSDMD-3} and VHH_{GSDMD-5} was affected by the 4A mutation and no clear conclusion was possible.

### Example 3—VHH_{GSDMD-1} and VHH_{GSDMD-2} Abrogate Pyroptosis

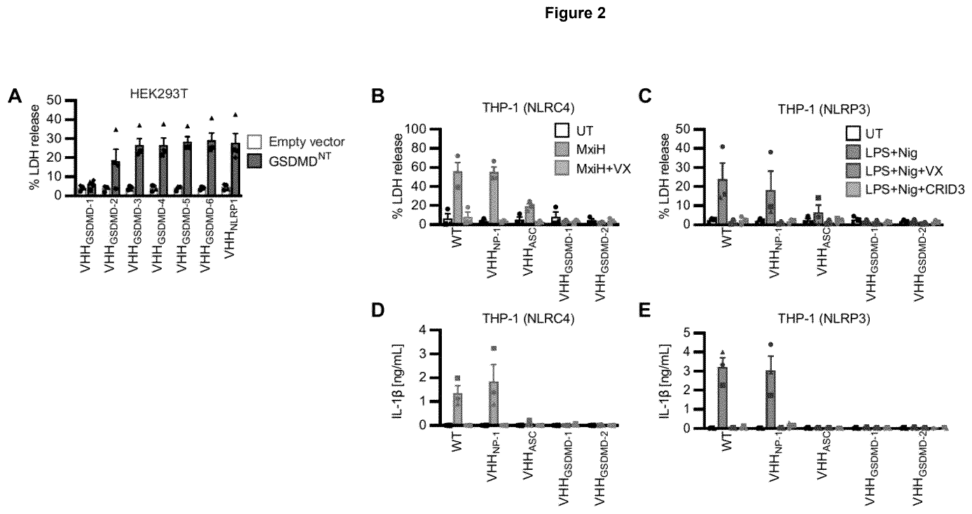

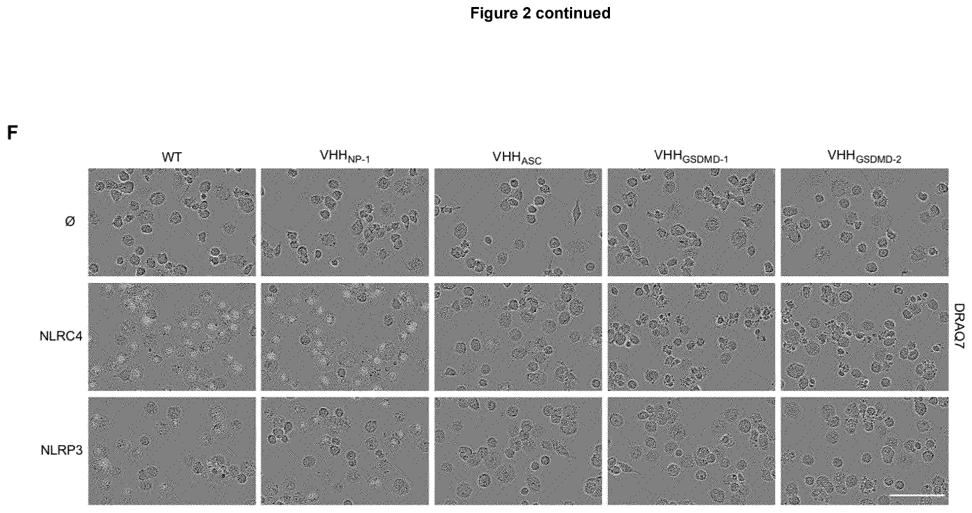

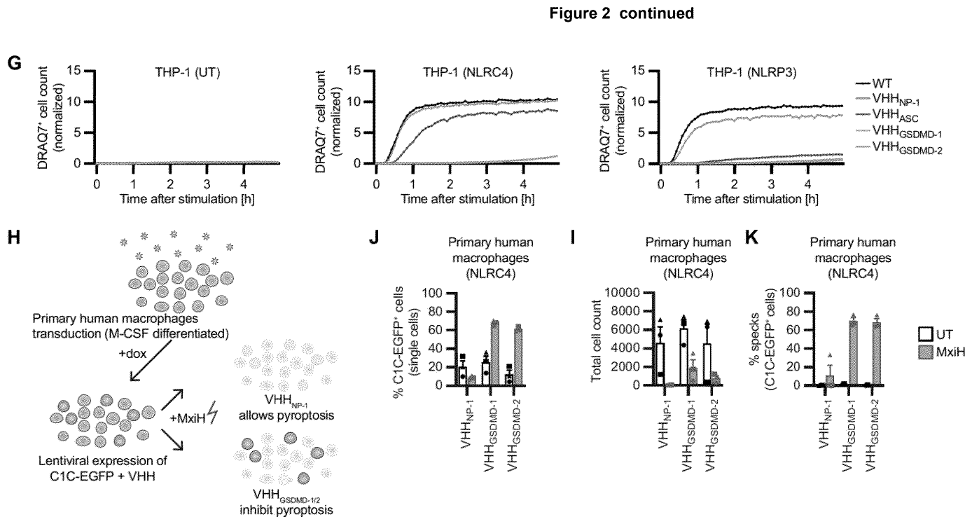

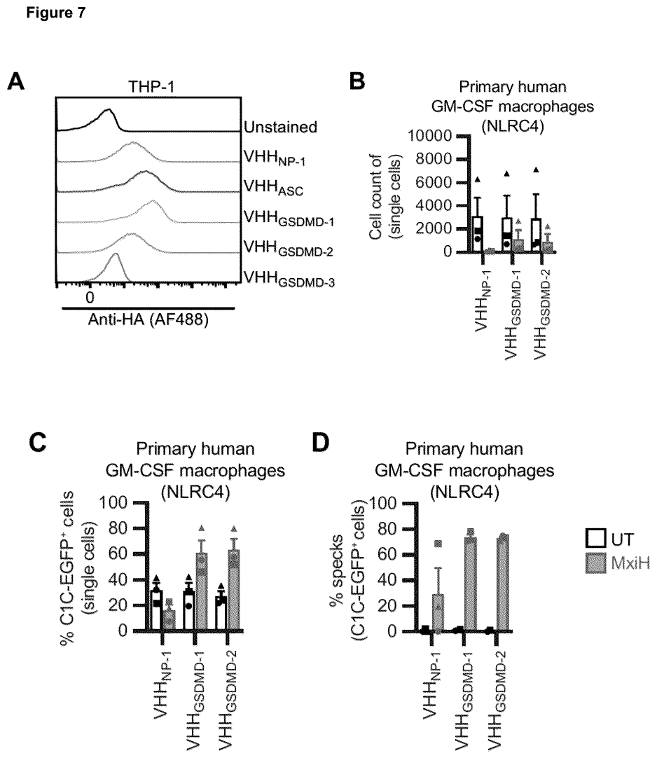

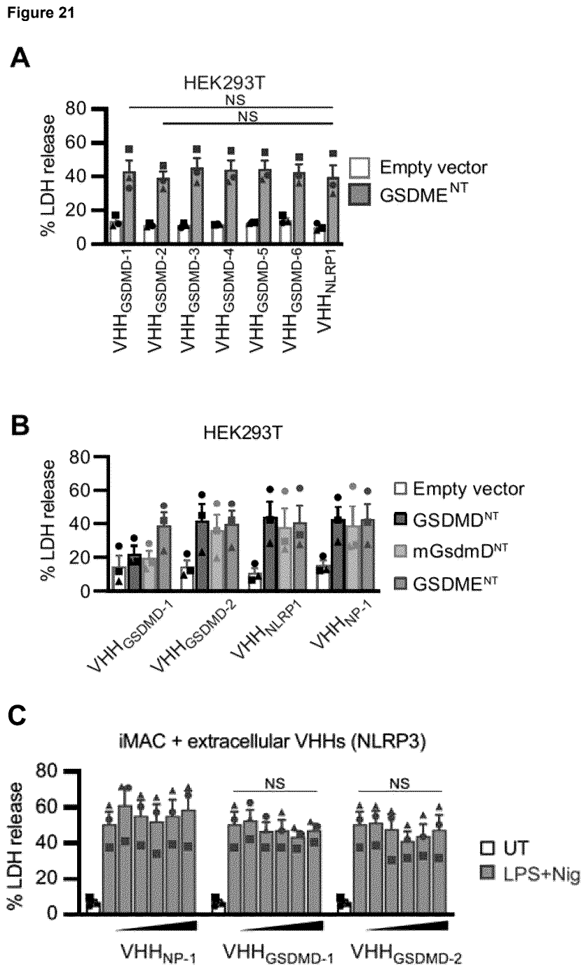

Next, it was investigated whether the identified nanobodies perturb GSDMD function if expressed intracellularly. To test the effect of the nanobodies on GSDMD^{NT}-induced LDH release, HEK 293T cells were co-transfected with expression vectors for GSDMD^{NT} and the indicated GSDMD nanobodies. Interestingly, VHH_{GSDMD-1} and to some extent VHH_{GSDMD-2} inhibited the release of LDH in HEK293T cells overexpressing GSDMD^{NT}, but not GSDME^{NT} (FIG. 21A), while control nanobodies and the other GSDMD nanobodies did not affect cell death, suggesting that VHH_{GSDMD-1} and VHH_{GSDMD-2} may inhibit pyroptosis (FIG. 2A). In order to validate this in a more relevant cell type, human myeloid THP-1 cell lines expressing the HA-tagged nanobodies in a constitutive manner under the strong EF1α promoter were generated. Since VHH_{GSDMD-3} did not express well in these cell lines, it was excluded from further analyses (FIG. 7A). THP-1 WT cells and cells expressing a unrelated nanobody against the nucleoprotein of influenza A virus (VHH_{NP-1}) were used as negative control, whereas VHH_{ASC} served as positive control, since it interferes with inflammasome formation and IL-1β release by impairing ASC^{CARD} interactions. The THP-1 cells were PMA-differentiated into macrophages and activated with either the Shigella needle protein MxiH, delivered with the anthrax toxin delivery system to induce NLRC4-inflammasome activation, or with LPS and nigericin to activate the NLRP3 inflammasome. Robust LDH and IL-1β release was observed in the control cell lines and it was confirmed that responses to both triggers completely depend on caspase-1, as they were abrogated by the caspase-1 inhibitor VX-765 (VX). Responses to LPS and nigericin treatment were mediated by NLRP3, as indicated by the sensitivity to NLRP3 inhibitor CRID3 (FIG. 2B-E). Strikingly, both VHH_{GSDMD-1} and VHH_{GSDMD-2} completely shut down the release of LDH (FIG. 2B,C) and IL-1 (FIG. 2D,E) to background levels after NLRC4 as well as NLRP3 inflammasome activation. Next, inflammasomes were activated and microscopically followed and the uptake of DRAQ7, a membrane-impermeable far-red fluorescent DNA dye, was quantified to monitor permeability of the plasma membrane over time. While DRAQ7 uptake was detected in almost all control cells within 1 h after treatment, no DRAQ7 uptake was apparent in the VHH_{GSDMD-1} and VHH_{GSDMD-2} expressing THP-1 macrophages (FIG. 2F,G). In line with these findings, cells expressing antagonistic nanobodies do not show any features of pyroptotic cell death, as opposed to the negative controls (FIG. 2F). It was concluded that cytosolic expression of VHH_{GSDMD-1} and VHH_{GSDMD-2} completely abrogates GSDMD-mediated effector functions.

Next, the functionality of the inhibitory GSDMD nanobodies in primary human M-CSF- and GM-CSF differentiated macrophages was also investigated. For this purpose, the primary human macrophages were transduced with lentivirus encoding the nanobody of interest in addition to our previously described fluorescent inflammasome reporter caspase-1^{CARD}-EGFP (C1C-EGFP), which is recruited to ASC specks via the caspase recruitment domain (CARD) of caspase-1 (FIG. 2H). To overcome restriction by SAMHD1 in macrophages, lentivirus was produced in cells expressing a fusion protein of SIVmac251 Vpx and HIV-1 NL4.3 Vpr. Vpx-Vpr is packaged into lentivirus particles as Vpr binds to the structural protein Gag, and thus delivers Vpx into target cells, which mediates the Cullin-4a-mediated proteasomal degradation of SAMHD1. This led to a transduction efficiency of around 10-30%, as measured by flow cytometry (FIG. 2I, FIG. 7C). Upon treatment with NLRC4-activating trigger MxiH, we observed a strong reduction in cell counts, as pyroptotic cells are too fragile to survive processing for flow cytometry (FIG. 2I, FIG. 7B). At the same time, it was observed that macrophages expressing antagonistic VHH_{GSDMD-1} or VHH_{GSDMD-2} and C1C-EGFP preferentially survived, as EGFP-positive cells now comprise 60-70% of the single cell population (FIG. 2J, FIG. 7C), while cells expressing control nanobody VHH_{NP-1} did not provide a survival benefit. This indicates that only the cells that expressed the antagonistic VHHs could survive the lethal trigger, and hence VHH_{GSDMD-1} and VHH_{GSDMD-2} prevent pyroptosis in primary human macrophages. To verify that MxiH treatment induced inflammasome assembly, recruitment of C1C-EGFP to ASC specks was detected by measuring width and height of the C1C-EGFP signal by flow cytometry. Robust inflammasome activation was observed in the majority of MxiH-treated cells that survived NLRC4 activation due to the expression of antagonistic GSDMD nanobodies, indicating that the surviving cells did respond to MxiH and survival was not due to any impairment of inflammasome assembly (FIG. 2K, FIG. 7D). Altogether, these results show a potent pyroptosis-inhibiting effect of VHH_{GSDMD-1} and VHH_{GSDMD-2} in both THP-1 macrophages and primary human macrophages upon NLRP3 and NLRC4 inflammasome activation.

### Example 4—Antagonistic Nanobodies Prevent the Oligomerization, but Still Allow Membrane Localization of GSDMD^{NT}

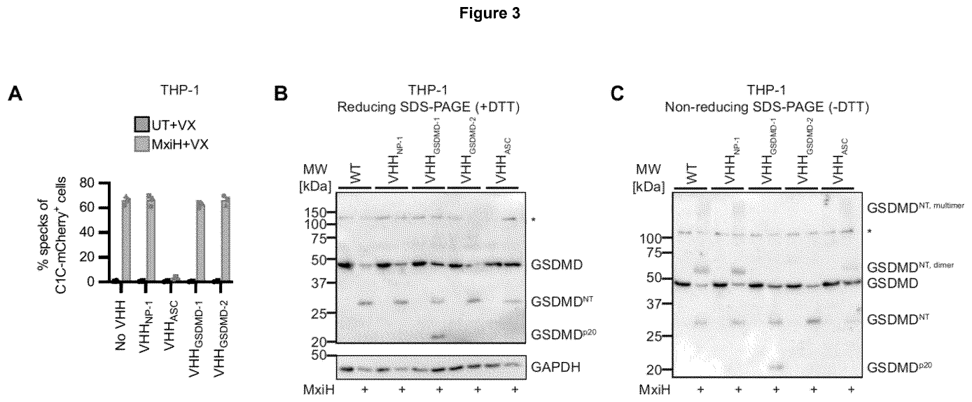

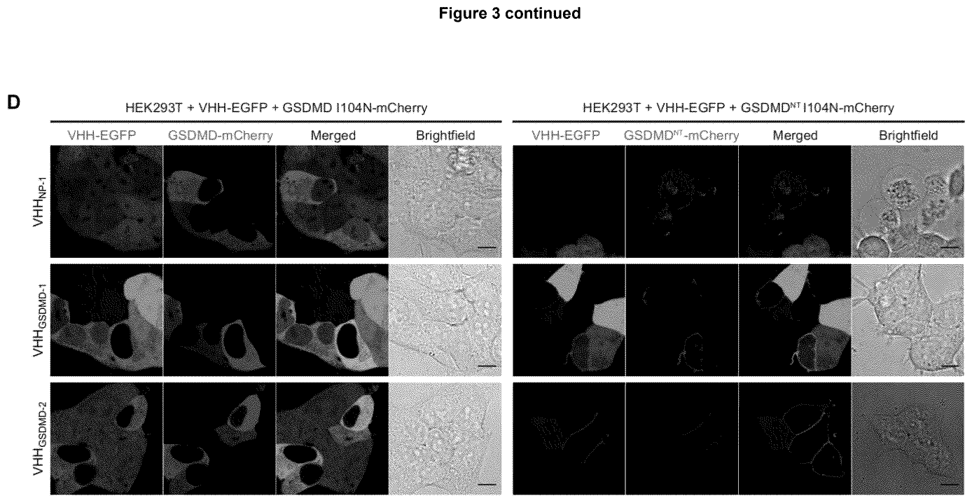

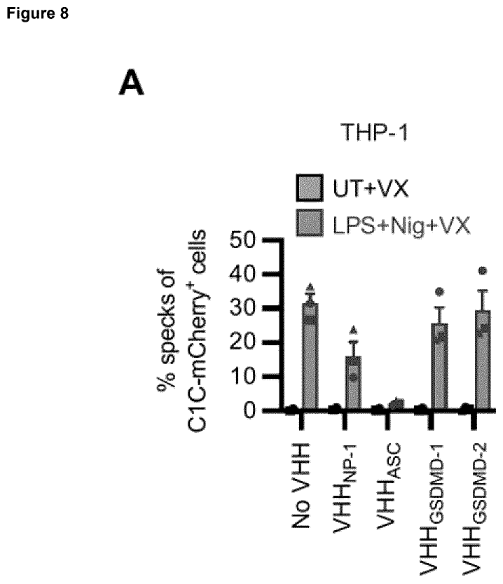

Next, it was attempted to find out what the mechanism of pyroptosis inhibition by the GSDMD nanobodies could be. To rule out any effect on the assembly of the inflammasome, the formation of ASC specks in THP-1 macrophages was analysed, which—in addition to the different VHH-EGFP fusions—also inducibly express the C1C-mCherry inflammasome reporter. Flow cytometry analysis upon MxiH delivery or stimulation with LPS and nigericin (in presence of VX to prevent pyroptosis and thus cell loss) proves that the nanobodies do not influence formation of ASC specks (FIG. 3A, FIG. 8A).

Next, it was analysed whether binding of VHH_{GSDMD-1} or VHH_{GSDMD-2} altered GSDMD levels or its processing by caspase-1. Lysates of MxiH-treated THP-1 macrophages were separated by SDS-PAGE under reducing conditions and analysed by immunoblot. No differences in GSDMD expression or cleavage to yield GSDMD^{NT} was detected (FIG. 3B).

It has previously been shown that GSDMD oligomers were revealed as high-molecular weight bands under non-reducing conditions. THP1 cell lysates were thus analysed after SDS-PAGE in the absence of DTT. In wt THP-1 cells and cells expressing control nanobodies, the formation of dimers and higher order oligomers in absence of DTT could be observed, which were no longer present in the lysates from VHH_{GSDMD-1} or VHH_{GSDMD-2} expressing cells (FIG. 3C). This demonstrates that the nanobodies interfere with the oligomerization of GSDMD^{NT}.

Since a system in which antagonistic nanobodies stabilized monomeric GSDMD^{NT} by preventing oligomerization had now been established, it was now possible to find out if the monomeric protein would be sufficient for membrane localization, or whether oligomerization would be a prerequisite. We therefore transfected HEK 293T cells stably expressing VHH-EGFP fusions with expression vectors for full length GSDMD-mCherry I104N or GSDMD^{NT}-mCherry I104N and followed localization by live cell confocal microscopy. The mutant I104N was reported to be less active and thus favors the detection of GSDMD pores before cells die from pyroptosis. The GSDMD-mCherry signal was evenly distributed in the cytosol irrespective of the nanobody expressed, confirming that full length GSDMD did not insert into the plasma membrane as expected (FIG. 3D). When GSDMD^{NT}-mCherry I104N was expressed in HEK 293T cells expressing control nanobody VHH_{NP-1}, it was observed that most cells appeared pyroptotic, while fluorescence was almost entirely found in internal structures or organelles in pyroptotic cells. This suggests that GSDMD^{NT} does not accumulate in the plasma membrane, indicating rapid removal, e.g. by membrane repair processes involving shedding of membrane vesicles. It cannot be ruled out that some GSDMD^{NT} is also recruited to other cellular membranes, as speculated earlier in the art. However, when GSDMD^{NT} was co-expressed in cells with VHH_{GSDMD-1} or VHH_{GSDMD-2}, GSDMD^{NT}-mCherry almost completely partitioned into the plasma membrane, where it co-localized with VHH-EGFP (FIG. 3D). The cells no longer showed signs of pyroptosis, in line with the expected inhibition of GSDMD pore formation. Similar results were obtained with wt GSDMD^{NT}-mCherry. Monomeric GSDMD^{NT} can thus insert into the plasma membrane, indicating that the required conformational changes for membrane integration occur in monomeric GSDMD after removal of the auto-inhibitory C-terminus. This strongly implies that GSDMD^{NT} only oligomerizes after inserting into the plasma membrane, and therefore most likely does not need to go through a prepore state comprised of oligomeric GSDMD^{NT} that is not inserted into the membrane. These observations allow two additional important conclusions: 1) GSDMD^{NT} is almost exclusively localized to the plasma membrane, rendering it less likely that insertion into other organelles such as mitochondria contributes to pyroptosis. 2) GSDMD^{NT}-mCherry is only observed in internal structures when GSDMD^{NT} is able to form pores, i.e. in cells with control nanobodies, suggesting that the observed structures result from the internalization of GSDMD pores and thus the result of endocytic repair mechanism that remove GSDMD pores.

We had unsuccessfully tried to visualize GSDMD pores in pyroptotic cells after inflammasome activation in THP-1 cells inducibly expressing GSDMD with mNeonGreen inserted after amino acid 270 (GSDMD-mNeonGreen_ins). Insertion of the fluorescent protein between the GSDMD^{NT} and the caspase-1 cleavage site yields GSDMD^{NT}-mNeonGreen after caspase-1 cleavage. Similar to our observations in HEK 293T cells expressing GSDMD^{NT}-mCherry, most of GSDMD^{NT}-mNeonGreen generated by caspase-1 cleavage localized to internal structures, but not to the plasma membranes. Given that VHH_{GSDMD-1} and VHH_{GSDMD-2} stabilized GSDMD^{NT} monomers in the plasma membrane in HEK 293T cells, we wondered if the antagonistic nanobodies also stabilized GSDMD^{NT}-mNeonGreen cleaved after inflammasome-mediated caspase-1 activation in relevant cell types. We thus generated THP-1 cells expressing VHH_{GSDMD-1}-HA or VHH_{GSDMD-2}-HA in addition to GSDMD-mNeonGreen_ins. When inflammasome activation was triggered in those cells, we observed GSDMD^{NT}-mNeonGreen in the plasma membrane of cells that did not exhibit morphological features of pyroptosis (FIG. 3E) This confirmed that GSDMD^{NT} released by cleavage of full length GSDMD also inserted into the plasma membrane as a monomer, before pores were formed by oligomerization. In conclusion, our inhibitory GSDMD nanobodies interfere with GSDMD^{NT} oligomerization, leaving cleavage and membrane localization in living cells intact with important implications for the mechanism of pore formation: We propose that monomeric GSDMD^{NT} is able directly insert into the plasma membrane and build up the pore monomer by monomer within target membranes without the need for prior oligomerization.

### Example 5—Inhibition of Pore Formation by Antagonistic GSDMD Nanobodies Augments Caspase-1 Activity and Triggers Caspase-1-Dependent Apoptosis

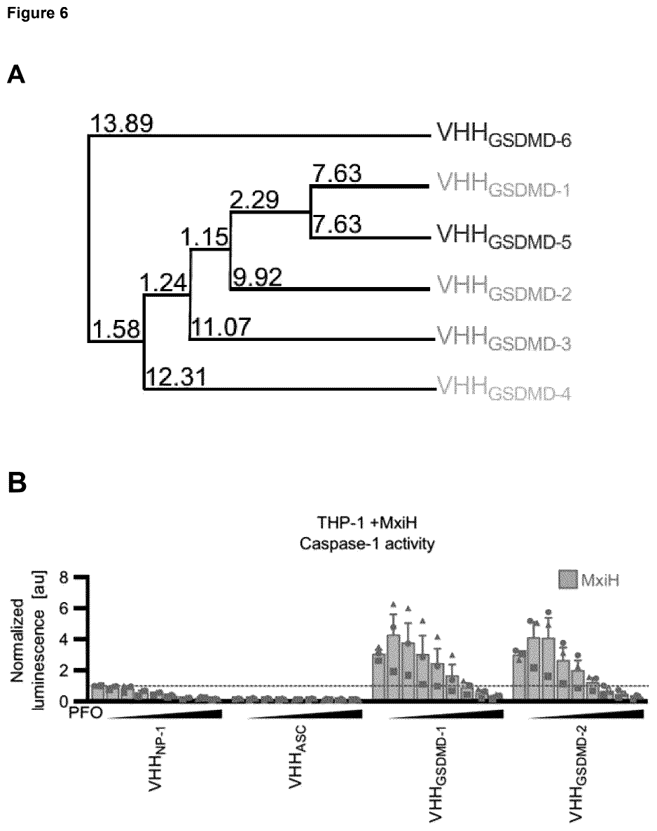

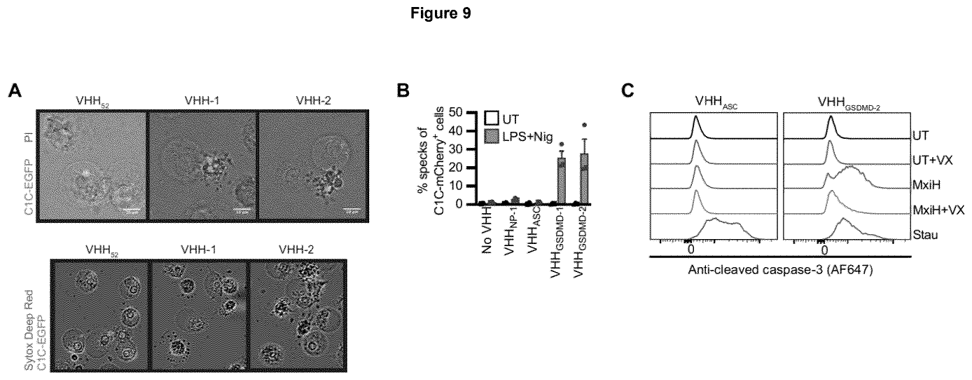

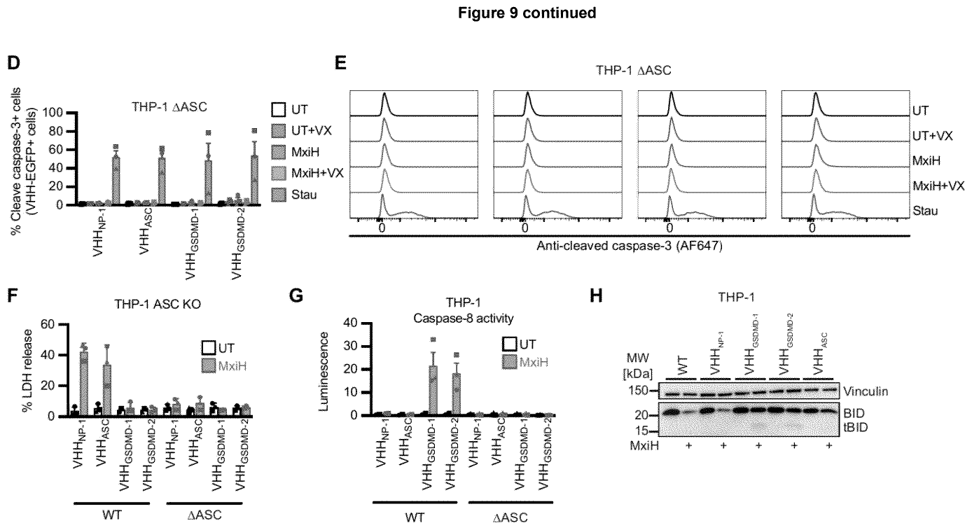

When analyzing THP-1 macrophages expressing different VHH-EGFP fusions in combination with the C1C-mCherry inflammasome reporter in more detail, we observed inflammasome assembly in presence of VHH_{GSDMD-1} and VHH_{GSDMD-2} as indicated by ASC speck formation 1 h post treatment (FIG. 4A). Interestingly, cells with ASC specks exhibited blebs or were fragmented into multiple vesicular fragments—morphologies more typically associated with apoptosis. Cells with ASC specks expressing the VHH_{NP-1} control, however, were round-up with a ‘balloon-like’ morphology as expected for cells undergoing pyroptosis (FIG. 4A). Similar apoptotic morphologies could be observed for the primary human macrophages transduced with antagonistic GSDMD nanobodies and C1C-EGFP as described above (FIG. 9A, FIG. 2H). Although we observed ASC specks in pyroptotic THP-1 cells expressing control nanobodies by microscopy, no specking cells were detected when treated cells were trypsinized and analyzed by flow cytometry (FIG. 4B, FIG. 9B). This confirms that pyroptotic cells were ruptured during processing. Of note, we typically inhibit caspase-1 with VX to allow quantification of inflammasome assembly by flow cytometry. In contrast, when cells expressing antagonistic GSDMD nanobodies were treated with inflammasome activators, ASC specks could be readily detected by flow cytometry in about 60% of the NLRC4-activated cells and 25% of the NLRP3-activated cells, indicating that ASC speck assembly in apoptotic cells could be analyzed flow cytometry (FIG. 4B, FIG. 9B). THP-1 macrophages expressing VHH_{ASC} do not assemble ASC specks as shown before in the art. This is another indication that the cells in presence of VHH_{GSDMD-1} or VHH_{GSDMD-2} do not succumb to pyroptosis, despite the activation of inflammasomes. To probe for bona fide apoptosis, we next stained the different PMA-differentiated THP-1 cell lines for cleaved caspase-3 upon MxiH treatment and quantified the fraction of cells positive for cleaved caspase-3 by flow cytometry. Staurosporine treatment was used as a positive control and resulted in more than 60% of the cells positive for cleaved caspase-3 (FIG. 4C). Both VHH_{GSDMD-1} and VHH_{GSDMD-2} expressing cells showed a clear population of cells positive for cleaved caspase-3, indicating that there is indeed activity of this apoptotic effector caspase (FIG. 4C,D, FIG. 9C). Interestingly, caspase-3 activation seems to be caspase-1-dependent since it was strongly reduced in presence of VX. Direct activation of caspase-8 by recruitment and autoproteolytic activation on ASC specks had been reported earlier in the art. As caspase-3 activation was largely blocked by VX, caspase-1 independent activation of caspase-8 does not seem to have a major contribution to caspase-3 activation. Yet the residual fraction of cells positive for caspase-3 cleavage after VX treatment may yield from direct activation of caspase-8 on ASC specks. In line with this interpretation, residual caspase-3 activation was no longer observed when cells expressed VHH_{ASC}, which prevents the formation of ASC specks. Flow cytometry analysis of caspase-3 cleavage in ΔASC THP-1 cells expressing the different VHHs also showed complete dependence on ASC speck formation (FIG. 9D,E). In theory, caspase-1 activation in NLRC4-stimulated inflammasomes could be ASC-independent due to a direct interaction of caspase-1^{CARD} and NLRC4^{CARD}, however, the lack of LDH release in ΔASC THP-1 macrophages make the ASC-independent activation of caspase-1 unlikely in our setup (FIG. 9F). Flow cytometry does not allow the measurement of pyroptotic cells and flow-cytometry-based analysis may thus fail to detect cleaved caspase-3 in the control cell lines that still undergo pyroptosis. To measure caspase-3 activity independent of cell death or rupture, we thus performed caspase Glo assays to measure the activity of caspase-3/7 in THP-1 macrophages upon MxiH treatment. Of note, caspase activity is determined in samples derived from the cells and the supernatant. Strong caspase-3/7 activity was observed in MxiH-treated cells expressing VHH_{GSDMD-1} and VHH_{GSDMD-2}, but not in cells undergoing expressing control nanobodies. Again, this activity was completely dependent on ASC, as no caspase-3/7 activity was observed in ASC knockout cells (FIG. 4D). Analysis of cell lysates by immunoblot confirmed cleavage of caspase-3, caspase-7, and the caspase-3 substrates PARP and GSDME only in those samples with caspase-3 activity, i.e. in cells in which antagonistic GSDMD nanobodies prevented pore formation (FIG. 4E). Remarkably, cleavage of GSDMDE in cells expressing antagonistic GSDMD nanobodies does not seem to be sufficient to assemble functional GSDME pores, as we did not observe pyroptosis, IL-1 release, or DRAQ7 uptake (FIG. 2C, E, F). GSDME thus does not play a major role in the death of VHH_{GSDMD}-expressing cells. Activation of caspase-3 also explains the presence of the GSDMD^{p20} band observed before (FIG. 3B), as addition of a caspase-3/7 inhibitor prevents the formation of this cleavage product (FIG. 4F). Of note, GSDMD^{p20} is only formed in cell lines expressing VHH_{GSDMD-1} but not VHH_{GSDMD-2}, which is possibly the result of differences VHHs masking different epitopes on GSDMD. Besides the presence of cleaved caspase-3 in VHH_{GSDMD-1} and VHH_{GSDMD-2} expressing THP-1 macrophages, we also detected processed caspase-8, processed caspase-9, and cleaved tBID in those samples with caspase-3 activity (FIG. 4G, FIG. 9G,H), indicating that both the intrinsic and extrinsic apoptosis pathway, or feedback mechanisms, may be involved. In theory, caspase-1, caspase-8, and caspase-9 can all catalyze the cleavage of caspase-3. However, we found that caspase-3 activity seems to be dependent on caspase-1 and ASC, suggesting that caspase-1 activated at the inflammasome seems to be the key regulator of the alternative cell death program. Coherently, we found that caspase-8 and caspase-9 activity are largely dependent on caspase-1 activity and inflammasome formation, as the cleavage of those caspases is strongly reduced in presence of VX and in ΔASC THP-1 macrophages (FIG. 4H, I). Only for caspase-8 there is some residual processing that can also be seen in the absence of GSDMD VHHs (FIG. 4H). This caspase-8 activation is, however, completely ASC dependent since it is absent in the THP-1 ΔASC cells (FIG. 4I), suggesting that a small portion of the caspase-8 is cleaved at the ASC speck, independent of caspase-1 as concluded previously in the art. We next quantified the caspase-1 activity of THP-1 macrophages upon MxiH stimulation using caspase-1 Glo assays. Surprisingly, we found that caspase-1 activity was increased up to a 6-fold in presence of VHH_{GSDMD-1} or VHH_{GSDMD-2} compared to the cells pyroptotic cells expressing VHH_{NP-1} (FIG. 4J). This is remarkable, as the assemble of ASC specks was comparable in all samples (FIG. 8A). We therefore hypothesize that the ability to form GSDMD pores has a profound impact on caspase-1 activity, suggesting GSDMD pores downregulate caspase-1 activity in a so far elusive mechanism. Caspase-1 ultimately serves as the master regulator for downstream cell death, as only the enhanced caspase-1 activity observed in the absence of GSDMD pores was sufficient to activate caspase-3 and apoptosis. One possible explanation for the relatively low caspase-1 activity in cells undergoing pyroptosis may be that ion fluxes through GSDMD pores and/or the dilution of cellular material into the supernatant compromises the stability or activity of caspase-1, even though the supernatant of the cells is included in the analysis. We thus treated THP-1 macrophages expressing VHH_{GSDMD-1} or VHH_{GSDMD-2} with MxiH in the presence of the pore-forming toxin perfringolysin O (PFO) from Clostridium perfringens, which forms pores of a diameter of 25-30 nm, i.e. of a similar if not slightly larger size than GSDMD pores. To avoid additional activation of NLRP3 by potassium efflux through PFO pores, NLRC4 stimulation experiments were performed in the presence of NLRP3 inhibitor CRID3. The augmentation of caspase-1 activity in the absence of GSDMD pores was reduced with increasing concentrations of PFO (FIG. 4L, FIG. 6B), suggesting that caspase-1 activity depends on changes in the cell that occur due to pore formation This indicates that some specific property of GSDMD pores impairs caspase-1 activity. In summary, we propose that the observed apoptosis in the absence of functional GSDMD pores is completely dependent on the inflammasome, with a central role for the augmented caspase-1 activity required for the processing of the initiator and effector caspases.

### Example 6—Recombinant Antagonistic GSDMD Nanobodies Inhibit Pyroptosis when Administered Extracellularly

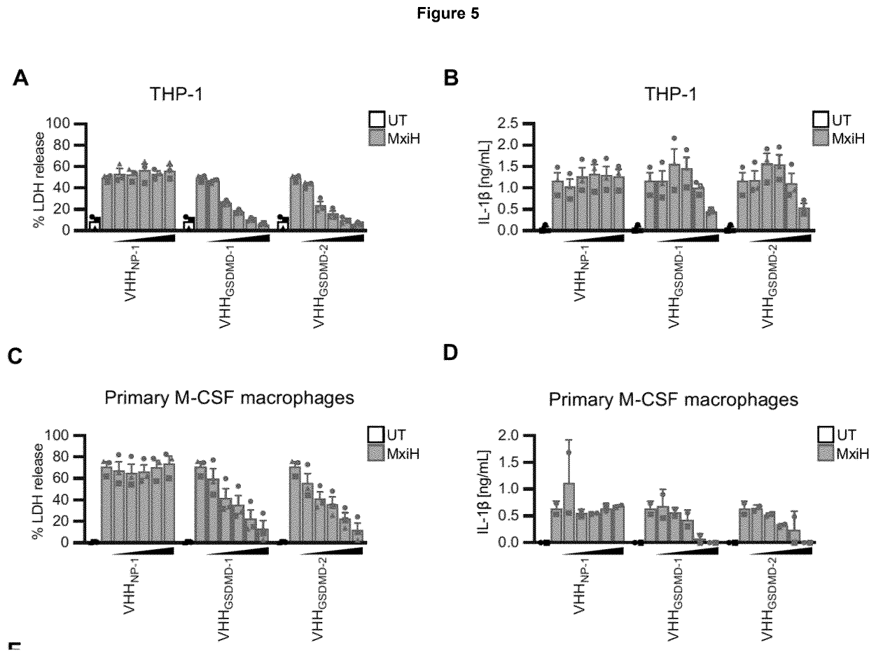

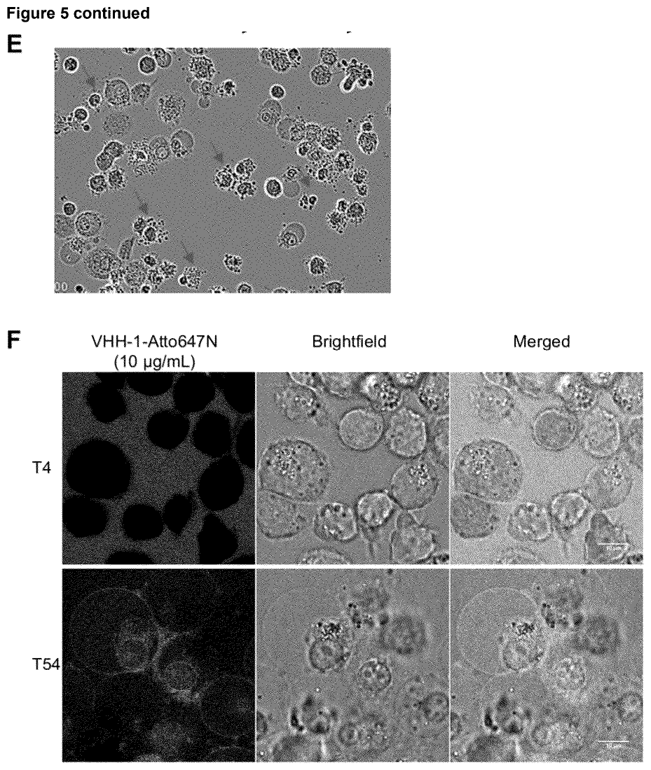

GSDMD is a highly sought-after drug target, although the development of specific GSDMD inhibitors was not successful to date. For any therapeutic application of the potent pyroptosis-inhibiting nanobodies we had discovered, delivery into the cytosol of target cells would be a prerequisite. Nanobodies are unable to cross intact cell membranes, but we had observed that fluorescent nanobodies can enter pyroptotic cells, likely passing through GSDMD pores. We thus speculate that early GSDMD pores would allow the passage of antagonistic GSDMD nanobodies administered from outside. We thus expressed the identified GSDMD nanobodies in bacteria and purified them. We next added increasing concentrations of the nanobodies to the culture medium of THP-1 macrophages treated with MxiH as an NLRC4 inflammasome activator. We observed a dose-dependent reduction in LDH release and importantly, higher concentrations of VHH_{GSDMD-1} and VHH_{GSDMD-2} reduced LDH release to background levels. In contrast, addition of increasing amounts of VHH_{NP-1} did not affect the release of LDH (FIG. 5A). The secretion of IL-1 was also substantially reduced, although the highest concentrations did not completely abrogate cytokine release (FIG. 5B). To validate these findings in a physiologically relevant in vitro model, we repeated the same experiments in primary human M-CSF macrophages (FIG. 5C,D, 29C). LDH release was inhibited in a dose-dependent manner and IL-1β secretion reduced to background levels at the highest concentrations. We hypothesize that nanobodies enter cells with inflammasomes upon formation of the first GSDMD pores, before the lytic stage of pyroptosis. Cytosolic nanobodies may thus prevent any further GSDMD pore assembly, which seems to be sufficient to allow cell survival. Based on our experiments with fluorescent GSDMD^{NT} fusions, it is likely that the early GSDMD pores are rapidly removed by membrane repair processes. Initial pore formation may well explain the remaining IL-1β secretion, since the cytokine may still be release through early sublytic GSDMD pores. Altogether, these results show that the nanobodies are potent inhibitors of inflammasome-induced pyroptosis when administered extracellularly and thus have interesting therapeutic potential. Importantly, early GSDMD pore formation may not be a terminal event, as cells could still be rescued by antagonistic GSDMD nanobodies.

### Example 7—Discussion of Results I

GSDMD pore formation is the effector mechanism that mediates cell death by pyroptosis as well as the non-conventional secretion of IL-1β and IL-18. The function of GSDMD was previously revealed in seminal loss-of-function screens and the structures of soluble full length GSDMD and GSDMD^{NT} pores was elucidated by structural biology. Yet critical molecular aspects of pore formation were unknown, as the process cannot be easily studied in relevant cells, primarily because pyroptotic cells do not weather sample preparation for microscopy and flow cytometry. In this invention, two GSDMD nanobodies, VHH_{GSDMD-1} and VHH_{GSDMD-2}, were established, which potently inhibit pyroptosis by preventing the oligomerization of GSDMD^{NT} and thus stabilize monomeric GSDMD^{NT}, rendering the steps of pore formation amenable to molecular study. Importantly monomeric GSDMD^{NT} was still able to insert into the plasma membrane and thus revealed that cleavage of GSDMD is sufficient to mediate the conformational changes necessary for membrane insertion. It was thus for the first time possible to observe this step in (live) human cells, supporting the conclusion that pores can grow monomer by monomer in a cell membrane. These results are in line with previous in vitro findings showing that in artificial membranes, smaller assemblies of human GSDMD^{NT} can assemble pores by transforming from arc- to slit- and finally to ring-shaped assemblies. Likewise, atomistic molecular dynamics simulations predicted that small GSDMD^{NT} assemblies can already form ion-conducting membrane pores and provide a plausible pathway to pore opening in intact biolayers. Earlier work had proposed the formation of GsdmA3 or GSDMD prepores composed of ring-like assemblies GsdmA^{NT} or GSDMD^{NT} associated with membranes in the globular conformation, resembling the conformation of the N-terminal domain in full length gasdermin. This model implied a coordinated conformational change in all subunits, giving rise to the eventual β-barrel structure that is inserted in the cell membrane. Our data suggests that monomers of GSDMD^{NT} undergo conformational changes that allow membrane insertion, even if oligomerization is prohibited with nanobodies, suggesting the assembly of prepores is not necessary for membrane insertion and therefore unlikely to be critical for pore formation. Recent atomic force microscopy data on GsdmA3 pores experimentally confirms membrane penetration of growing pores in different morphologies in vitro. While mobile prepore-like assemblies were observed to attach to membranes and disappear, none of them were observed to penetrate the membrane. Nevertheless, these findings do not completely rule out that two different pathways to pore-formation exist in parallel.

As the inhibitory nanobodies stabilize GSDMD into a monomeric intermediate, they can function as valuable tools to further elucidate molecular details of membrane insertion and pore formation in relevant cell types using live cell microscopy. Importantly, we found that fluorescent fusions of GSDMD^{NT} almost exclusively insert into the plasma membrane if oligomerization is inhibited. This demonstrates that the plasma membrane is indeed the primary target membrane of GSDMD^{NT} pores. By extension, similar antagonistic nanobodies may reveal to which membranes the N-termini of other gasdermin family members are targeted. Internal fluorescent structures containing fluorescent fusions of GSDMD^{NT} were only observed when pore formation was possible, i.e. in the absence of antagonistic nanobodies. Stabilization of monomers thus allows the distinction of the localization of GSDMD^{NT} before and after pore formation. Intracellular structures containing GSDMD^{NT} are thus a consequence of pore formation and very likely constitute GSDMD pores removed from the plasma membrane by endocytic membrane repair processes, suggesting that membrane shedding is not the only mechanism to dispose GSDMD pores, as described for MLKL. The inhibitory GSDMD nanobodies also provided new insights into the interconnectivity of the different cell death pathways in macrophages. Despite the presence of fully cleaved endogenous GSDMD in cells expressing VHH_{GSDMD-1} or VHH_{GSDMD-2}, macrophages undergo apoptosis that is dependent on inflammasome assembly, ASC specks, and caspase-1 activity, as previously reported for GSDMD KO cells and cells expressing catalytically inactive caspase-1. Importantly, we report for the first time that lack inflammasome activation in the absence of GSDMD pore formation strongly augmented caspase-1 activity. Only this enhanced activity resulted in efficient cleavage of caspase-3, caspase-7, and their substrates. We therefore propose a key regulatory role for the caspase-1 activity, which seems to be reduced when GSDMD pores form. Formation of PFO pores of similar size seems to also reduce caspase-1 activity as observed in presence of functional GSDMD pores, suggesting that a caspase-1 activity is altered by ion fluxes or any other direct or indirect consequence of pore formation. It is possible that the most active form of cleaved of caspase-1, the (p33/p10)₂ form, is stabilized by preventing or delaying the secondary cleavage between the CARD and p20, which is associated with loss of activity. GSDMD pore formation may also provide some elusive feedback signal to caspase-1 or the other caspases to dampen activity, e.g. altered ion concentrations or changes in the physical properties of the cytosol, e.g. in the reduction potential. Interestingly, apoptosis observed in our system followed a kinetic comparable to pyroptosis, with MxiH-stimulated cells already exhibiting morphologies of pyroptosis or apoptosis within 20-30 minutes after treatment, respectively. Remarkably, despite the efficient cleavage of GSDME in cells with assembled inflammasomes in the absence of GSDMD pores, no pyroptosis mediated by GSDME^{NT} was observed. This corroborates earlier findings suggesting GSDME-induced lytic cell death does not play a major role in macrophages. In contrast, overexpressed GSDME^{NT} in HEK 293T cells as well as GSDME cleaved by caspase-3 in keratinocytes are sufficient to initiate pyroptosis. This suggests that GSDME^{NT} may be subject to additional layers of regulation, as e.g. proposed for GSDMD^{NT}, which was proposed to require ROS for full activation. Since the number of diseases in which the inflammasome and GSDMD play a detrimental role keeps increasing, there is a growing interest in specific GSDMD inhibitors. Our proof of concept experiments with antagonistic nanobodies VHH_{GSDMD-1} and VHH_{GSDMD-2} therefore highlight some interesting therapeutic potential. At the crossroads of intracellular signaling upon pathogenic threats and DAMPs, targeting GSDMD would not only prevent inflammation upon canonical but also non-canonical inflammasome stimuli. We could show that the extracellular addition of the nanobodies drastically reduces pyroptosis and the release of the pro-inflammatory cytokine IL-1 in both PMA-differentiated THP-1 macrophages as well as M-CSF-differentiated primary human macrophages. We propose that the nanobodies enter the cells upon the formation of the first sublytic GSDMD pores, rendering further GSDMD^{NT} oligomerization and thus pore formation and pyroptosis impossible. One additional benefit of therapeutic application of recombinant nanobodies is that the nanobodies in this scenario only target cells that have already assembled GSDMD^{NT} pores and thus only gain access to cells relevant for the inflammatory response. The high specificity for both the target protein and the cellular state render antagonistic GSDMD nanobodies interesting for therapy. This is of particular interest, as other reported inhibitors are often cysteine-reactive compounds and thus lack specificity. The possibility to tailor nanobodies into bivalent or multivalent molecules may constitute a good basis for further optimizations. Lastly, nanobody-mediated survival of macrophages with cleaved GSDMD likely relies on membrane repair processes, which could be studied in more detail using this system.

In conclusion, it was shown by the present inventors that antagonistic GSDMD nanobodies afford unprecedented modes of intervention by stabilizing informative intermediates of GSDMD^{NT}. This functional perturbation not only allowed mechanistic insights into membrane insertion, pore formation, and removal of pores from the plasma membrane, but also provides an interesting proof of concept for the therapeutic application of recombinant nanobodies.

### Example 8—Identification of GSDMD Specific Single-Domain Antibodies II

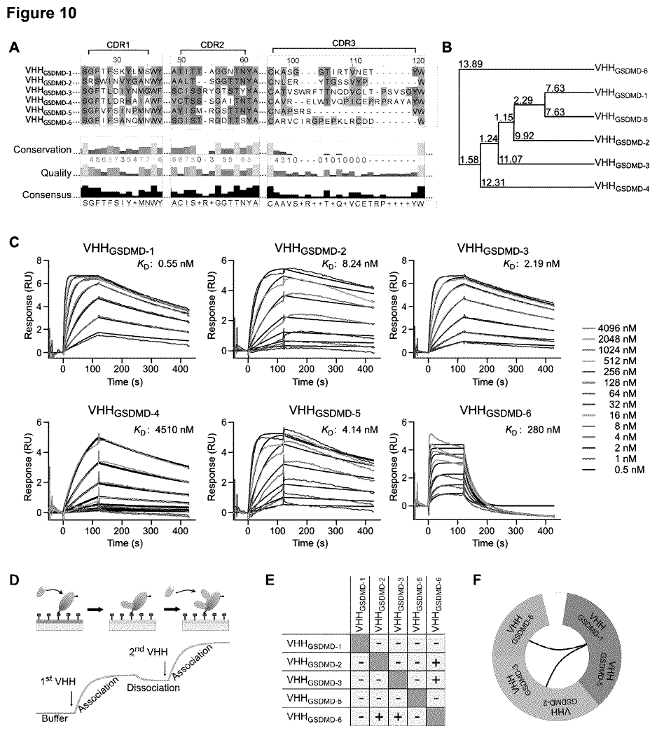

GSDMD targeting nanobodies were raised by immunization of an alpaca with full-length recombinant human GSDMD protein. Positive hits from serum were identified by an enzyme-linked immunosorbent assay (ELISA) described elsewhere. The potential binders differed by at least 7.6% in their amino acid sequence and showed great variety in the lengths and composition of their complementarity determining region 3 (CDR3) (FIG. 10A, B). The binding of the nanobodies was validated using surface plasmon resonance (SPR) spectroscopy and binding affinities were determined by applying multi-cycle kinetics (FIG. 10C). Nanobodies VHH-1, -2, -3, and -5 bound to GSDMD with high affinities in the nanomolar range and displayed rapid association and slow dissociation rates. The tightest binder was VHH-1 with a dissociation constant of 0.55 nM. VHH-2, -3 and -5 had dissociation constants of 8.24 nM, 2.19 nM, and 4.14 nM, respectively. In contrast, VHH-4 and VHH-6 exhibited significantly lower binding affinities in the micromolar range. Due to its low affinity, VHH-4 was excluded from further SPR experiments.

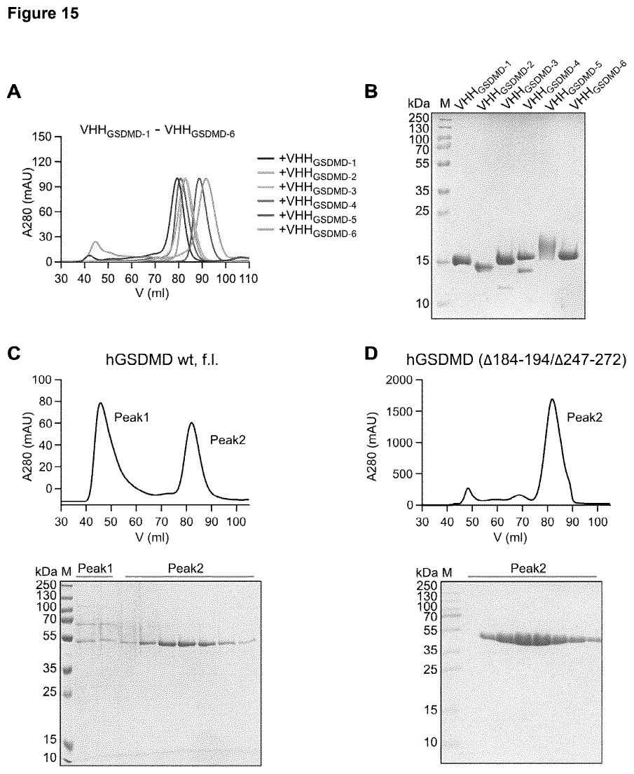

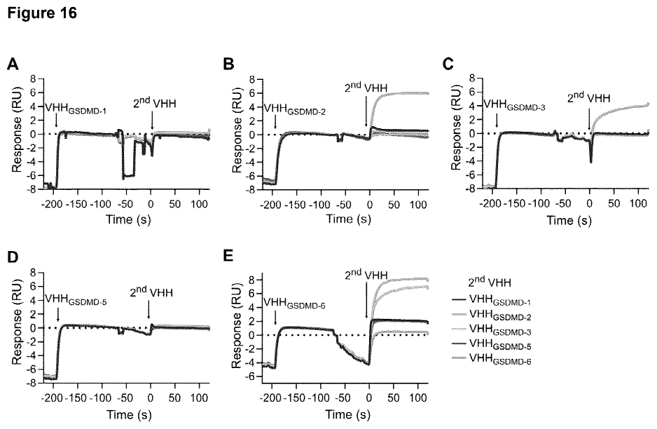

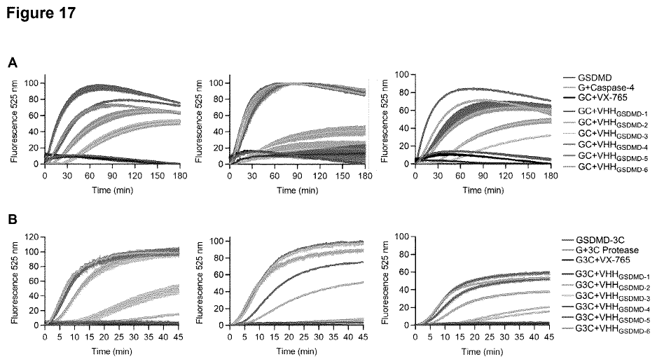

Binding epitopes of the nanobodies on GSDMD were analyzed using an SPR-based epitope binning assay (FIG. 10D-F, FIG. 15). Chemically biotinylated GSDMD was immobilized on an SPR sensor chip and nanobodies were injected as analytes in a pair-wise manner to test whether they compete for overlapping or distinct epitopes on GSDMD (FIG. 10D). VHH-1 and VHH-5 competed for an overlapping epitope with all other nanobodies (FIG. 15). VHH-2 and VHH-3 competed for one epitope but for both nanobodies, additional binding of VHH-6 was observed (FIG. 10E). According to these observations, VHH-1 and VHH-5, as well as VHH-2 and VHH-3, were grouped into one epitope bin, whereas VHH-6 stands alone (FIG. 10F).

### Example 9—Single-Domain Antibodies of the Present Invention Inhibit the Assembly of Functional GSDMD Pores In Vitro

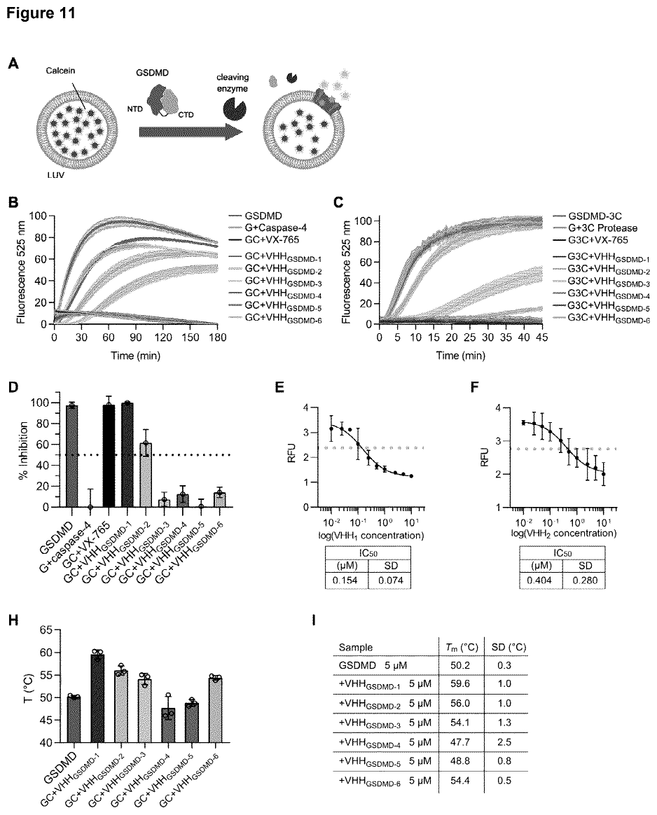

An in vitro liposome leakage assay was used to test whether nanobody binding affects the formation of functional GSDMD pores. GSDMD and nanobodies were added in equimolar ratios to calcein-packed liposomes and after the addition of caspase-4, calcein release through GSDMD pores was followed by measuring the fluorescence at 525 nm (FIG. 11A). As a control, we used the caspase inhibitor VX-765 which inhibited the calcein release completely (FIG. 11B). The addition of VHH-1 inhibited the calcein release to the same extent as the addition of VX-765, indicating that the assembly of functional GSDMD pores was fully abrogated. VHH-2 and VHH-3 also had an inhibitory effect, although to a lesser extent than VHH-1. The addition of VHH-6 slowed the calcein release down but the maximum fluorescence observed without the addition of nanobody was still reached. VHH-4 tended to increase the calcein leakage and VHH-5 had no remarkable effect on GSDMD pore formation (FIG. 11B). Comparable results were obtained when GSDMD-3C, a construct in which the caspase recognition site was replaced by a 3C protease recognition site, and 3C protease were used for the experiment. Also in this experiment, VHH-1, -2, and -3 inhibited the calcein release through GSDMD pores, whereas VHH-4, -5, and -6 did not inhibit GSDMD pore assembly (FIG. 11C).

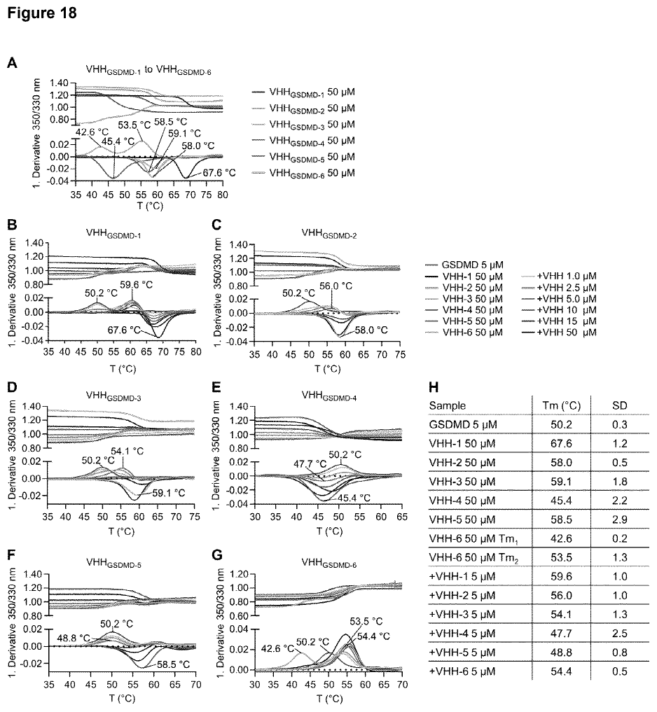

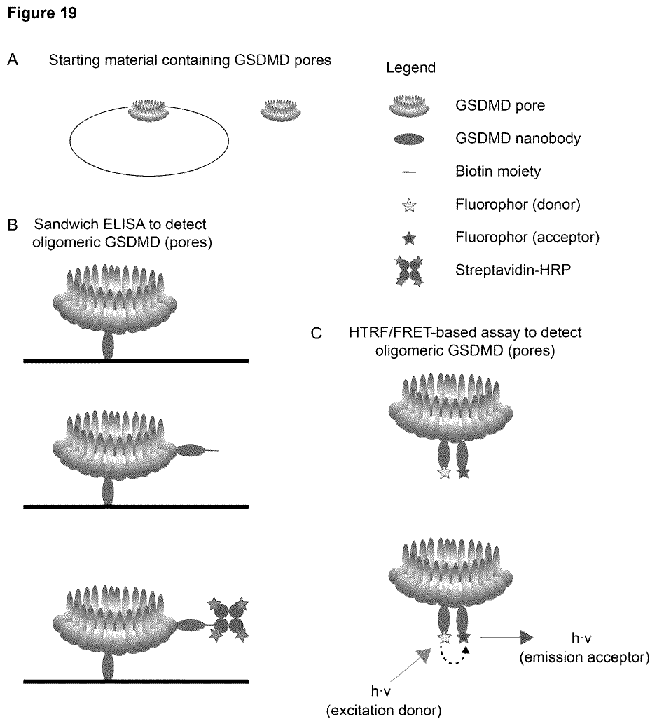

The thermal stability of the nanobodies and their impact on the thermostability of GSDMD was further analyzed using a thermal shift assay by nano-differential scanning fluorimetry (FIG. 11D, E and FIG. 18). The nanobodies were titrated to GSDMD in increasing concentrations, revealing a peak of fluorescence shift distinct from the peaks observed for GSDMD or nanobody alone, which indicated complex formation. At equimolar concentrations, the three inhibiting nanobodies (VHH-1, VHH-2, and VHH-3) increased the thermal stability of GSDMD by up to 9.4° C. In contrast, the non-inhibitory nanobodies VHH-4 and VHH-5 had a slightly destabilizing effect and decreased the melting temperature of GSDMD by up to 2.5° C. VHH-6 slowed down pore formation in the leakage assay and increased the thermal stability of GSDMD by 4.2° C.

### Example 10—Crystal Structure of GSDMD in Complex with Two Nanobodies

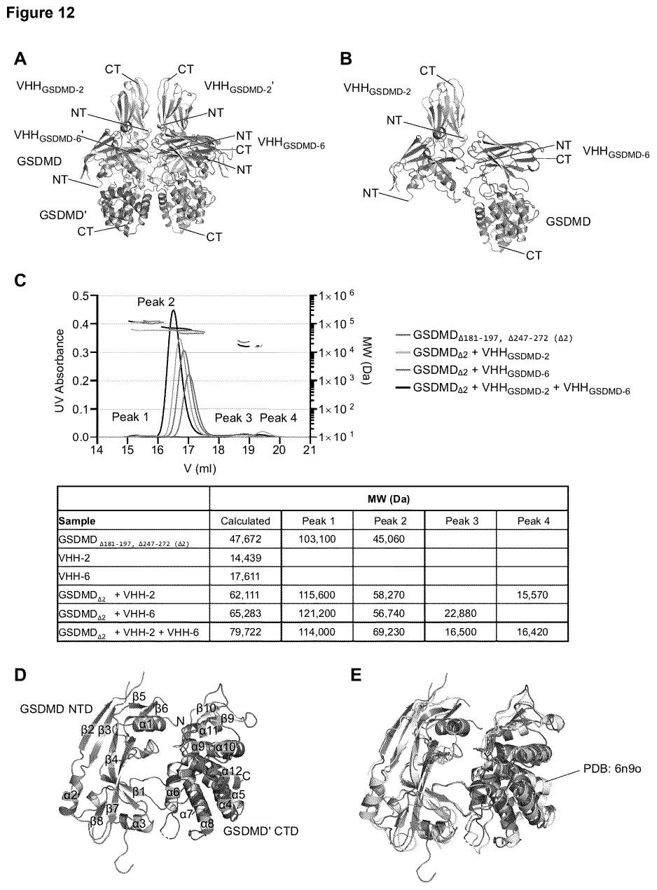

To map the epitopes of the nanobodies in detail and to shed light on the molecular mechanism by which VHH-1, -2, and -3 inhibit GSDMD pore formation, we initiated crystallization studies of the GSDMD-nanobody complexes. For this, we used a GSDMD construct where the linker region (residues 247-272) and residues 184-194 in the NTD were deleted to prevent precipitation during crystallization. Crystallization trials were successful for a tripartite complex consisting of GSDMD, VHH-2 and VHH-6, and well-diffracting crystals were reproducibly grown using this protein combination. We determined the crystal structure of the complex at 1.9 Å resolution by molecular replacement using the structures of human GSDMD (PDB 6N90) and a BC2 nanobody (PDB 5IVO) as search models. GSDMD and nanobodies are found in 1:1:1 stoichiometry with the two nanobodies unambiguously identified by their characteristic CDR regions. Two heterotrimeric GSDMD-VHH-2-VHH-6 complexes form the asymmetric unit of the crystal lattice and were refined to a R_{work} of 21.2% and R_{free} of 24.9% with excellent stereochemistry (FIG. 12A, Table 1).

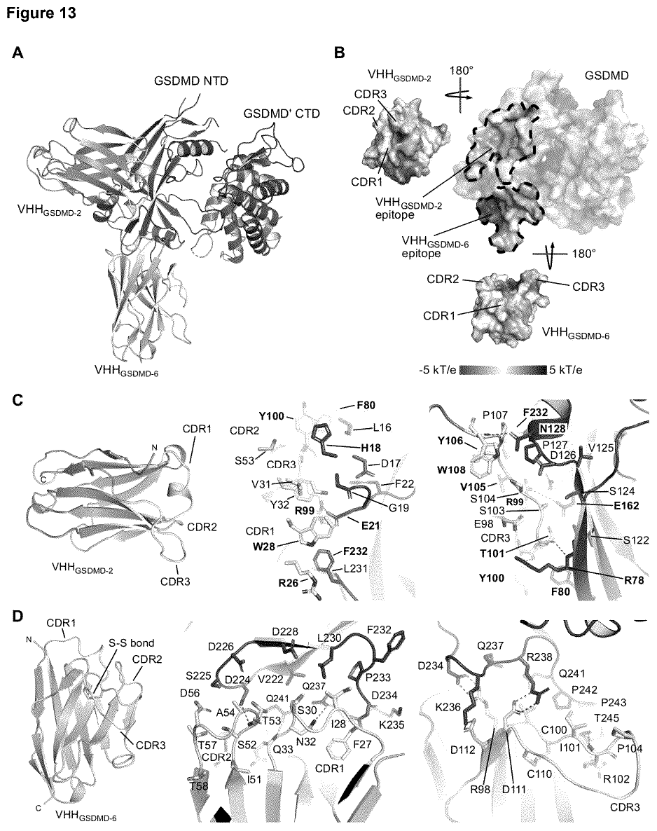

TABLE 1
Crystallographic data collection and refinement
statistics, related to FIGS. 12 and 13

GSDMD-VHH-2-VHH-6 complex

Data collection ^{a}

| Beam line | | DESY PETRA III p13 |
|---|---|---|
| Wavelength (Å) | | 0.976255 |
| Space group | | P 3₁ |
| Unit cell: | a, b, c (Å) | 108.35, 108.35, 124.04 |
| | α, β, γ (°) | 90, 90, 120 |

| | | |
|---|---|---|
| Resolution range (Å) | 93.84-1.86 | (1.926-1.86) |
| Unique reflections | 136,5353 | (13,351) |
| Multiplicity | 2.0 | (2.0) |
| Completeness (%) | 99.66 | (96.73) |
| Mean I/sigma(I) | 17.43 | (0.73) |
| Rₘₑₐₛ | 0.02305 | (1.209) |
| CC_{1/2} | 1.0 | (0.443) |
| Reflections used in | 136,351 | (13,167) |
| refinement | | |
| Reflections used for R-free | 1997 | (193) |

Refinement

| Model content | A, D: GSDMD (1-484, | |
|---|---|---|
| | D184-194, D247-272) | |
| | B, E: VHH-2 (1-118) | |
| | C, F: VHH-6 (1-123) | |
| # of atoms macromolecules | 9700 | |
| # of ligands | 0 | |
| # of solvent | 559 | |
| Solvent content (%) | 50 | |
| R_{work} | 0.2124 | (0.3297) |
| R_{free} | 0.2493 | (0.3204) |

| | |
|---|---|
| RMS deviations bonds [Å] | 0.008 |
| RMS deviations angles [°] | 0.79 |
| Ramachandran favored (%) | 96.24 |
| Ramachandran allowed (%) | 3.35 |
| Average B-factor | 49.55 |
| Macromolecules | 49.621 |
| ligands | — |
| solvent | 48.37 |
| PDB accession code | 7Z1X |

^{a} Values in parentheses are for the highest resolution shell.
R_{free}-value is equivalent to the R-value but is calculated for 5% of the reflections chosen at random and omitted from the refinement process.

The two GSDMD molecules found in the structure form a dimeric complex in which the NTD of one GSDMD molecule is tightly interacting with the CTD of the other, resulting in a buried surface area of 4018 Å² counting both molecules. Looking at a single heterotrimeric complex, VHH-2 is bound to the NTD of the GSDMD, whereas VHH-6 interacts with the NTD as well as the CTD and stabilizes the N- and CTD folding apart in the GSDMD molecule (FIG. 12B).

Since GSDMD has not been reported to form dimers before, we hypothesized that the complex formation observed might be an artifact of crystallization. Therefore, we performed SEC-MALS analysis and found that the complex of GSDMD, VHH-2, and VHH-6 displayed a molecular weight of 69.2 kDa, consistent with a 1:1:1 complex with calculated molecular weight of 79.7 kDa. For this reason, we conclude that the dimerization of the two heterotrimeric complexes occurred during crystallization. The interaction of the N- and C-terminal domains of the two GSDMD molecules resembles the interactions between both domains observed in the previous GSDMD structure (PDB 6N90) and superimposition of the complex with the previous structure results in a root mean square deviation of 2.311 Å over 2244 Ca atoms (FIG. 12D, E).

### Example 11—Binding Interfaces of GSDMD-VHH Interactions

The CDR1, -2 and -3 segments of VHH-2 comprise 10, 7 and 11 residues, respectively, and the nanobody backbone is stabilized by a conserved disulfide bond between C22 and C95. The interface of VHH-2 and GSDMD is mainly built by CDR1 and CDR3 of VHH-2, whereas CDR2 does not contribute to the interaction significantly. In contrast, for VHH-6 all three CDRs are involved in binding to GSDMD, and the nanobody backbone contacts GSDMD as well (FIG. 13A). Electrostatic interactions are crucial for the interaction of both nanobodies with GSDMD. The positively charged CDRs of VHH-2 bind to an acidic cleft on the GSDMD surface involving residues E21, D126, and E162. The CDRs of VHH-6 contact with the acidic residues D224, D226, D228, D234, and D275, and the VHH-6 backbone contacts residues E448 and E459 on the GSDMD surface (FIG. 13B). Binding of VHH-2 results in a buried surface area of 1521 Å² counting both molecules. A pronounced salt bridge is formed by Glu21 in the GSDMD NTD and Arg99 in CDR3 of VHH-2. Moreover, Arg78 on the GSDMD surface forms hydrogen bonds with Tyr100 and Thr101 in the CDR3 and an additional hydrogen bond can be found between the CDR3 residues Trp108 and Asn128 on the GSDMD surface.

Binding of VHH2 to GSDMD results in a buried surface area of 1521 Å² counting both molecules. A pronounced salt bridge is formed between R99 in CDR3 of GSDMD2 and E21 in the GSDMD N-terminal domain, complemented at the opposite side by a weak interaction to E162 of GSDMD. Moreover, the backbone carboxyl groups of neighbouring residues Y100 and T101 in the CDR3 form intermolecular hydrogen bonds with R78 of GSDMD. Another hydrogen bond is formed between the CDR3 residue W108 and N128 on the GSDMD surface. Additional hydrophobic contacts are formed between F232 of GSDMD with V105, Y106, R26 and W28 of GSDMD2 as well as Y100 in the CDR3 of GSDMD2, which is sandwiched between H18 and F80 of GSDMD (FIG. 13C).

VHH-6 was indispensable for the crystallization of high-resolution GSDMD-nanobody complexes by serving as a crystallization chaperone. The CDR3 of VHH-6 is particularly long, comprises 15 residues, and is stabilized by an additional disulfide bond between Cys100 and Cys110, which contributes to its indistinguishable identification (FIG. 13D). Various interactions can be found between the CDRs 1 and 2 and the NTD of GSDMD. Residues Asn32 and Gln33 in the CDR1 form hydrogen bonds with Gln237 and Gln241, whereas CDR2 residue Thr53 contacts Asp224 on the GSDMD NTD (FIG. 13D). The CDR3 of VHH-6 forms several hydrogen bonds and salt bridges with the NTD of GSDMD. Prominent salt bridges are found between residues Arg98 and Asp11 in the CDR3 of VHH-6 and residues Asp234 and Arg238 on GSDMD, respectively. Several residues in the VHH-6 backbone comprising residues 39, 42-45, 47, 95, and 112-115 as well as residues 104-112 in the CDR3 contact the CTD of GSDMD which might contribute to the role of VHH-6 in facilitating crystallization.

### Example 12—Pyroptosis is Inhibited by Blocking Oligomerization of the GSDMD NTD

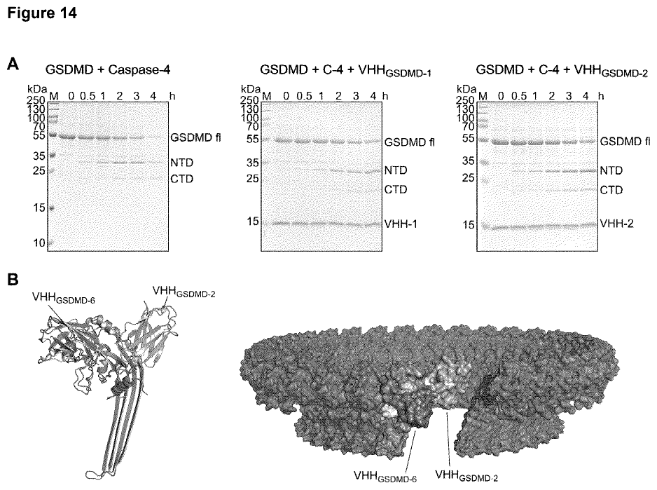

We have shown that VHH-1, VHH-2 and VHH-3 inhibited the assembly of functional GSDMD pores in vitro, leading to the question, by which mechanism pore formation is abrogated. Both nanobodies were found to bind to an overlapping epitope on the GSDMD NTD in the SPR-based epitope binning experiment and both nanobodies did not affect GSDMD cleavage by caspase-4 as observed on SDS-PAGE (FIG. 14A). We superimposed our structure of the nanobody bound GSDMD NTD with the cryo-EM structure of the activated GSDMD NTD (PDB 6VFE) previously determined by the Wu lab. The superimposition shows that VHH-2 and VHH-6 bind to the globular part of the activated NTD. Whereas VHH-6 binds on top of the globular rim of the GSDMD pore and does not interfere with oligomerization, VHH-2 binds in the oligomerization interface of the single N-termini and therefore sterically inhibits pore assembly (FIG. 14B). If blockage of GSDMD pore assembly also affects membrane binding and insertion remains to be investigated.

### Example 13—Discussion of Results II

GSDMD is the enforcer of pyroptosis, mediating the final common step of all inflammasome pathways. Since pyroptosis is implicated in many diseases, a deep understanding of the mechanisms underlying GSDMD pore formation and regulatory processes is essential. Inhibiting GSDMD is an attractive strategy to treat excessive inflammation and requires GSDMD-specific interacting molecules. To date, three small molecule inhibitors (necrosulfonamide, disulfiram and dimethyl fumarate) are known that covalently modify Cys191 in the GSDMD NTD and effectively prevent pyroptosis in cells and suppress inflammatory responses in murine models. However, this class of inhibitors has a serious disadvantage: due to their cysteine reactivity the compounds are not specific to GSDMD and binding to off-targets might cause unwanted side-effects in the human body. In this study, we characterized six unique GSDMD targeting nanobodies that bind with varying affinities to the NTD of GSDMD. Three of these nanobodies (VHH-1, -2, and -3) inhibited GSDMD pore formation in an in vitro liposome leakage assay. The high-resolution crystal structure of GSDMD in complex with one inhibitory and one non-inhibitory nanobody revealed, that the inhibitory nanobody sterically blocks the assembly of the GSDMD pore by binding to an epitope residing in the oligomerization interface of the activated GSDMD NTD. While pore formation is inhibited, caspase cleavage is not affected.

By sterically inhibiting oligomerization instead of targeting reactive cysteine residues in GSDMD, our nanobodies provide a new mechanism of pyroptosis inhibition and cell assays as well as in vivo studies will be required to investigate the full potential of the nanobodies as GSDMD specific drugs. GSDMD is an intracellular protein and the delivery of antibodies and nanobodies to the cytoplasm has long been limited due to their inability to cross the plasma membrane. Nevertheless, recent studies showed that nanobodies can be delivered to the cytoplasm of cells using cell penetrating peptide fusions and nanobody mRNA could be delivered using gene therapy approaches. Our SPR based binding experiments were performed in the presence of 5 mM DTT, indicating that the nanobodies remain their binding ability also under reducing conditions and should not be affected by the reducing milieu of the cytoplasm. Instead of being applied as drugs themselves, our nanobodies could also facilitate the development and characterization of new GSDMD specific small molecule inhibitors, due their function as crystallization chaperones, which enabled the generation of fast growing, reproducible, well diffracting GSDMD crystals for X-ray crystallography and the determination of high-resolution structures.

Apart from that, nanobodies are versatile tools and find broad applications in many research areas. For instance, they can be fluorescently labeled to visualize their target protein for fluorescence microscopy approaches or engineered to relocate their antigen to a specific cellular compartment. Taken together, our non-inhibitory and inhibitory nanobodies are useful tools for studying GSDMD biology by using them as specific inhibitors, fluorescent labels in imaging approaches, or crystallization chaperones for high-resolution GSDMD structures and can be used as templates for the development of nanobody-based GSDMD inhibiting drugs.

### Example 14—Further Characterization of the Single-Domain Antibodies with Regard to their Specificity, and the Mechanism of Pore Formation

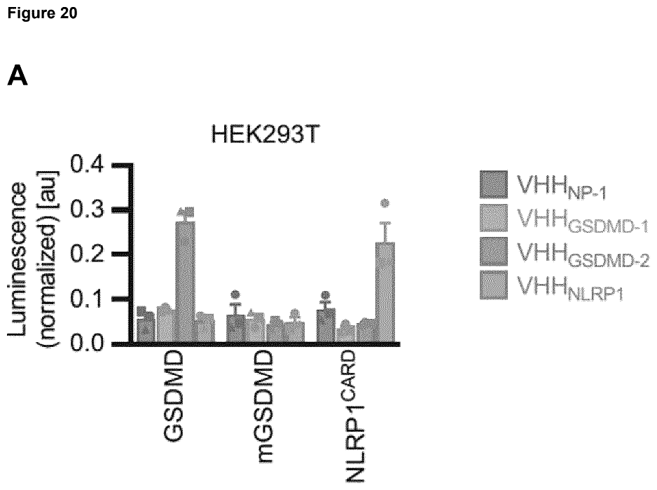

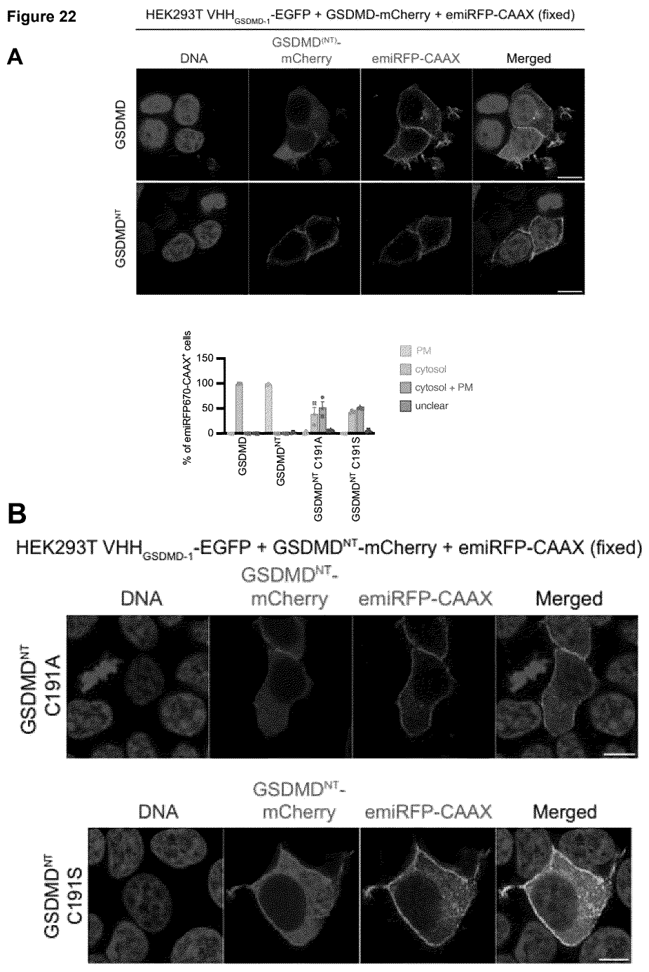

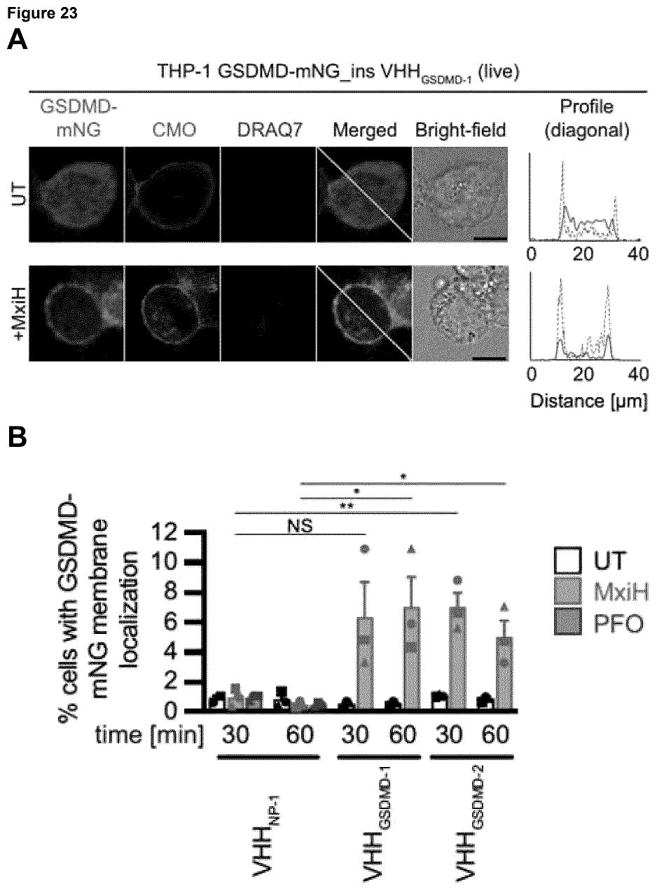

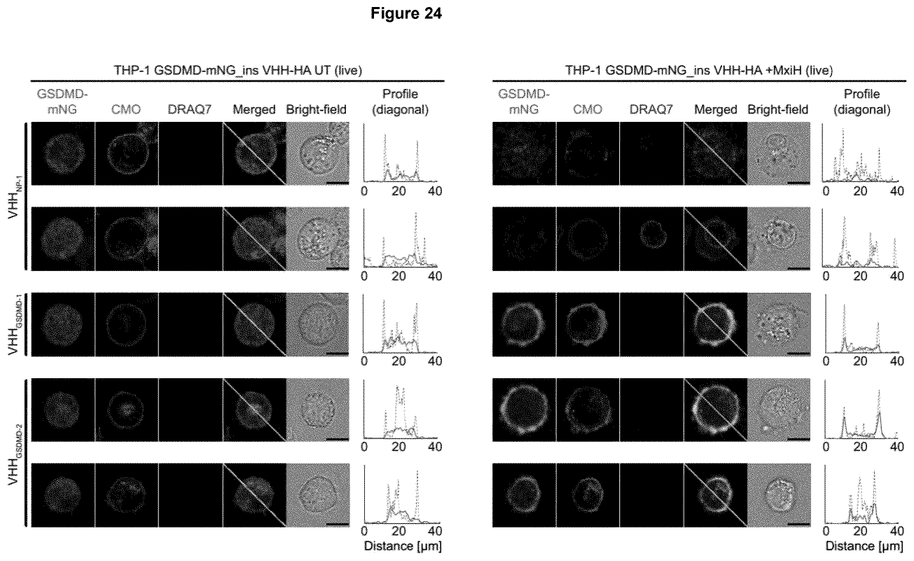

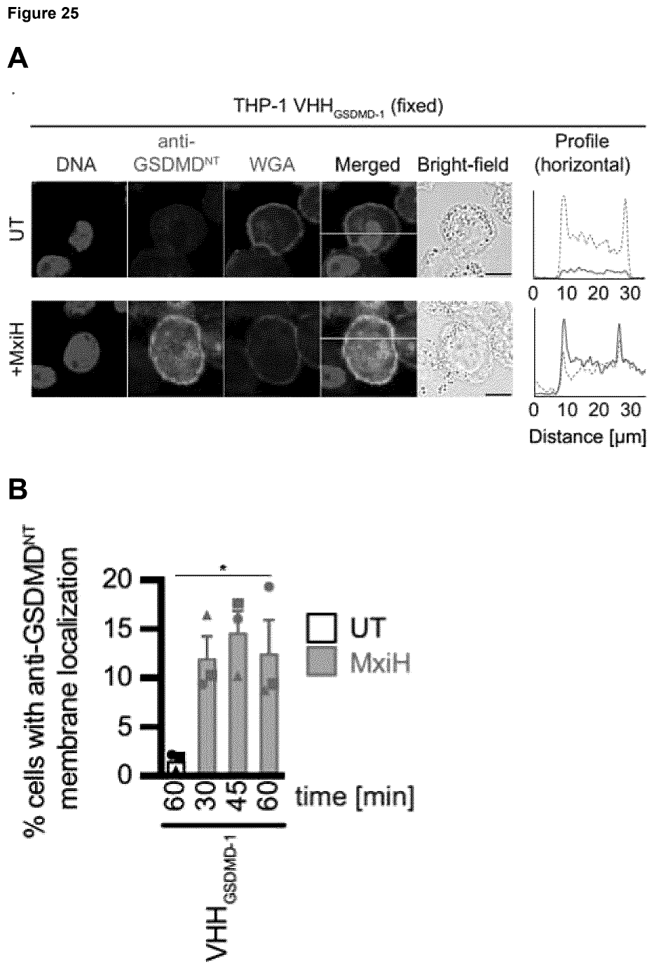

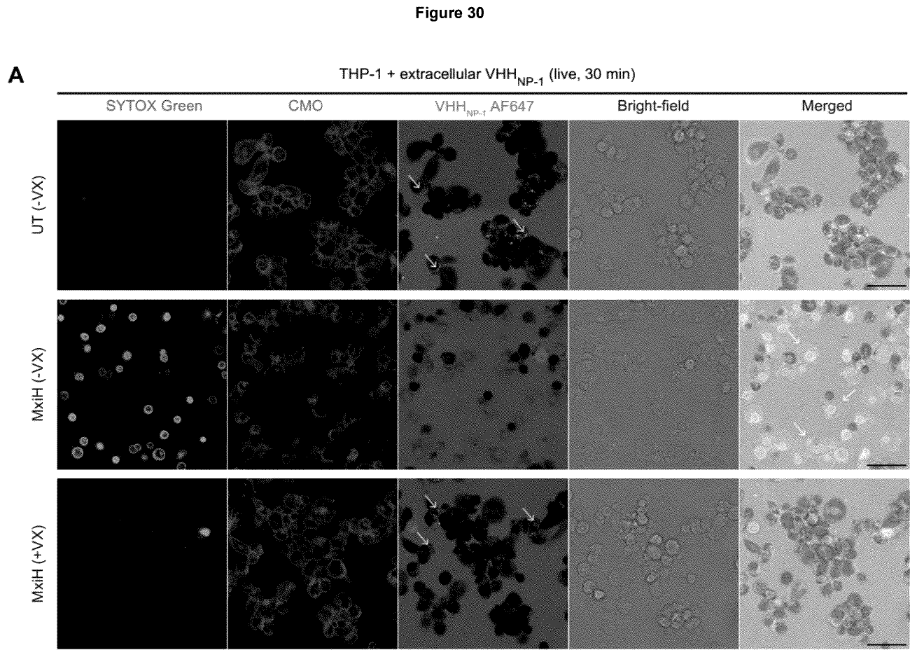

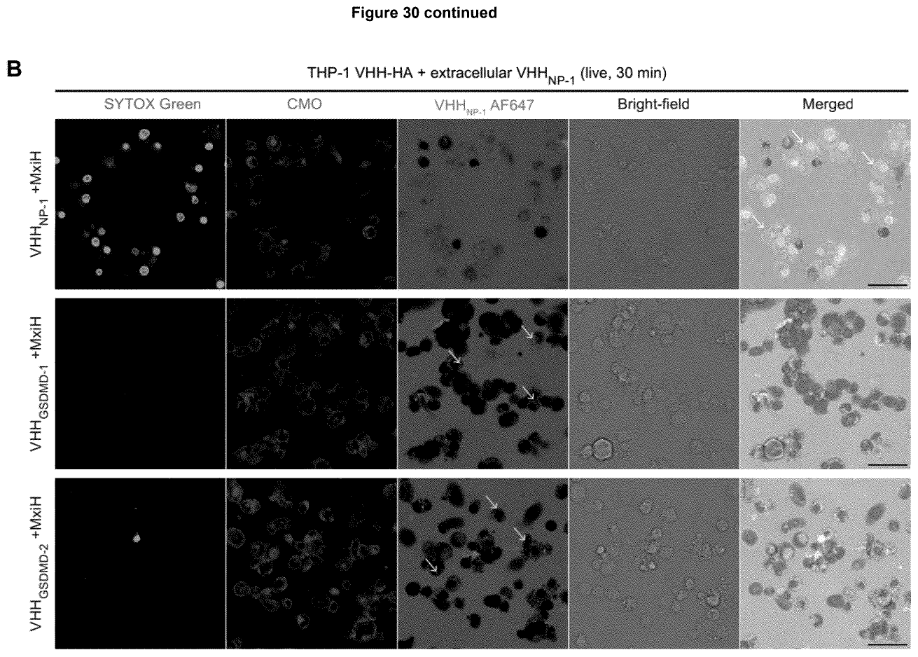

Further data as provided in FIGS. 20 to 32 further showed that human GSDMD nanobodies do not bind to murine GsdmD in LUMIER assays (FIG. 20A). It was further found that cytosolically expressed VHH_{GSDMD-1} and VHH_{GSDMD-2} inhibit pyroptosis triggered by overexpressed GSDMD^{NT}, but not by its overexpressed related GSDME^{NT} (FIG. 21A), It was also established that cytosolically expressed VHH_{GSDMD-1} inhibits pyroptosis triggered by overexpressed murine GsdnD^{NT} (FIG. 21B). It was also found that recombinant extracellular VHH_{GSDMD-1} does not inhibit pyroptosis triggered in murine macrophages (FIG. 21C), which suggests that inhibition of murine GsdmD is less potent. The further data also provided lessons on GSDMD pore formation. VHH_{GSDMD-1} stabilizes monomeric GSDMD^{NT}-mCherry in plasma membrane of HEK293T cells. The quantification of membrane localization is shown in FIGS. 22A and 22B. The analysis of GSDMD point mutants supports that membrane localization can be interpreted as membrane insertion. It was also confirmed that VHH_{GSDMD-1} and VHH_{GSDMD-2} stabilize GSDMD^{NT}-mNeonGreen in plasma membrane after cleavage by caspase-1 in THP-1 cells. The quantification of membrane localization is shown in FIGS. 23A/B and FIG. 24. It was also confirmed that VHH_{GSDMD-1} stabilizes endogenous GSDMD^{NT} in plasma membrane after cleavage by caspase-1 in THP-1 cells (staining of cleaved GSDMD with antibody and quantification of membrane localization is shown in FIGS. 25A/B).

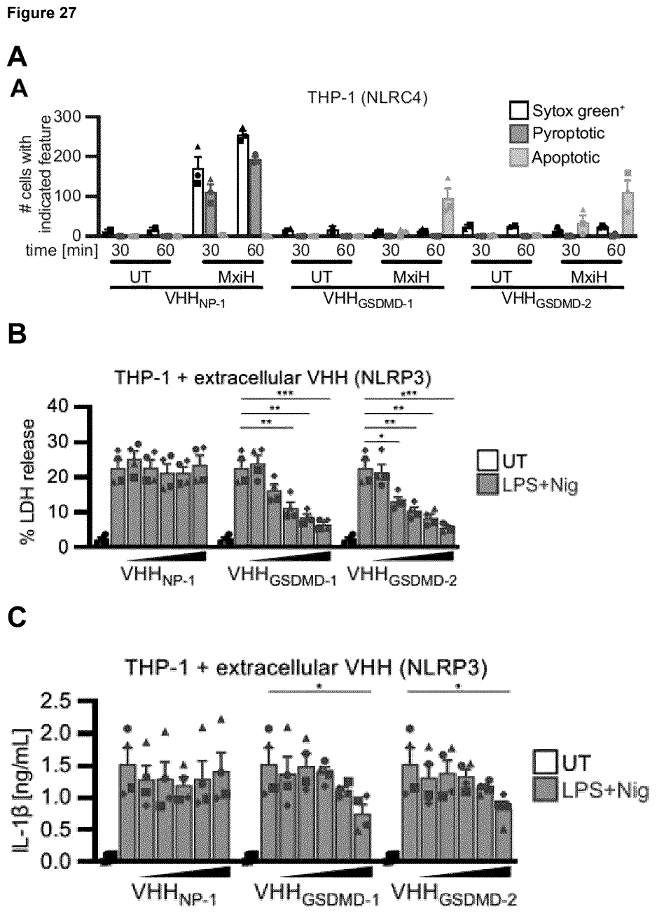

Additional data shows that GSDMD pores are rapidly removed from plasma membrane, likely by a mechanism that involves endocytosis of plasma membrane streches with GSDMD pores (FIG. 26A/B). It is also shown that cytosolically expressed VHH_{GSDMD-1} and VHH_{GSDMD-2} shift cells from pyroptosis to apoptosis, as quantified in FIG. 27A.

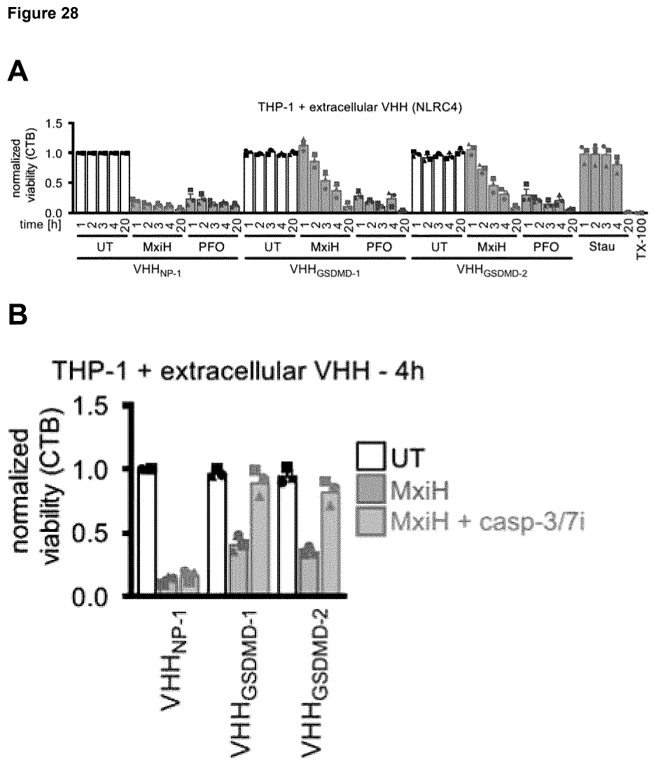

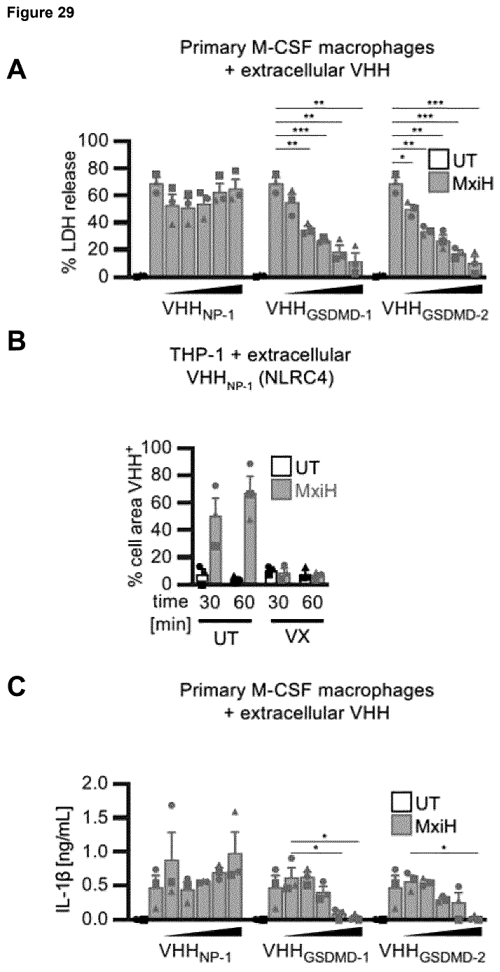

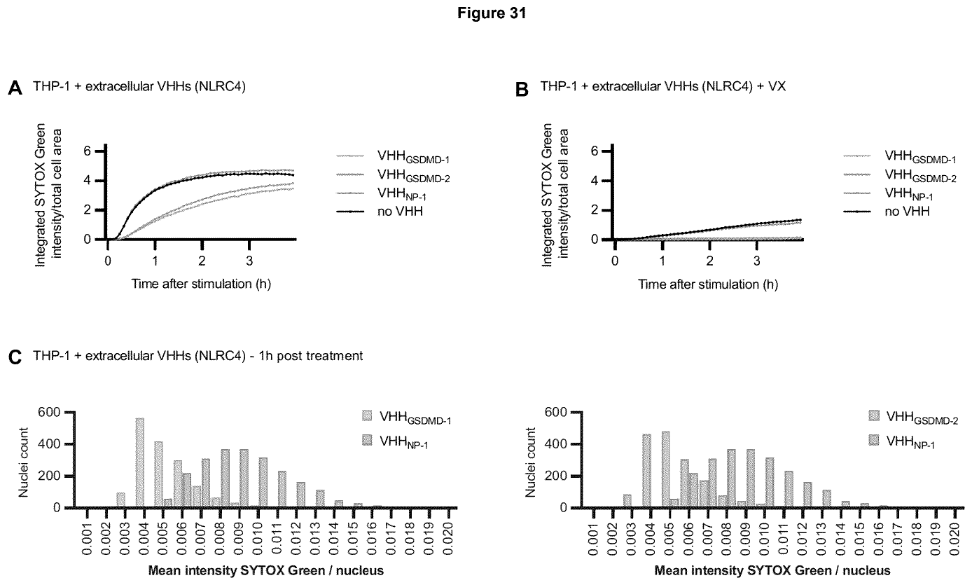

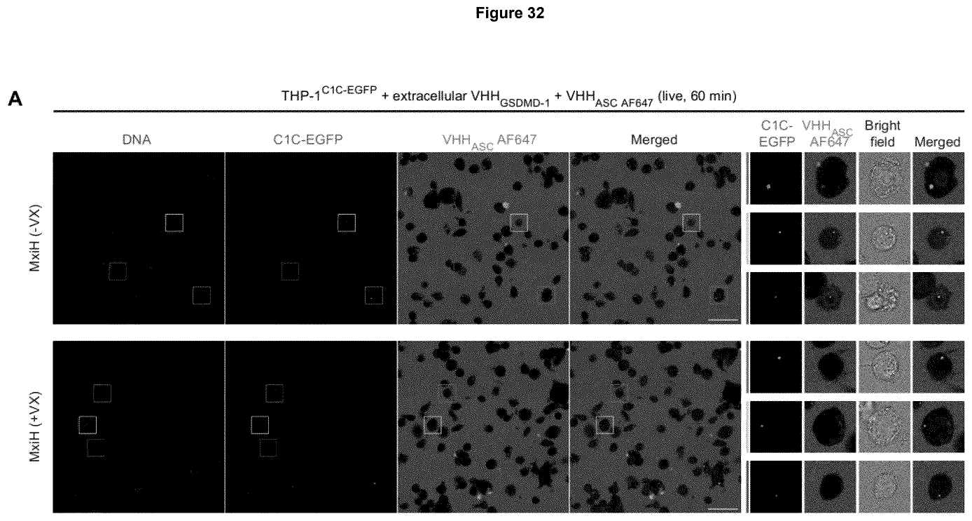

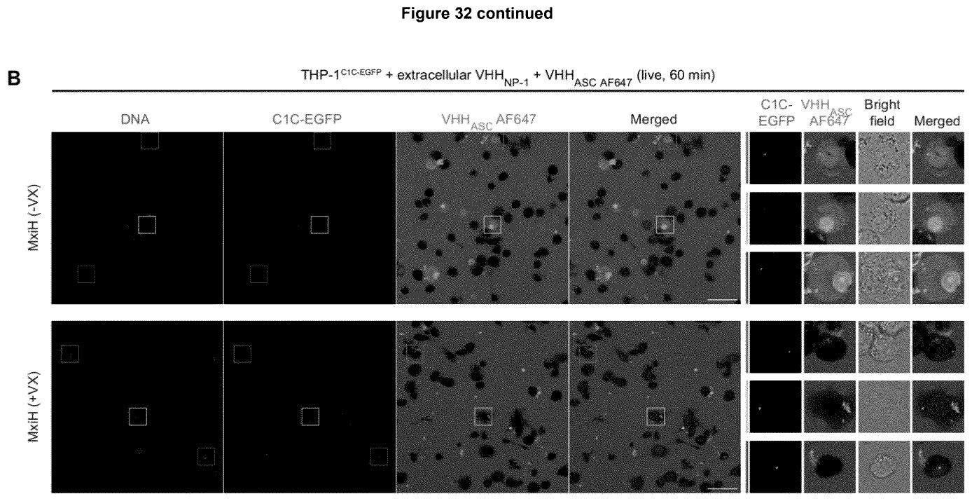

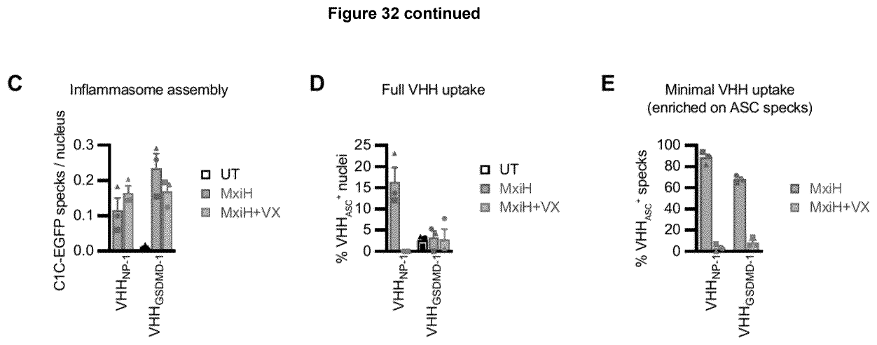

The additional data is also relevant for therapeutic applications. It is shown that extracellular VHH_{GSDMD-1} and VHH_{GSDMD-2} also inhibit cell death after activation of NLRP3 inflammasomes (FIG. 27 B/C). The inhibition/delay of cell death by extracellular VHH_{GSDMD-1} and VHH_{GSDMD-2} also confirmed using different readout independent of plasma membrane integrity (FIG. 28 A/B). The data also confirms the observed shift from pyroptosis to apoptosis. It was also confirmed that uptake of nanobodies into pyroptotic cells relies on GSDMD pores (FIG. 29 A/B, 30 A/B). In this regard, pyroptotic cells are cells with GSDMD pores that are round-up, but not ruptured. It was further proved and quantified that cells with sub-lytic GSDMD pores [i.e. cells stimulated in presence of extracellular GSDMD nanobodies] take up DNA dyes (FIG. 31) and nanobodies (FIG. 32).

1. Single-domain antibody directed against gasdermin D (GSDMD), wherein the single-domain antibody comprises an amino acid sequence comprising framework region 1 (FR1), complementarity-determining region 1 (CDR1), FR2, CDR2, FR3, CDR3, and FR4, and wherein the single-domain antibody comprises
(a) CDR1 as defined by SEQ ID NO:2, CDR2 as defined by SEQ ID NO:4, and CDR3 as defined by SEQ ID NO:6; or
(b) CDR1 as defined by SEQ ID NO:10, CDR2 as defined by SEQ ID NO:12, and CDR3 as defined by SEQ ID NO:14; or
(c) CDR1 as defined by SEQ ID NO:18, CDR2 as defined by SEQ ID NO:20, and CDR3 as defined by SEQ ID NO:22; or
(d) CDR1 as defined by SEQ ID NO:26, CDR2 as defined by SEQ ID NO:28, and CDR3 as defined by SEQ ID NO:30; or
(e) CDR1 as defined by SEQ ID NO:34, CDR2 as defined by SEQ ID NO:36, and CDR3 as defined by SEQ ID NO:38; or
(f) CDR1 as defined by SEQ ID NO:42, CDR2 as defined by SEQ ID NO:44, and CDR3 as defined by SEQ ID NO:46; or
(g) CDR1 as defined by SEQ ID NO:50, CDR2 as defined by SEQ ID NO:52, and CDR3 as defined by SEQ ID NO:54; or
(h) CDR1 as defined by SEQ ID NO:58, CDR2 as defined by SEQ ID NO:60, and CDR3 as defined by SEQ ID NO:62; or
(i) CDR1 as defined by SEQ ID NO:66, CDR2 as defined by SEQ ID NO:68, and CDR3 as defined by SEQ ID NO:70; or
(j) CDR1 as defined by SEQ ID NO:74, CDR2 as defined by SEQ ID NO:76, and CDR3 as defined by SEQ ID NO:78; or
(k) CDR1 as defined by SEQ ID NO:82, CDR2 as defined by SEQ ID NO:84, and CDR3 as defined by SEQ ID NO:86; or
(l) CDR1 as defined by SEQ ID NO:90, CDR2 as defined by SEQ ID NO:92, and CDR3 as defined by SEQ ID NO:94; or
(m) CDR1 as defined by SEQ ID NO:98, CDR2 as defined by SEQ ID NO:100, and CDR3 as defined by SEQ ID NO:102; or
(n) CDR1 as defined by SEQ ID NO:106, CDR2 as defined by SEQ ID NO:108, and CDR3 as defined by SEQ ID NO:110; or
(o) CDR1 as defined by SEQ ID NO:114, CDR2 as defined by SEQ ID NO:116, and CDR3 as defined by SEQ ID NO:118; or
(p) CDR1 as defined by SEQ ID NO:122, CDR2 as defined by SEQ ID NO:124, and CDR3 as defined by SEQ ID NO:126; or
(q) CDR1 as defined by SEQ ID NO:130, CDR2 as defined by SEQ ID NO:132, and CDR3 as defined by SEQ ID NO:134; or
(r) CDR1 as defined by SEQ ID NO:138, CDR2 as defined by SEQ ID NO:140, and CDR3 as defined by SEQ ID NO:142; or
(s) CDR1 as defined by SEQ ID NO:146, CDR2 as defined by SEQ ID NO:148, and CDR3 as defined by SEQ ID NO:150; or
(t) CDR1 as defined by SEQ ID NO:154, CDR2 as defined by SEQ ID NO:156, and CDR3 as defined by SEQ ID NO:158; or
(u) CDR1 as defined by SEQ ID NO:162, CDR2 as defined by SEQ ID NO:164, and CDR3 as defined by SEQ ID NO:166; or
(v) CDR1 as defined by SEQ ID NO:170, CDR2 as defined by SEQ ID NO:172, and CDR3 as defined by SEQ ID NO:174; or
(w) CDR1 as defined by SEQ ID NO:178, CDR2 as defined by SEQ ID NO:180, and CDR3 as defined by SEQ ID NO:182.

2. The single-domain antibody directed against GSDMD of claim 1, wherein the single-domain antibody comprises an amino acid sequence selected from the group comprising amino acid sequences as defined in SEQ ID NO: 8, 16, 24, 32, 40, 48, 56, 64, 72, 80, 88, 96, 104, 112, 120, 128, 136, 144, 152, 160, 168, 176, or 184, or variants thereof, wherein the variant comprises an amino acid sequence, which is at least 80%, 90%, 95%, or 99% identical to an amino acid sequence selected from the group comprising amino acid sequences as defined in SEQ ID NO: 8, 16, 24, 32, 40, 48, 56, 64, 72, 80, 88, 96, 104, 112, 120, 128, 136, 144, 152, 160, 168, 176, or 184.

3. The single-domain antibody directed against GSDMD of claim 1 or 2, wherein the single-domain antibody is capable of specifically binding an epitope within the N-terminal domain of GSDMD, wherein the epitope is a discontinuous epitope defined by amino acids L16, H18, G19, E21, F22, Q75, G77, R78, F80, S122, S124, D126, P127, N128, Q131, E162, R217, L231, and F232 of sequence of GSDMD (SEQ ID NO:185):
MGSAFERVVRRVVQELDHGGEFIPVTSLQSSTGFQPYCLVVRKPSSSWF
WKPRYKCVNLSIKDILEPDAAEPDVQRGRSFHFYDAMDGQIQGSVELAA
PGQAKIAGGAAVSDSSSTSMNVYSLSVDPNTWQTLLHERHLRQPEHKVL
QQLRSRGDNVYVVTEVLQTQKEVEVTRTHKREGSGRFSLPGATCLQGEG
QGHLSQKKTVTIPSGSTLAFRVAQLVIDSDLDVLLFPDKKQRTFQPPAT
GHKRSTSEGAWPQLPSGLSMMRCLHNFLTDGVPAEGAFTEDFQGLRAEV
ETISKELELLDRELCQLLLEGLEGVLRDQLALRALEEALEQGQSLGPVE
PLDGPAGAVLECLVLSSGMLVPELAIPVVYLLGALTMLSETQHKLLAEA
LESQTLLGPLELVGSLLEQSAPWQERSTMSLPPGLLGNSWGEGAPAWVL
LDECGLELGEDTPHVCWEPQAQGRMCALYASLALLSGLSQEPH.

4. Polynucleotide encoding the single-domain antibody of any one of claims 1 to 3.

5. The polynucleotide acid of claim 4, wherein the polynucleotide is selected from RNA, such as mRNA, DNA, such as genomic DNA, cDNA, or synthetic DNA, analogs thereof, or a combination thereof, wherein preferably the polynucleotide is mRNA.

6. The single-domain antibody directed against GSDMD of claim 1 to 3, wherein the single-domain antibody is capable of binding to cytosolic GSDMD in a cell, and thereby inhibiting pyroptosis of the cell, wherein the single-domain antibody directed against GSDMD is produced by the cell upon transfecting the cell with the polynucleotide of claim 4 or 5.

7. Host cell comprising the polynucleotide of claim 4 or 5.

8. Pharmaceutical composition comprising the single-domain antibody directed against GSDMD of any one of claims 1 to 3 or the polynucleotide of claim 4 or 5, and a pharmaceutically acceptable carrier.

9. The single-domain antibody directed against GSDMD of any one of claims 1 to 3 or the polynucleotide of claim 4 or 5, or the pharmaceutical composition of claim 8 for use in therapy.

10. The single-domain antibody directed against GSDMD of any one of claims 1 to 3 or the polynucleotide of claim 4 or 5 or the pharmaceutical composition of claim 8 for use in a method of treating or preventing an inflammatory disease or condition in a subject, wherein the inflammatory disease or condition is selected from the group comprising an acute inflammation, a chronic inflammation, sepsis, loss of the blood-brain barrier, in particular caused by sepsis, septic shock, non-alcoholic steatohepatitis, lung cancer, Familial Mediterranean Fever (FMF), autoinflammatory diseases, Cryoprin associated periodic syndrome (CAPS), non-alcoholic fatty liver disease, Alzheimer's disease, Parkinson's disease, age related macular degeneration, atherosclerosis, asthma and allergy airway inflammation, gout, Crohn's disease, ulcerative colitis, inflammatory bowel disease, hypertension, nephropathy, myocardial infarction, multiple sclerosis, experimental autoimmune encephalitis, hyperinflammation following influenza infection, graft versus host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury.

11. Method for determining the presence or absence of GSDMD oligomers in a sample obtained from a subject, wherein the method comprises contacting the sample with a reporter system comprising two single-domain antibodies directed against GSDMD, wherein both single-domain antibodies compete for the same epitope in GSDMD, wherein binding of the single-domain antibodies to the epitope does not affect the capability of GSDMD to oligomerize, and wherein upon binding of both single-domain antibodies to GSDMD, the reporter system provides a reporter signal that can be detected indicating the presence of GSDMD oligomers.

12. Method of claim 11, wherein the sample is selected from the group comprising serum, plasma, and whole blood.

13. Method of claim 11 or 12, wherein the presence or absence is determined with a sandwich ELISA, wherein the first single-domain antibody directed against GSDMD is used as primary antibody to capture GSDMD, and wherein the second single-domain antibody directed against GSDMD is used as a secondary antibody, wherein the secondary antibody is labelled, or with an HTRF/FRET based assay, wherein a first fluorescently labelled single-domain antibody directed against GSDMD is used as fluorescence donor, and a second fluorescently labelled single-domain antibody directed against GSDMD is used as fluorescence acceptor.

14. Method of any one of claims 11 to 13, wherein the first single-domain antibody directed against GSDMD and the second single-domain antibody directed against GSDMD is the single-domain antibody of any one of claims 1 to 3, wherein the first single-domain antibody and the second single-domain antibody can be the same single-domain antibody or different single-domain antibodies.

15. Single-domain antibody directed against gasdermin D (GSDMD) or polynucleotide encoding the single-domain antibody or pharmaceutical composition comprising the single-domain antibody or the polynucleotide for use in a method of treating or preventing an inflammatory disease or condition in a subject, wherein the inflammatory disease or condition is selected from the group comprising an acute inflammation, a chronic inflammation, sepsis, loss of the blood-brain barrier, in particular caused by sepsis, septic shock, non-alcoholic steatohepatitis, lung cancer, Familial Mediterranean Fever (FMF), autoinflammatory diseases, Cryoprin associated periodic syndrome (CAPS), non-alcoholic fatty liver disease, Alzheimer's disease, Parkinson's disease, age related macular degeneration, atherosclerosis, asthma and allergy airway inflammation, gout, Crohn's disease, ulcerative colitis, inflammatory bowel disease, hypertension, nephropathy, myocardial infarction, multiple sclerosis, experimental autoimmune encephalitis, hyperinflammation following influenza infection, graft versus host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury, wherein a presence of GSDMD oligomers has been determined in a sample obtained from the subject.

16. Single-domain antibody directed against gasdermin D (GSDMD) or polynucleotide encoding the single-domain antibody for use of claim 15, wherein the single-domain antibody directed against GSDMD is the single-domain antibody of any one of claims 1 to 3.
